SEQUENCE LISTING (187 sequences)

SEQ ID NO: 1 - protein, 15 aa, Artificial sequence
  Synthetic polypeptide
MSTNPKPQRKTKRNT

SEQ ID NO: 2 - protein, 15 aa, Artificial sequence
  Synthetic polypeptide
PKPQRKTKRNTNRRP

SEQ ID NO: 3 - protein, 15 aa, Artificial sequence
  Synthetic polypeptide
RKTKRNTNRRPQDVK

SEQ ID NO: 4 - protein, 15 aa, Artificial sequence
  Synthetic polypeptide
RNTNRRPQDVKFPGG

SEQ ID NO: 5 - protein, 15 aa, Artificial sequence
  Synthetic polypeptide
RRPQDVKFPGGGQIV

SEQ ID NO: 6 - protein, 15 aa, Artificial sequence
  Synthetic polypeptide
DVKFPGGGQIVGGVY

SEQ ID NO: 7 - protein, 15 aa, Artificial sequence
  Synthetic polypeptide
PGGGQIVGGVYLLPR

SEQ ID NO: 8 - protein, 15 aa, Artificial sequence
  Synthetic polypeptide
QIVGGVYLLPRRGPR

SEQ ID NO: 9 - protein, 15 aa, Artificial sequence
  Synthetic polypeptide
GVYLLPRRGPRLGVR

SEQ ID NO: 10 - protein, 15 aa, Artificial sequence
  Synthetic polypeptide
LPRRGPRLGVRATRK

SEQ ID NO: 11 - protein, 15 aa, Artificial sequence
  Synthetic polypeptide
GPRLGVRATRKTSER

SEQ ID NO: 12 - protein, 15 aa, Artificial sequence
  Synthetic polypeptide
GVRATRKTSERSQPR

SEQ ID NO: 13 - protein, 15 aa, Artificial sequence
  Synthetic polypeptide
TRKTSERSQPRGRRQ

SEQ ID NO: 14 - protein, 15 aa, Artificial sequence
  Synthetic polypeptide
SERSQPRGRRQPIPK

SEQ ID NO: 15 - protein, 15 aa, Artificial sequence
  Synthetic polypeptide
QPRGRRQPIPKARRP

SEQ ID NO: 16 - protein, 15 aa, Artificial sequence
  Synthetic polypeptide
RRQPIPKARRPEGRT

SEQ ID NO: 17 - protein, 15 aa, Artificial sequence
  Synthetic polypeptide
IPKARRPEGRTWAQP

SEQ ID NO: 18 - protein, 15 aa, Artificial sequence
  Synthetic polypeptide
RRPEGRTWAQPGYPW

SEQ ID NO: 19 - protein, 15 aa, Artificial sequence
  Synthetic polypeptide
GRTWAQPGYPWPLYG

SEQ ID NO: 20 - protein, 15 aa, Artificial sequence
  Synthetic polypeptide
AQPGYPWPLYGNEGC

SEQ ID NO: 21 - protein, 15 aa, Artificial sequence
  Synthetic polypeptide
YPWPLYGNEGCGWAG

SEQ ID NO: 22 - protein, 15 aa, Artificial sequence
  Synthetic polypeptide
LYGNEGCGWAGWLLS

SEQ ID NO: 23 - protein, 15 aa, Artificial sequence
  Synthetic polypeptide
EGCGWAGWLLSPRGS

SEQ ID NO: 24 - protein, 15 aa, Artificial sequence
  Synthetic polypeptide
WAGWLLSPRGSRPSW

SEQ ID NO: 25 - protein, 15 aa, Artificial sequence
  Synthetic polypeptide
LLSPRGSRPSWGPTD

SEQ ID NO: 26 - protein, 15 aa, Artificial sequence
  Synthetic polypeptide
RGSRPSWGPTDPRRR

SEQ ID NO: 27 - protein, 15 aa, Artificial sequence
  Synthetic polypeptide
PSWGPTDPRRRSRNL

SEQ ID NO: 28 - protein, 15 aa, Artificial sequence
  Synthetic polypeptide
PTDPRRRSRNLGKVI

SEQ ID NO: 29 - protein, 15 aa, Artificial sequence
  Synthetic polypeptide
RRRSRNLGKVIDTLT

SEQ ID NO: 30 - protein, 15 aa, Artificial sequence
  Synthetic polypeptide
RNLGKVIDTLTCGFA

SEQ ID NO: 31 - protein, 15 aa, Artificial sequence
  Synthetic polypeptide
KVIDTLTCGFADLMG

SEQ ID NO: 32 - protein, 15 aa, Artificial sequence
  Synthetic polypeptide
TLTCGFADLMGYIPL

SEQ ID NO: 33 - protein, 15 aa, Artificial sequence
  Synthetic polypeptide
GFADLMGYIPLVGAP

SEQ ID NO: 34 - protein, 15 aa, Artificial sequence
  Synthetic polypeptide
LMGYIPLVGAPLGGA

SEQ ID NO: 35 - protein, 15 aa, Artificial sequence
  Synthetic polypeptide
IPLVGAPLGGAARAL

SEQ ID NO: 36 - protein, 15 aa, Artificial sequence
  Synthetic polypeptide
GAPLGGAARALAHGV

SEQ ID NO: 37 - protein, 15 aa, Artificial sequence
  Synthetic polypeptide
GGAARALAHGVRVLE

SEQ ID NO: 38 - protein, 15 aa, Artificial sequence
  Synthetic polypeptide
RALAHGVRVLEDGVN

SEQ ID NO: 39 - protein, 15 aa, Artificial sequence
  Synthetic polypeptide
HGVRVLEDGVNYATG

SEQ ID NO: 40 - protein, 15 aa, Artificial sequence
  Synthetic polypeptide
VLEDGVNYATGNLPG

SEQ ID NO: 41 - protein, 15 aa, Artificial sequence
  Synthetic polypeptide
GVNYATGNLPGCSFS

SEQ ID NO: 42 - protein, 15 aa, Artificial sequence
  Synthetic polypeptide
ATGNLPGCSFSIFLL

SEQ ID NO: 43 - protein, 15 aa, Artificial sequence
  Synthetic polypeptide
LPGCSFSIFLLALLS

SEQ ID NO: 44 - protein, 15 aa, Artificial sequence
  Synthetic polypeptide
SFSIFLLALLSCLTV

SEQ ID NO: 45 - protein, 15 aa, Artificial sequence
  Synthetic polypeptide
FLLALLSCLTVPASA

SEQ ID NO: 46 - protein, 15 aa, Artificial sequence
  Synthetic polypeptide
MSTNPKPQRKPNVTP

SEQ ID NO: 47 - protein, 15 aa, Artificial sequence
  Synthetic polypeptide
PNVTPTVAHRTSSSR

SEQ ID NO: 48 - protein, 15 aa, Artificial sequence
  Synthetic polypeptide
TSSSRVAVRSLVEFT

SEQ ID NO: 49 - protein, 15 aa, Artificial sequence
  Synthetic polypeptide
LVEFTCCRAGALDWV

SEQ ID NO: 50 - protein, 15 aa, Artificial sequence
  Synthetic polypeptide
ALDWVCARRGRLPSG

SEQ ID NO: 51 - protein, 15 aa, Artificial sequence
  Synthetic polypeptide
RLPSGRNLEVDVSLS

SEQ ID NO: 52 - protein, 15 aa, Artificial sequence
  Synthetic polypeptide
DVSLSPRHVGPRAGP

SEQ ID NO: 53 - protein, 15 aa, Artificial sequence
  Synthetic polypeptide
PRAGPGLSPGTLGPS

SEQ ID NO: 54 - protein, 15 aa, Artificial sequence
  Synthetic polypeptide
TLGPSMAMRVAGGRD

SEQ ID NO: 55 - protein, 15 aa, Artificial sequence
  Synthetic polypeptide
AGGRDGSCLPVALGL

SEQ ID NO: 56 - protein, 15 aa, Artificial sequence
  Synthetic polypeptide
VALGLAGAPQTPGVG

SEQ ID NO: 57 - protein, 15 aa, Artificial sequence
  Synthetic polypeptide
TPGVGRAIWVRSSIP

SEQ ID NO: 58 - protein, 15 aa, Artificial sequence
  Synthetic polypeptide
RSSIPLRAASPTSWG

SEQ ID NO: 59 - protein, 15 aa, Artificial sequence
  Synthetic polypeptide
PTSWGTYRSSAPLLE

SEQ ID NO: 60 - protein, 15 aa, Artificial sequence
  Synthetic polypeptide
APLLEALPGPWRMAS

SEQ ID NO: 61 - protein, 15 aa, Artificial sequence
  Synthetic polypeptide
LPGPWRMASGFWKTA

SEQ ID NO: 62 - protein, 15 aa, Artificial sequence
  Synthetic polypeptide
LSTTGEIPFYGKAIP

SEQ ID NO: 63 - protein, 14 aa, Artificial sequence
  Synthetic polypeptide
GINAVAYYRGLDVS

SEQ ID NO: 64 - protein, 15 aa, Artificial sequence
  Synthetic polypeptide
VAYYRGLDVSVIPTS

SEQ ID NO: 65 - protein, 15 aa, Artificial sequence
  Synthetic polypeptide
EIPFYGKAIPLEVIK

SEQ ID NO: 66 - protein, 15 aa, Artificial sequence
  Synthetic polypeptide
SVIDCNTCVTQTVDF

SEQ ID NO: 67 - protein, 15 aa, Artificial sequence
  Synthetic polypeptide
SSDLYLVTRHADVIP

SEQ ID NO: 68 - protein, 15 aa, Artificial sequence
  Synthetic polypeptide
PTPLLYRLGAVQNEV

SEQ ID NO: 69 - protein, 15 aa, Artificial sequence
  Synthetic polypeptide
RRGRTGRGKPGIYRF

SEQ ID NO: 70 - protein, 15 aa, Artificial sequence
  Synthetic polypeptide
RGVAKAVDFIPVENL

SEQ ID NO: 71 - protein, 15 aa, Artificial sequence
  Synthetic polypeptide
MWKCLIRLKPTLHGP

SEQ ID NO: 72 - protein, 15 aa, Artificial sequence
  Synthetic polypeptide
QTFLATCINGVCWTV

SEQ ID NO: 73 - protein, 20 aa, Artificial sequence
  Synthetic polypeptide
STWVLVGGVLAALAAYCLST

SEQ ID NO: 74 - protein, 20 aa, Artificial sequence
  Synthetic polypeptide
YCLSTGCVVIVGRIVLSGKP

SEQ ID NO: 75 - protein, 20 aa, Artificial sequence
  Synthetic polypeptide
LSGKPAIIPDREVLYQEFDE

SEQ ID NO: 76 - protein, 19 aa, Artificial sequence
  Synthetic polypeptide
QEFDEMEECSQHLPYIEQG

SEQ ID NO: 77 - protein, 20 aa, Artificial sequence
  Synthetic polypeptide
SQHLPYIEQGMMLAEQFKQK

SEQ ID NO: 78 - protein, 20 aa, Artificial sequence
  Synthetic polypeptide
QFKQKALGLLQTASRHAEVI

SEQ ID NO: 79 - protein, 20 aa, Artificial sequence
  Synthetic polypeptide
HAEVITPAVQTNWQKLEVFW

SEQ ID NO: 80 - protein, 20 aa, Artificial sequence
  Synthetic polypeptide
LEVFWAKHMWNFISGIQYLA

SEQ ID NO: 81 - protein, 20 aa, Artificial sequence
  Synthetic polypeptide
IQYLAGLSTLPGNPAIASLM

SEQ ID NO: 82 - protein, 20 aa, Artificial sequence
  Synthetic polypeptide
IASLMAFTAAVTSPLTTGQT

SEQ ID NO: 83 - protein, 20 aa, Artificial sequence
  Synthetic polypeptide
TTGQTLLFNILGGWVAAQLA

SEQ ID NO: 84 - protein, 20 aa, Artificial sequence
  Synthetic polypeptide
AAQLAAPGAATAFVGAGLAG

SEQ ID NO: 85 - protein, 20 aa, Artificial sequence
  Synthetic polypeptide
AGLAGAAIGSVGLGKVLVDI

SEQ ID NO: 86 - protein, 20 aa, Artificial sequence
  Synthetic polypeptide
VLVDILAGYGAGVAGALVAF

SEQ ID NO: 87 - protein, 20 aa, Artificial sequence
  Synthetic polypeptide
ALVAFKIMSGEVPSTEDLVN

SEQ ID NO: 88 - protein, 20 aa, Artificial sequence
  Synthetic polypeptide
EDLVNLLPAILSPGALVVGV

SEQ ID NO: 89 - protein, 20 aa, Artificial sequence
  Synthetic polypeptide
LVVGVVCAAILRRHVGPGEG

SEQ ID NO: 90 - protein, 20 aa, Artificial sequence
  Synthetic polypeptide
GPGEGAVQWMNRLIAFASRG

SEQ ID NO: 91 - protein, 20 aa, Artificial sequence
  Synthetic polypeptide
FASRGNHVSPTHYVPESDAA

SEQ ID NO: 92 - protein, 20 aa, Artificial sequence
  Synthetic polypeptide
ESDAAARVTAILSSLTVTQL

SEQ ID NO: 93 - protein, 20 aa, Artificial sequence
  Synthetic polypeptide
CSGSWLRDIWDWICEVLSDF

SEQ ID NO: 94 - protein, 20 aa, Artificial sequence
  Synthetic polypeptide
VLSDFKTWLKAKLMPQLPGI

SEQ ID NO: 95 - protein, 20 aa, Artificial sequence
  Synthetic polypeptide
QLPGIPFVSCQRGYRGVWRG

SEQ ID NO: 96 - protein, 20 aa, Artificial sequence
  Synthetic polypeptide
GVWRGDGIMHTRCHCGAEIT

SEQ ID NO: 97 - protein, 20 aa, Artificial sequence
  Synthetic polypeptide
GAEITGHVKNGTMRIVGPRT

SEQ ID NO: 98 - protein, 20 aa, Artificial sequence
  Synthetic polypeptide
VGPRTCRNMWSGTFPINAYT

SEQ ID NO: 99 - protein, 20 aa, Artificial sequence
  Synthetic polypeptide
INAYTTGPCTPLPAPNYKFA

SEQ ID NO: 100 - protein, 20 aa, Artificial sequence
  Synthetic polypeptide
NYKFALWRVSAEEYVEIRRV

SEQ ID NO: 101 - protein, 20 aa, Artificial sequence
  Synthetic polypeptide
EIRRVGDFHYVSGMTTDNLK

SEQ ID NO: 102 - protein, 20 aa, Artificial sequence
  Synthetic polypeptide
TDNLKCPCQIPSPEFFTELD

SEQ ID NO: 103 - protein, 20 aa, Artificial sequence
  Synthetic polypeptide
FTELDGVRLHRFAPPCKPLL

SEQ ID NO: 104 - protein, 20 aa, Artificial sequence
  Synthetic polypeptide
CKPLLREEVSFRVGLHEYPV

SEQ ID NO: 105 - protein, 20 aa, Artificial sequence
  Synthetic polypeptide
HEYPVGSQLPCEPEPDVAVL

SEQ ID NO: 106 - protein, 20 aa, Artificial sequence
  Synthetic polypeptide
DVAVLTSMLTDPSHITAEAA

SEQ ID NO: 107 - protein, 20 aa, Artificial sequence
  Synthetic polypeptide
TAEAAGRRLARGSPPSMASS

SEQ ID NO: 108 - protein, 20 aa, Artificial sequence
  Synthetic polypeptide
SMASSSASQLSAPSLKATCT

SEQ ID NO: 109 - protein, 20 aa, Artificial sequence
  Synthetic polypeptide
KATCTANHDSPDAELIEANL

SEQ ID NO: 110 - protein, 20 aa, Artificial sequence
  Synthetic polypeptide
IEANLLWRQEMGGNITRVES

SEQ ID NO: 111 - protein, 20 aa, Artificial sequence
  Synthetic polypeptide
TRVESENKVVILDSFDPLVA

SEQ ID NO: 112 - protein, 20 aa, Artificial sequence
  Synthetic polypeptide
DPLVAEEDEREVSVPAEILR

SEQ ID NO: 113 - protein, 20 aa, Artificial sequence
  Synthetic polypeptide
AEILRKSRRFARALPVWARP

SEQ ID NO: 114 - protein, 20 aa, Artificial sequence
  Synthetic polypeptide
VWARPDYNPPLVETWKKPDY

SEQ ID NO: 115 - protein, 20 aa, Artificial sequence
  Synthetic polypeptide
KKPDYEPPVVHGCPLPPPRS

SEQ ID NO: 116 - protein, 20 aa, Artificial sequence
  Synthetic polypeptide
PPPRSPPVPPPRKKRTVVLT

SEQ ID NO: 117 - protein, 20 aa, Artificial sequence
  Synthetic polypeptide
TVVLTESTLSTALAELATKS

SEQ ID NO: 118 - protein, 20 aa, Artificial sequence
  Synthetic polypeptide
LATKSFGSSSTSGITGDNTT

SEQ ID NO: 119 - protein, 20 aa, Artificial sequence
  Synthetic polypeptide
GDNTTTSSEPAPSGCPPDSD

SEQ ID NO: 120 - protein, 20 aa, Artificial sequence
  Synthetic polypeptide
PPDSDVESYSSMPPLEGEPG

SEQ ID NO: 121 - protein, 29 aa, Artificial sequence
  Synthetic polypeptide
EGEPGDPDLSDGSWSTVSSGADTEDVVCC

SEQ ID NO: 122 - protein, 20 aa, Artificial sequence
  Synthetic polypeptide
SMSYSWTGALVTPCAAEEQK

SEQ ID NO: 123 - protein, 20 aa, Artificial sequence
  Synthetic polypeptide
AEEQKLPINALSNSLLRHHN

SEQ ID NO: 124 - protein, 20 aa, Artificial sequence
  Synthetic polypeptide
LRHHNLVYSTTSRSACQRQK

SEQ ID NO: 125 - protein, 20 aa, Artificial sequence
  Synthetic polypeptide
CQRQKKVTFDRLQVLDSHYQ

SEQ ID NO: 126 - protein, 20 aa, Artificial sequence
  Synthetic polypeptide
DSHYQDVLKEVKAAASKVKA

SEQ ID NO: 127 - protein, 20 aa, Artificial sequence
  Synthetic polypeptide
SKVKANLLSVEEACSLTPPH

SEQ ID NO: 128 - protein, 20 aa, Artificial sequence
  Synthetic polypeptide
LTPPHSAKSKFGYGAKDVRC

SEQ ID NO: 129 - protein, 20 aa, Artificial sequence
  Synthetic polypeptide
KDVRCHARKAVAHINSVWKD

SEQ ID NO: 130 - protein, 20 aa, Artificial sequence
  Synthetic polypeptide
SVWKDLLEDSVTPIDTTIMA

SEQ ID NO: 131 - protein, 20 aa, Artificial sequence
  Synthetic polypeptide
TTIMAKNEVFCVQPEKGGRK

SEQ ID NO: 132 - protein, 20 aa, Artificial sequence
  Synthetic polypeptide
KGGRKPARLIVFPDLGVRVC

SEQ ID NO: 133 - protein, 20 aa, Artificial sequence
  Synthetic polypeptide
GVRVCEKMALYDVVSKLPLA

SEQ ID NO: 134 - protein, 20 aa, Artificial sequence
  Synthetic polypeptide
KLPLAVMGSSYGFQYSPGQR

SEQ ID NO: 135 - protein, 20 aa, Artificial sequence
  Synthetic polypeptide
SPGQRVEFLVQAWKSKKTPM

SEQ ID NO: 136 - protein, 20 aa, Artificial sequence
  Synthetic polypeptide
KKTPMGFSYDTRCFDSTVTE

SEQ ID NO: 137 - protein, 20 aa, Artificial sequence
  Synthetic polypeptide
STVTESDIRTEEAIYQCCDL

SEQ ID NO: 138 - protein, 20 aa, Artificial sequence
  Synthetic polypeptide
QCCDLDPQARVAIKSLTERL

SEQ ID NO: 139 - protein, 20 aa, Artificial sequence
  Synthetic polypeptide
LTERLYVGGPLTNSRGENCG

SEQ ID NO: 140 - protein, 20 aa, Artificial sequence
  Synthetic polypeptide
GENCGYRRCRASGVLTTSCG

SEQ ID NO: 141 - protein, 20 aa, Artificial sequence
  Synthetic polypeptide
TTSCGNTLTCYIKARAACRA

SEQ ID NO: 142 - protein, 20 aa, Artificial sequence
  Synthetic polypeptide
AACRAAGLQDCTMLVCGDDL

SEQ ID NO: 143 - protein, 20 aa, Artificial sequence
  Synthetic polypeptide
CGDDLVVICESAGVQEDAAN

SEQ ID NO: 144 - protein, 20 aa, Artificial sequence
  Synthetic polypeptide
EDAANLRAFTEAMTRYSAPP

SEQ ID NO: 145 - protein, 20 aa, Artificial sequence
  Synthetic polypeptide
YSAPPGDPPQPEYDLELITS

SEQ ID NO: 146 - protein, 20 aa, Artificial sequence
  Synthetic polypeptide
ELITSCSSNVSVAHDGAGKR

SEQ ID NO: 147 - protein, 20 aa, Artificial sequence
  Synthetic polypeptide
GAGKRVYYLTRDPTTPLARA

SEQ ID NO: 148 - protein, 20 aa, Artificial sequence
  Synthetic polypeptide
PLARAAWETARHTPVNSWLG

SEQ ID NO: 149 - protein, 20 aa, Artificial sequence
  Synthetic polypeptide
NSWLGNIIMFAPTLWARMIL

SEQ ID NO: 150 - protein, 20 aa, Artificial sequence
  Synthetic polypeptide
ARMILMTHFFSVLIARDQLE

SEQ ID NO: 151 - protein, 20 aa, Artificial sequence
  Synthetic polypeptide
RDQLEQALNCEIYGACYSIE

SEQ ID NO: 152 - protein, 20 aa, Artificial sequence
  Synthetic polypeptide
CYSIEPLDLPPIIQRLHGLS

SEQ ID NO: 153 - protein, 20 aa, Artificial sequence
  Synthetic polypeptide
LHGLSAFSLHSYSPGEINRV

SEQ ID NO: 154 - protein, 20 aa, Artificial sequence
  Synthetic polypeptide
EINRVAACLRKLGVPPLRAW

SEQ ID NO: 155 - protein, 20 aa, Artificial sequence
  Synthetic polypeptide
PLRAWRHRARSVRARLLSRG

SEQ ID NO: 156 - protein, 20 aa, Artificial sequence
  Synthetic polypeptide
LLSRGGRAAICGKYLFNWAV

SEQ ID NO: 157 - protein, 20 aa, Artificial sequence
  Synthetic polypeptide
FNWAVRTKLKLTPITAAGRL

SEQ ID NO: 158 - protein, 20 aa, Artificial sequence
  Synthetic polypeptide
AAGRLDLSGWFTAGYSGGDI

SEQ ID NO: 159 - protein, 20 aa, Artificial sequence
  Synthetic polypeptide
SGGDIYHSVSHARPRWFWFC

SEQ ID NO: 160 - protein, 20 aa, Artificial sequence
  Synthetic polypeptide
WFWFCLLLLAAGVGIYLLPN

SEQ ID NO: 161 - protein, 529 aa, Human adenovirus 5
MASREEEQRETTPERGRGAARRPPTMEDVSSPSPSPPPPRAPPKKRMRRRIESEDEEDSSQDALVPRTPSPRPSTSAADLAIAPKKKKKRPSPKPERPPSPEVIVDSEEEREDVALQMVGFSNPPVLIKHGKGGKRTVRRLNEDDPVARGMRTQEEEEEPSEAESEITVMNPLSVPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 162 - protein, 1198 aa, Human adenovirus 5
MALAQAHRARRLHAEAPDSGDQPPRRRVRQQPTRAAPAPARARRRRAPAPSPGGSGAPPTSGGSPASPLLDASSKDTPAAHRPPRGTVVAPRGCGLLQAIDAATNQPLEIRYHLDLARALTRLCEVNLQELPPDLTPRELQTMDSSHLRDVVIKLRPPRADIWTLGSRGVVVRSTVTPLEQPDGQGQAAEVEDHQPNPPGEGLKFPLCFLVRGRQVNLVQDVQPVHRCQYCARFYKSQHECSARRRDFYFHHINSHSSNWWREIQFFPIGSHPRTERLFVTYDVETYTWMGAFGKQLVPFMLVMKFGGDEPLVTAARDLAANLGWDRWEQDPLTFYCITPEKMAIGRQFRTFRDHLQMLMARDLWSSFVASNPHLADWALSEHGLSSPEELTYEELKKLPSIKGIPRFLELYIVGHNINGFDEIVLAAQVINNRSEVPGPFRITRNFMPRAGKILFNDVTFALPNPRSKKRTDFLLWEQGGCDDTDFKYQYLKVMVRDTFALTHTSLRKAAQAYALPVEKGCCAYQAVNQFYMLGSYRSEADGFPIQEYWKDREEFVLNRELWKKKGQDKYDIIKETLDYCALDVQVTAELVNKLRDSYASFVRDAVGLTDASFNVFQRPTISSNSHAIFRQIVFRAEQPARSNLGPDLLAPSHELYDYVRASIRGGRCYPTYLGILREPLYVYDICGMYASALTHPMPWGPPLNPYERALAARAWQQALDLQGCKIDYFDARLLPGVFTVDADPPDETQLDPLPPFCSRKGGRLCWTNERLRGEVATSVDLVTLHNRGWRVHLVPDERTTVFPEWRCVAREYVQLNIAAKERADRDKNQTLRSIAKLLSNALYGSFATKLDNKKIVFSDQMDAATLKGITAGQVNIKSSSFLETDNLSAEVMPAFQREYSPQQLALADSDAEESEDERAPTPFYSPPSGTPGHVAYTYKPITFLDAEEGDMCLHTLERVDPLVDNDRYPSHLASFVLAWTRAFVSEWSEFLYEEDRGTPLEDRPLKSVYGDTDSLFVTERGHRLMETRGKKRIKKHGGNLVFDPERPELTWLVECETVCGACGADAYSPESVFLAPKLYALKSLHCPSCGASSKGKLRAKGHAAEGLDYDTMVKCYLADAQGEDRQRFSTSRTSLKRTLASAQPGAHPFTVTQTTLTRTLRPWKDMTLARLDEHRLLPYSESRPNPRNEEICWIEMP

SEQ ID NO: 163 - protein, 671 aa, Human adenovirus 5
MALSVNDCARLTGQSVPTMEHFLPLRNIWNRVRDFPRASTTAAGITWMSRYIYGYHRLMLEDLAPGAPATLRWPLYRQPPPHFLVGYQYLVRTCNDYVFDSRAYSRLRYTELSQPGHQTVNWSVMANCTYTINTGAYHRFVDMDDFQSTLTQVQQAILAERVVADLALLQPMRGFGVTRMGGRGRHLRPNSAAAAAIDARDAGQEEGEEEVPVERLMQDYYKDLRRCQNEAWGMADRLRIQQAGPKDMVLLSTIRRLKTAYFNYIISSTSARNNPDRRPLPPATVLSLPCDCDWLDAFLERFSDPVDADSLRSLGGGVPTQQLLRCIVSAVSLPHGSPPPTHNRDMTGGVFQLRPRENGRAVTETMRRRRGEMIERFVDRLPVRRRRRRVPPPPPPPEEEEGEALMEEEIEEEEEAPVAFEREVRDTVAELIRLLEEELTVSARNSQFFNFAVDFYEAMERLEALGDINESTLRRWVMYFFVAEHTATTLNYLFQRLRNYAVFARHVELNLAQVVMRARDAEGGVVYSRVWNEGGLNAFSQLMARISNDLAATVERAGRGDLQEEEIEQFMAEIAYQDNSGDVQEILRQAAVNDTEIDSVELSFRFKLTGPVVFTQRRQIQEINRRVVAFASNLRAQHQLLPARGADVPLPPLPAGPEPPLPPGARPRHRF

SEQ ID NO: 164 - protein, 294 aa, Human adenovirus 5
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 165 - protein, 128 aa, Human adenovirus 5
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 166 - protein, 130 aa, Human adenovirus 5
MFERKMVSFSVVVPELTCLYLHEHDYDVLSFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 167 - protein, 116 aa, Human adenovirus 5
MIRCLRLKVEGALEQIFTMAGLNIRDLLRDILRRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 168 - protein, 114 aa, Human adenovirus 5
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 169 - protein, 150 aa, Human adenovirus 5
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 170 - protein, 149 aa, Human adenovirus 5
MRADREELDLPPPIGGVAIDVVKVEVPATGRTLVLAFVKTCAVLAAVHGLYILHEVDLTTAHKEAEWEFEPLAWRVWLVVFYFGCLSLTVWLLEGSYGGSDHHAARAQSPDVRARRSELDDNIAQMGAVHGLELPRRQVRRELLQVYLA

SEQ ID NO: 171 - protein, 415 aa, Human adenovirus 5
MHPVLRQMRPPPQQRQEQEQRQTCRAPSPPPTASGGATSAVDAAADGDYEPPRRRARHYLDLEEGEGLARLGAPSPERHPRVQLKRDTREAYVPRQNLFRDREGEEPEEMRDRKFHAGRELRHGLNRERLLREEDFEPDARTGISPARAHVAAADLVTAYEQTVNQEINFQKSFNNHVRTLVAREEVAIGLMHLWDFVSALEQNPNSKPLMAQLFLIVQHSRDNEAFRDALLNIVEPEGRWLLDLINILQSIVVQERSLSLADKVAAINYSMLSLGKFYARKIYHTPYVPIDKEVKIEGFYMRMALKVLTLSDDLGVYRNERIHKAVSVSRRRELSDRELMHSLQRALAGTGSGDREAESYFDAGADLRWAPSRRALEAAGAGPGLAVAPARAGNVGGVEEYDEDDEYEPEDGEY

SEQ ID NO: 172 - protein, 585 aa, Human adenovirus 5
MMQDATDPAVRAALQSQPSGLNSTDDWRQVMDRIMSLTARNPDAFRQQPQANRLSAILEAVVPARANPTHEKVLAIVNALAENRAIRPDEAGLVYDALLQRVARYNSGNVQTNLDRLVGDVREAVAQRERAQQQGNLGSMVALNAFLSTQPANVPRGQEDYTNFVSALRLMVTETPQSEVYQSGPDYFFQTSRQGLQTVNLSQAFKNLQGLWGVRAPTGDRATVSSLLTPNSRLLLLLIAPFTDSGSVSRDTYLGHLLTLYREAIGQAHVDEHTFQEITSVSRALGQEDTGSLEATLNYLLTNRRQKIPSLHSLNSEEERILRYVQQSVSLNLMRDGVTPSVALDMTARNMEPGMYASNRPFINRLMDYLHRAAAVNPEYFTNAILNPHWLPPPGFYTGGFEVPEGNDGFLWDDIDDSVFSPQPQTLLELQQREQAEAALRKESFRRPSSLSDLGAAAPRSDASSPFPSLIGSLTSTRTTRPRLLGEEEYLNNSLLQPQREKNLPPAFPNNGIESLVDKMSRWKTYAQEHRDVPGPRPPTRRQRHDRQRGLVWEDDDSADDSSVLDLGGSGNPFAHLRPRLGRMF

SEQ ID NO: 173 - protein, 571 aa, Human adenovirus 5
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 174 - protein, 62 aa, Human adenovirus 5
MRAARRLAAGIVTVPPRSRRRAAAAAAAAISAMTQGRRGNVYWVRDSVSGLRVPVRTRPPRN

SEQ ID NO: 175 - protein, 368 aa, Human adenovirus 5
MSKRKIKEEMLQVIAPEIYGPPKKEEQDYKPRKLKRVKKKKKDDDDELDDEVELLHATAPRRRVQWKGRRVKRVLRPGTTVVFTPGERSTRTYKRVYDEVYGDEDLLEQANERLGEFAYGKRHKDMLALPLDEGNPTPSLKPVTLQQVLPALAPSEEKRGLKRESGDLAPTVQLMVPKRQRLEDVLEKMTVEPGLEPEVRVRPIKQVAPGLGVQTVDVQIPTTSSTSIATATEGMETQTSPVASAVADAAVQAVAAAASKTSTEVQTDPWMFRVSAPRRPRGSRKYGAASALLPEYALHPSIAPTPGYRGYTYRPRRRATTRRRTTTGTRRRRRRRQPVLAPISVRRVAREGGRTLVLPTARYHPSIV

SEQ ID NO: 176 - protein, 80 aa, Human adenovirus 5
MALTCRLRFPVPGFRGRMHRRRGMAGHGLTGGMRRAHHRRRRASHRRMRGGILPLLIPLIAAAIGAVPGIASVALQAQRH

SEQ ID NO: 177 - protein, 250 aa, Human adenovirus 5
MEDINFASLAPRHGSRPFMGNWQDIGTSNMSGGAFSWGSLWSGIKNFGSTVKNYGSKAWNSSTGQMLRDKLKEQNFQQKVVDGLASGISGVVDLANQAVQNKINSKLDPRPPVEEPPPAVETVSPEGRGEKRPRPDREETLVTQIDEPPSYEEALKQGLPTTRPIAPMATGVLGQHTPVTLDLPPPADTQQKPVLPGPTAVVVTRPSRASLRRAASGPRSLRPVASGNWQSTLNSIVGLGVQSLKRRRCF

SEQ ID NO: 178 - protein, 951 aa, Human adenovirus 5
ATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 179 - protein, 204 aa, Human adenovirus 5
MGSSEQELKAIVKDLGCGPYFLGTYDKRFPGFVSPHKLACAIVNTAGRETGGVHWMAFAWNPHSKTCYLFEPFGFSDQRLKQVYQFEYESLLRRSAIASSPDRCITLEKSTQSVQGPNSAACGLFCCMFLHAFANWPQTPMDHNPTMNLITGVPNSMLNSPQVQPTLRRNQEQLYSFLERHSPYFRSHSAQIRSATSFCHLKNM

SEQ ID NO: 180 - protein, 807 aa, Human adenovirus 5
MESVEKKDSLTAPSEFATTASTDAANAPTTFPVEAPPLEEEEVIIEQDPGFVSEDDEDRSVPTEDKKQDQDNAEANEEQVGRGDERHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLNEVPKIFEGLGRDEKRAANALQQENSENESHSGVLVELEGDNARLAVLKRSIEVTHFAYPALNLPPKVMSTVMSELIVRRAQPLERDANLQEQTEEGLPAVGDEQLARWLQTREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVWQQCLEECNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLNESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQQPRALASQDGTQKEAAAAAATHGRGGILGQSGRGGFGRGGGGHDGRLGEPRRGSFRGRRGVRRNTVTLGRIPLAGAPEIGNRFQHGYNLRSSGAAGTARSPTQP

SEQ ID NO: 181 - protein, 194 aa, Human adenovirus 5
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAGKSKQPPPLAQEQQQRQGYRSWRGHKNAIVACLQDCGGNISFARRFLLYHHGVAFPRNILHYYRHLYSPYCTGGSGSNSSGHTEAKATG

SEQ ID NO: 182 - protein, 227 aa, Human adenovirus 5
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAAHTAPAAAAATAAATQKQRRPDSKTLTKPKKSTAAAAAGGGALRLAPNEPVSTRELRNRIFPTLYAIFQQSRGQEQELKIKNRSLRSLTRSCLYHKSEDQLRRTLEDAEALFSKYCALTLKD

SEQ ID NO: 183 - protein, 227 aa, Human adenovirus 5
MSKEIPTPYMWSYQPQMGLAAGAAQDYSTRINYMSAGPHMISRVNGIRAHRNRILLEQAAITTTPRNNLNPRSWPAALVYQESPAPTTVVLPRDAQAEVQMTNSGAQLAGGFRHRVRSPGQGITHLTIRGRGIQLNDESVSSSLGLRPDGTFQIGGAGRPSFTPRQAILTLQTSSSEPRSGGIGTLQFIEEFVPSVYFNPFSGPPGHYPDQFIPNFDAVKDSADGYD

SEQ ID NO: 184 - protein, 581 aa, Human adenovirus 5
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 185 - protein, 449 aa, Human adenovirus 5
METRGRRPAALQHQQDQPQAHPGQRAARSAPLHRDPDYADEDPAPVERHDPGPSGRAPTTAVQRKPPQPAKRGDMLDRDAVEQVTELWDRLELLGQTLKSMPTADGLKPLKNFASLQELLSLGGERLLADLVRENMRVRDMLNEVAPLLRDDGSCSSLNYQLHPVIGVIYGPTGCGKSQLLRNLLSSQLISPTPETVFFIAPQVDMIPPSELKAWEMQICEGNYAPGPDGTIIPQSGTLRPRFVKMAYDDLILEHNYDVSDPRNIFAQAAARGPIAIIMDECMENLGGHKGVSKFFHAFPSKLHDKFPKCTGYTVLVVLHNMNPRRDMAGNIANLKIQSKMHLISPRMHPSQLNRFVNTYTKGLPLAISLLLKDIFRHHAQRSCYDWIIYNTTPQHEALQWCYLHPRDGLMPMYLNIQSHLYHVLEKIHRTLNDRDRWSRAYRARKTPK

SEQ ID NO: 186 - protein, 140 aa, Human adenovirus 5
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAV

SEQ ID NO: 187 - protein, 217 aa, Human adenovirus 5
MKIVGADGQEQEETDIPFRLWRKFAARRKLQYQSWEEGKEVLLNKLDRNLLTDFKAFAARFSSRPRPSKIFGTSLSEAISGEGNGQSGRGAARNHPRARTRCGATSPNHGGRVVPVPVAAASPGAPKKADEAAYRVRGRGRLITRRAGAAHTQPAAIDLGGGFGHCAQEEKEAPFSQARAPAITRGNRGQRGRKRRCGATNGGFQQPTGANQAWQRR

(19) United States
(12) Patent Application Publication
(10) Pub. No.: US 2018/0000926 A1
(43) Pub. Date: Jan. 4, 2018

(54) METHODS OF INDUCING AN IMMUNE RESPONSE TO HEPATITIS C VIRUS

(71) Applicants: Babita Agrawal, Edmonton (CA); Rakesh Kumar, Edmonton (CA); Shakti Singh, Edmonton (CA)

(72) Inventors: Babita Agrawal, Edmonton (CA); Rakesh Kumar, Edmonton (CA); Shakti Singh, Edmonton (CA)

(21) Appl. No.: 15/543,216

(22) PCT Filed: Jan. 15, 2016
(86) PCT No.: PCT/CA2016/000015

§ 371 Date: Jul. 12, 2017

Related U.S. Application Data
(60) Provisional application No. 62/103,919, filed on Jan. 15, 2015.

(51) Int. Cl. A61K 39/12; C12N 7/00; A61K 39/00

(52) U.S. Cl. CPC A61K 39/12; A61K 39/0011; C12N 7/00; A61K 2039/575; A61K 2039/572; A61K 2039/54; A61K 2039/53; A61K 2039/5256; A61K 2039/5154; C12N 2770/24234; C12N 2770/24271; A61K 2039/57

(57) ABSTRACT

The present disclosure provides methods for inducing an immune response to hepatitis C virus (HCV) in an individual. The present disclosure provides methods for treating an HCV infection in an individual.

### CROSS-REFERENCE

This application claims the benefit of U.S. Provisional Patent Application No. 62/103,919, filed Jan. 15, 2015, which application is incorporated herein by reference in its entirety.

### INTRODUCTION

Adenoviruses are diverse group of DNA viruses, which include both primate and non-primate adenoviruses, and are classified under family Adenoviridae. Adenoviruses are 90-100 nm size, non-enveloped double stranded DNA viruses, which have an icoshedral nucleo-capsid. Human adenoviruses have been classified in to 51 serotypes and 6 sub groups (A-F) on the basis of neutralization with specific anti-sera.

Recombinant adenoviruses carrying a foreign transgene are intensively being tested both as prophylactic and therapeutic vaccine for a number of pathogens in human clinical trials. They have proven to be safe, efficient and excellent vehicles for transferring vaccine antigens and eliciting immune responses against the transgene antigen in many non-human animal and human clinical testing.

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

### LITERATURE

U.S. Pat. No. 8,871,515; U.S. Pat. No. 8,142,794

### SUMMARY

The present disclosure provides methods for inducing an immune response to hepatitis C virus (HCV) in an individual. The present disclosure provides methods for treating an HCV infection in an individual.

The present disclosure provides a method of inducing an immune response in an individual to an HCV protein, the method comprising administering to the individual an effective amount of an immunogenic composition comprising an adenoviral nucleic acid or an adenovirus polypeptide. In some cases, the adenoviral nucleic acid or adenovirus polypeptide is administered via an oral, intranasal, subcutaneous, transdermal, intratracheal, rectal, intramuscular or parenteral route of administration. In some cases, the adenoviral nucleic acid or adenovirus polypeptide is administered multiple times. In some cases, the immune response comprises a humoral and/or a cellular immune response. In some cases, the adenoviral nucleic acid is a full-length adenovirus nucleic acid or an adenovirus nucleic acid comprising a deletion. In some cases, the adenoviral nucleic acid does not encode a non-adenovirus polypeptide. In some cases, the adenoviral nucleic acid comprises a nucleotide sequence encoding one or more HCV polypeptides. In some cases, the adenoviral nucleic acid comprises a nucleotide sequence encoding an antigen associated with a pathogen other than HCV or comprises a nucleotide sequence encoding a cancer-associated antigen. In some cases, the adenoviral nucleic acid comprises a nucleotide sequence associated with an immunostimulatory or immunomodulatory sequence. In some cases, the method comprises simultaneously administering a non-recombinant adenovirus. In some cases, the method comprises administering a structural or a non-structural HCV polypeptide or a nucleic acid comprising a nucleotide sequence encoding the structural or non-structural HCV polypeptide. In some cases, the HCV polypeptide is one or more of E1, E2, F, core, P7, NS2, NS3, NS4 and NS5. In some cases, the structural or non-structural HCV antigen is administered before the adenovirus nucleic acid or the adenovirus polypeptide. In some cases, the structural or non-structural HCV antigen is administered after the adenovirus nucleic acid or the adenovirus polypeptide. In some cases, the immunogenic composition comprises an adjuvant. In some cases, the composition comprises a cytokine and/or an antibody.

The present disclosure provides a method of inducing an immune response in an individual to a hepatitis C virus antigen, the method comprising: a) obtaining dendritic cells (DCs) from the individual; b) genetically modifying the DCs to express one or more adenoviral proteins; and c) administering the genetically modified DCs to the individual.

The present disclosure provides a method of inducing an immune response in an individual to a hepatitis C virus antigen, the method comprising: a) obtaining DCs from the individual; b) infecting the DCs with replication competent adenovirus or replication-defective adenovirus; and c) administering the infected DCs to the individual.

The present disclosure provides a method of inducing an immune response in an individual to a hepatitis C virus antigen, the method comprising: a) obtaining DCs from the individual; b) introducing one or more adenoviral proteins, or nucleic acids encoding one or more adenoviral proteins, into the DCs, thereby generating adenoviral protein-expressing DCs; and c) administering the adenoviral protein-expressing DCs to the individual.

The present disclosure provides a method of treating a hepatitis C virus (HCV) infection in an individual, the method comprising inducing an immune response to one or more HCV antigens in the individual, wherein said inducing comprises a method as disclosed above or elsewhere herein. In some cases, the method comprises administering to the individual an effective amount of at least a second therapeutic agent that treats an HCV infection. In some cases, the HCV-infected individual is a treatment-naïve individual. In some cases, the HCV-infected individual failed a prior treatment for HCV infection. The HCV can be an HCV of any genotype or subtype.

### BRIEF DESCRIPTION OF THE DRAWINGS

FIGS. 1A and 1B. FIG. 1A shows an electronic picture of agarose electrophoresis of PCR amplified products of genetic material prepared from various adenoviral vector stocks using HCV gene specific primers. This confirms that the adenoviral vector stocks used in experiments to immunize mice were free from contamination of adenoviral vectors encoding HCV antigens. First panel shows the DNA ladder, followed by agarose gel electrophoresis of PCR products obtained with HCV Core, F, NS3, NS4, NS5a and NS5b specific primers. HEK lysate supernatant and rAd-HCV were taken as negative and positive controls. FIG. 1B shows the detection of cross-reactive binding of anti-core and anti-NS3 antibodies to mouse quadriceps muscles upon immunization with adenoviral vector. Mice were immunized with 2×10⁷ pfu replication deficient adenoviruses containing HCV-core, NS3 or empty adenoviral vector (Ad) intramuscularly. Twelve, 24 and 48 hours after immunization, quadricep muscle cells were isolated and cut into thin slices. Immunohistochemistry was done to confirm expression of core and NS3 protein and cross-reactive binding of anti-core and anti-NS3 Mabs. Samples from PBS control mice were obtained 12 hours after inoculation and were stained with anti-core antibody (A, left panel) and anti-NS3 antibody (A, right panel). Samples from mice immunized with adenoviral vector alone (Ad), recombinant adenoviral vectors containing core (rAd-Core) or NS3 (rAd-NS3) antigens and stained with anti-core and anti-NS3 antibody after 12 hours (B), 24 hours (C) and 48 hours (D). At each time point, rAd-core and rAd-NS3 immunized mice were stained with anti-core or anti-NS3 antibody, respectively, as positive controls.

FIGS. 2A-C demonstrate cross-reactive cellular immune responses generated against HCV core protein and a pool of 5 high homology peptides (Core Pool: 5, 14, 16, 17 & 27) in mice immunized with adenoviral vector (Ad) in the absence or presence of toll-like receptor agonist poly I:C (Ad+IC) or resiquimod (Ad+RQ) as adjuvants. Groups of five C57bl/6 female mice were immunized twice (at 14 days interval) with 2×10⁷ pfu/mouse adenoviral vector intramuscularly in quadriceps muscles in a total volume of 150 microlitre/mouse. A recombinant Ad vector expressing NS3 coding region (rAd-NS3) was also used to demonstrate adenoviral vector inducing cross-reactive immunity. PBS immunized mice were used as negative controls. Eight days after second immunization, mice were euthanized and spleen and inguinal lymph nodes were collected. Enriched T cells (4×10⁵/well) from spleens and lymph nodes were cultured with irradiated syngeneic spleen cells as APCs (4×10⁵/well) and recombinant HCV core protein or HCV core derived synthetic peptides pool (5 μg/ml) for four days. Culture supernatants were collected for cytokine analysis and proliferation of T cells was examined by ³H thymidine incorporation assay. All data represent mean±standard deviations of triplicate wells. The experiments were repeated 3-5 times and representative data is presented. FIG. 2A: proliferation of spleen or lymph nodes derived T cells upon stimulation with recombinant core protein antigen or peptide pool. FIGS. 2B and 2C: induction of IFN-γ and IL-10 (picogram/ml) in ex vivo antigen stimulated T cell culture supernatants.

FIGS. 3A-C show cross-reactive cellular immune responses against HCV NS3 protein and a pool of 5 high homology peptides (NS3 Pool: 5, 6, 8, 15 & 17) in mice immunized with adenoviral vector (Ad) in the absence or presence of toll-like receptor agonists [poly I:C (Ad+IC) or resiquimod (Ad+RQ)], rAd-NS3 or PBS. The immunization and ex vivo T cell culture protocols were similar as described in FIG. 2. FIG. 3A: proliferation of spleen and lymph node T cells upon stimulation with HCV-NS3 protein or peptides pool at 5 μg/ml concentration. FIGS. 3B and 3C: induction of IFN-γ and IL-10 (picogram/ml) in ex vivo antigen stimulated T cell culture supernatants.

FIGS. 4A-C show cross-reactive cellular immune responses against HCV NS5 protein and a pool of 5 high homology peptides (NS5a Pool: 6, 24 and NS5b pool: 5, 19 & 27) in mice immunized with adenoviral vector (Ad) in the absence or presence of toll-like receptor agonists [poly I:C (Ad+IC) or resiquimod (Ad+RQ)], rAd-NS3 or PBS. The immunization and ex vivo T cell culture protocols were similar to FIG. 2. FIG. 4A: proliferation of spleen or lymph node T cells upon stimulation with HCV-NS5 protein antigen or peptide pool at 5 μg/ml concentration. FIGS. 4B and 4C: induction of IFN-γ and IL-10 (picogram/ml) in ex vivo antigen stimulated T cell culture supernatants.

FIGS. 5A-H show cross-reactive antibody (IgG and IgG1) responses against HCV core, NS3, NS4 and NS5 proteins in serum of mice immunized with adenoviral vector (Ad) in the absence or presence of toll-like receptor agonists [poly I:C (Ad+IC) or resiquimod (Ad+RQ)], rAd-NS3 or PBS as described in FIG. 2. Sera from mice of the same group were pooled. For the detection of antibodies against HCV core, NS3, NS4 or NS5, 96-well plates were coated with specific recombinant HCV protein (1 μg/ml in 1×PBS) and incubated overnight at 4° C. Plates were washed and blocked with 1% BSA followed by addition of diluted pooled serum in duplicate. The plates were incubated for 2 hours, washed with 1×PBST, and added with secondary antibody (anti-mouse IgG AP labeled) (Southern Biotech, Alabama, USA) and color was developed with PNPP substrate. Absorbance was read using a FluoStar ELISA Reader. Graphs A-H represent the absorbance value at different dilution of serum. Means±SD of triplicate values are shown.

FIG. 6A shows HCV specific and cross-reactive proliferation of spleen T cells obtained from mice immunized twice with adenoviral vector (Ad) via different routes (intramuscular, intranasal and oral) upon stimulation with HCV protein antigens (core, NS3, NS4 and NS5) or high homology peptide pools from respective HCV antigens.

FIG. 6B shows amounts of IFN-γ production (picogram/ml) in culture supernatants from T cell proliferation assay. Solid dark bars represent Ad immunized mice and white bars PBS immunized mice. No bars in FIG. 6B represent cytokines below detection levels.

FIG. 6C shows antigen specific proliferation of spleen and/or lymph node T cells from mice immunized once intramuscularly (i.m.) or intranasally (i.n.) with different doses of Ad vector (Ad) or rAd-NS3, upon in vitro stimulation with various HCV protein antigens (core, NS3, NS4, NS5). I, II. Single intramuscular immunization with Ad or rAd-NS3 (0.5×10⁶ pfu/mouse); III, IV. Single intramuscular immunization with Ad or rAd-NS3 (1.0×10⁶ pfu/mouse); V. Single intramuscular immunization with Ad or rAd-NS3 (2.0×10⁷ pfu/mouse); and VI. Single intranasal immunization with Ad or rAd-NS3 (1×10⁷ pfu/mouse).

FIGS. 7A-C. FIG. 7A shows cross-reactive proliferation of spleen T cells obtained from adenoviral vector (Ad) immunized mice (twice intramuscularly), upon in vitro stimulation with individual HCV core peptides at 5 μg/ml (listed in TABLE 1; FIG. 18) or core protein at 1 μg/ml. The immunization and ex vivo T cell culture protocols used were similar to FIG. 2. FIGS. 7B and 7C: induction of IFN-γ and IL-10 (picogram/ml) in ex vivo HCV core antigen stimulated T cell culture supernatants. Solid dark bars represent Ad immunized mice and white bars represent PBS immunized mice.

FIGS. 8A-C. FIG. 8A shows cross-reactive proliferation of spleen T cells obtained from adenoviral vector (Ad) immunized mice (twice intramuscularly), upon in vitro stimulation with 5 μg/ml of individual HCV F peptides (listed in TABLE 2; FIG. 19). The immunization and ex vivo T cell culture protocols were similar to FIG. 2. FIGS. 8B and 8C show induction of IFN-γ and IL-10 (picogram/ml) in ex vivo HCV F peptides stimulated T cell culture supernatants. Solid dark bars represent Ad immunized mice and white bars show PBS immunized mice.

FIGS. 9A-C. FIG. 9A shows cross-reactive proliferation of spleen T cells obtained from adenoviral vector (Ad) immunized mice (twice intramuscularly), upon in vitro stimulation with 5 μg/ml of HCV NS3 peptides (listed in TABLE 3; FIG. 20) or NS3 protein (1 μg/ml). The immunization and ex vivo T cell culture protocols were similar to FIG. 2. FIGS. 9B and 9C show induction of IFN-γ and IL-10 (picogram/ml) in ex vivo HCV NS3 antigen stimulated T cell culture supernatants. Solid dark bars represent Ad immunized mice and white bars show PBS immunized mice.

FIGS. 10A-C show cross-reactive cellular immune responses against HCV NS4 protein or peptides in mice immunized with adenoviral vector (Ad) upon two intramuscular immunizations. FIG. 10A: splenic T cell proliferation upon stimulation with individual HCV NS4 peptides at 5 μg/ml (TABLE 4; FIG. 21) or HCV NS4 protein antigen at 1 μg/ml concentrations. FIGS. 10B and 10C: IFN-γ and IL-10 concentration (picogram/ml) in spleen T cell culture supernatants from T cell proliferation.

FIGS. 11A-C. FIG. 11A shows cross-reactive proliferation of spleen T cells from adenoviral vector (Ad) immunized mice, upon in vitro stimulation with 5 μg/ml of individual HCV NS5a peptides (listed in TABLE 5a; FIG. 22) or HCV NS5 protein antigen at 1 μg/ml. The immunization and ex vivo T cell culture protocols were similar to FIG. 2. FIGS. 11B and 11C: induction of IFN-γ and IL-10 (picogram/ml) in ex vivo antigen stimulated T cell culture supernatants. Solid dark bars represent Ad immunized mice and white bars show PBS immunized mice.

FIGS. 12A-C. FIG. 12A shows cross-reactive proliferation of spleen T cells from adenoviral vector (Ad) immunized mice, upon in vitro stimulation with 5 μg/ml of HCV NSSB peptides (listed in TABLE 5B; FIG. 23) or HCV NS5 protein antigen at 1 μg/ml. The immunization and ex vivo T cell culture protocols were similar to FIG. 2. FIGS. 12B and 12C: induction of IFN-γ and IL-10 (picogram/ml) in ex vivo antigen stimulated T cell culture supernatants. Solid dark bars represent Ad immunized mice and white bars show PBS immunized mice.

FIGS. 13A-D. FIGS. 13A and 13B show proliferation of spleen derived CD4⁺ and CD8⁺ T cells harvested from adenoviral vector (Ad) immunized mice in response to various HCV protein antigens by CFSE dilution assay. The immunization protocol was similar as described in FIG. 2. Spleen T cells were stained with CFSE, and incubated with irradiated syngeneic spleen cells as APCs and various HCV antigens (core, NS3, NS4 and NS5 at 5 μg/ml concentration) for 4 days. Loss of CFSE due to cell division was compared with the T cells incubated in the absence of added antigen and represented as histograms. Shift of peak of CFSE T cells towards left was considered as indication of level of antigen specific T cell proliferation. FIGS. 13 C and D show intracellular cytokine (IFN-γ and IL-10) expression of CD4⁺ and CD8⁺ T cells obtained from Ad vector or PBS immunized mice and stimulated with various HCV proteins (Core, NS3, NS4 and NS5) for four days. The data represent the percentage of IFN-γ and IL-10 expressing CD4⁺ T cells and CD8⁺ T cells.

FIGS. 14A-D. 14A and B show proliferation of CD4⁺ and CD8⁺ T cells harvested from Ad immunized mice in response to various HCV specific synthetic peptides by CFSE dilution assay. The immunization protocol used was similar to FIG. 2. Spleen T cells were stained with CFSE, and incubated with irradiated syngeneic spleen cells as APCs and various representative peptides (showing high homologies to Ad proteins) from HCV proteins Core (peptide #6), NS3 (peptide #8), NS4 (peptide #4) and NS5 (peptide #19) at 5 μg/ml. After 4 days of incubation, loss of CFSE due to cell division was compared with the T cells incubated in the absence of peptide antigen and represented as histograms. Shift in peak of CFSE⁺ T cells towards left was considered as indication of antigen specific T cell proliferation. FIGS. 14C and D show intracellular cytokine (IFN-γ and IL-10) expression of CD4⁺ and CD8⁺ T cells from spleens of mice immunized with Ad vector or PBS and cultured ex vivo with representative HCV derived synthetic peptides at 5 μg/ml concentration. The data represent the percentage of IFN-γ and IL-10 expressing CD4⁺ T cells and CD8⁺ T cells in response to various representative peptides of HCV proteins.

FIG. 15 shows the cytotoxic activity of effector T cells harvested from Ad vector immunized mice against HCV peptides loaded CFSE stained EL4 targets. Spleen T cells harvested from Ad immunized mice were stimulated in vitro with the HCV protein antigens Core, NS3, NS4 or NS5 at 5 μg/ml concentration for 4 days. The target EL4 cells were incubated with corresponding HCV peptides (Core peptides: 2, 14, 17, 25, 27, 28, 32; NS3 peptides: 8, 10; NS4 peptides: 3, 4, 8; and NS5 peptides: 1a, 2a, 16a, 20a, 5b, 19b, 23b, 39b; or All: a mixture of the above peptides from core, NS3, NS4 and NS5) and peptide loaded EL4 cells were cultured with effectors at 10:1 (effectors:target) ratio for 4-5 hours. CFSE labeled live targets were quantified by flow cytometry and subtracted from background CFSE labeled targets to get numbers of killed targets. Empty (no peptide loaded) EL4 targets were used as a negative control.

FIGS. 16A and 16B. FIG. 16A shows the stimulation of cross-reactive HCV specific CD4⁺ and CD8⁺ T cell proliferative responses (by CFSE dilution assay) against a representative HCV antigen (NS5) upon immunization with adenoviral vector (Ad) infected bone marrow derived dendritic cells (DCs). Group immunized with bone marrow DCs (treated with media) only served as control. FIG. 16B: demonstrates increased expression of granzyme B on cross-reactive CD8⁺ T cells in response to HCV NS5 antigen.

FIGS. 17A and 17B. FIG. 17 A shows that immunization of mice with adenoviral vector (Ad) leads to reduction in the titer of infectious vaccinia-HCV chimeric virus in mice. Groups of mice were immunized with Ad vector (2×10⁷ pfu/mouse) (n=11), Ad vector in presence of poly I:C adjuvant (n=5), and PBS control (n=4). Eight days after two intramuscular immunizations (14 days apart), mice were challenged intraperitoneally with infectious chimeric Vac-HCV (NS3-NS4-NS5) (1×10⁷ PFU/mouse), and ovaries were harvested 5 days after challenge. Viral loads in each mouse ovary were determined by plaque assay using TK-1 cells. FIG. 17 B demonstrates that immunization of mice with adenoviral vector (Ad) leads to reduction in the titer of infectious vaccinia-HCV (Vac-Core-NS3) chimeric virus in mice but not of the wild-type-Vaccinia (WT-Vac). Groups (n=5) of mice were immunized with Ad vector or HEK cell lysate (control). Eight days after two intramuscular immunizations (14 days apart), mice were challenged with chimeric vaccinia (Vac-Core-NS3) or wild-type vaccinia (WT-Vac) (1×10⁷ PFU/mouse) intraperitoneally, and ovaries were harvested 5 days after challenge. Viral loads in each mouse ovary were determined by plaque assay using TK-1 cells.

FIG. 18 provides TABLE 1, which shows the score of amino acid sequence homology between adenoviral vector (Ad) proteins and peptide epitopes of HCV Core protein, and also number of epitope regions in Ad proteins, which show homology (>25) with the HCV Core peptides/epitopes (S. No. 1-45 correspond to SEQ ID NOs:1-45).

FIG. 19 provides TABLE 2, which illustrates the score of amino acid sequence homology between Ad proteins and peptide epitopes of HCV frame shift protein (F), and also number of epitope regions in Ad proteins, which show homology (>25) with the HCV F peptides/epitopes (S. No. 1-16 correspond to SEQ ID NOs:46-61).

FIG. 20 provides TABLE 3, which shows the score of amino acid sequence homology between Ad proteins and selected peptide epitopes of HCV NS3 protein, and also number of epitope regions in Ad proteins, which show homology (>25) with the HCV NS3 peptides/epitopes (S. No. 1-11 correspond to SEQ ID NOs:62-72).

FIG. 21 provides TABLE 4, which shows the score of amino acid sequence homology between Ad proteins and peptide epitopes of HCV NS4 protein and also number of epitope regions in Ad proteins, which show homology with the HCV NS4 peptides/epitopes (first set of S. No. 1-4 correspond to SEQ ID NOs:73-76; second set of S. No. 1-16 correspond to SEQ ID NOs:77-92).

FIGS. 22 and 23 provide TABLE 5a and 5b, which show the score of amino acid sequence homology between Ad proteins and peptide epitopes of HCV NS5a and NS5b proteins, respectively. Tables 5a and 5b also summarize the number of epitope regions in Ad proteins, which show homology (>25) with the HCV NS5a and 5b peptides/epitopes (Table 5a: S. No. 1-29 correspond to SEQ ID NOs:93-121; Table 5b: S. No. 1-39 correspond to SEQ ID NOs:121-160).

FIG. 24 provides TABLE 6, which lists the Ad5 vector proteins whose amino acid sequences were aligned with HCV synthetic peptide sequence.

FIGS. 25A-I provides TABLE 7, which provides amino acid sequences of adenoviral proteins (SEQ ID NOs:161-187).

FIG. 26 demonstrates cross-reactive T cell responses generated against HIV-gp120 and HCV core, NS3, NS4 and NS5 protein in mice immunized with recombinant adenoviral vector expressing HIV-nef antigen (rAd-nef) in the absence or presence of toll-like receptor agonist poly I:C (rAd-nef+Poly I:C) as adjuvant. Groups of five C57bl/6 male mice were immunized twice (at 14 days interval) with 2×10⁷ pfu/mouse adenoviral vector intranasally in each nostril (15 ul/nostril) in a total volume of 30 microliter/mouse. PBS immunized mice were used as negative controls. Eight days after second immunization, mice were euthanized and spleen was collected. Enriched T cells (4×10⁵/well) from spleens were cultured with irradiated syngeneic spleen cells as APCs (4×10⁵/well) and recombinant HIV gp-120, and HCV core, NS3, NS4 and NS5 proteins for four days. Proliferation of T cells was examined by ³H thymidine incorporation assay. All data represent mean±standard deviations of triplicate wells. Proliferation of spleen derived T cells upon stimulation with recombinant HCV (core, NS3, NS4 and NS5) protein antigens and HIV-nef protein is shown as Avg±S.D.

FIG. 27 demonstrates cross-reactive T cell responses generated against HCV core, NS3 and NS4 protein antigens in mice immunized with recombinant adenoviral vector expressing mycobacterial antigen 85B (rAd-Ag85B) or Ad vector. Groups of five C57bl/6 male mice were immunized twice (at 14 days interval) with 2×10⁷ pfu/mouse adenoviral vector intramuscularly in quadricep muscles in a total volume of 150 microlitre/mouse. Eight days after second immunization, mice were euthanized and spleen was collected. Enriched T cells (4×10⁵/well) from spleens were cultured with irradiated syngeneic spleen cells as APCs (4×10⁵/well) and recombinant HCV (core, NS3 and NS4) protein antigens for four days. Proliferation of T cells was examined by ³H thymidine incorporation assay. All data represent mean±standard deviations of triplicate wells. Proliferation of spleen derived T cells upon stimulation with recombinant HCV (core, NS3 and NS4) protein antigens and sonicated mycobacteria is shown as Avg±S.D.

FIG. 28 demonstrates that priming of mice with adenoviral vector (Ad, 2×10⁷ pfu/mouse, intramuscular) and boosting with pool of HCV-NS3 peptides with heat-killed Caulobacter crescentus (HKCC) intranasally leads to reduction in the titer of infectious vaccinia-HCV (Vac-Core-NS3) chimeric virus in mice. Groups (n=5) of female mice were immunized with Ad (i.m.) followed by a boost with a mixture of HCV NS3 peptides and HKCC (i.n) or PBS. Eight days after second immunization (14 days apart), mice were challenged with chimeric vaccinia (Vac-Core-NS3) (1×10⁷ PFU/mouse) intraperitoneally, and ovaries were harvested 5 days after challenge. Viral loads in individual mouse ovaries were determined by plaque assay using TK-1 cells.

FIG. 29 provides TABLE 8, which shows the score of amino acid sequence homology between peptide epitopes of HCV core protein and Chimp Ad25 proteins (S. No. 1-45 corresponds to SEQ ID NOs:1-45).

### DEFINITIONS

The terms “Adenovirus” and “Adenoviral vector” as used herein include any and all viruses that may be categorized as an Adenovirus, including any Adenovirus that infects a human or an animal, including all groups, subgroups, and serotypes. Thus, as used herein, “Adenovirus” and “Adenovirus vector” refer to the virus itself or derivatives thereof and cover all serotypes and subtypes and both naturally occurring and recombinant forms. In one embodiment, such Adenoviruses infect human cells. Such Adenoviruses may be wildtype or may be modified in various ways known in the art or as disclosed herein. Such modifications include modifications to the Adenovirus genome that is packaged in the particle. Such modifications include deletions known in the art, such as deletions in one or more of the E1a, E1b, E2a, E2b, E3, or E4 coding regions.

By “HCV” herein is meant any one of a number of different genotypes and isolates of hepatitis C virus. Representative HCV genotypes and isolates include: H77, the “Chiron” isolate, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6 and type 6a.

As used herein, “subject” or “individual” or “patient” refers to any subject for whom or which therapy is desired, and generally refers to the recipient of the therapy to be practiced according to the invention. The subject can be any vertebrate, but will typically be a mammal. If a mammal, the subject will in many embodiments be a human, but may also be a domestic livestock, a field animal such as a horse, laboratory subject or pet animal.

The term “effective amount” or “therapeutically effective amount” means a dosage sufficient to provide for treatment for the disease state being treated or to otherwise provide the desired effect (e.g., reduction of viral load). The precise dosage will vary according to a variety of factors such as subject-dependent variables (e.g., age, immune system health, etc.), the disease (e.g., the particular viral strain), and the treatment being effected. In the case of treatment of HCV infection, an “effective amount” can be considered that amount sufficient to reduce the HCV viral load in a subject, as described in more detail below.

The terms “treat,” “treating,” “treatment” and the like are used interchangeably herein and mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed the disease. “Treating” as used herein covers treating a disease in a vertebrate, e.g., a mammal, e.g., a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an adenoviral nucleic acid” includes a plurality of such nucleic acids and reference to “the HCV polypeptide” includes reference to one or more HCV polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method of inducing an immune response to one or more hepatitis C virus (HCV) antigens in an individual in need thereof. The method generally involves administering to the individual an immunogenic adenovirus composition, where the adenovirus nucleic acid or adenovirus polypeptide present in the immunogenic adenovirus composition induces an immune response in the individual to one or more HCV antigens. In some cases, the adenovirus a wild-type adenovirus. In some cases, the adenovirus is a recombinant adenovirus. The present disclosure provides a method of treating an HCV infection in an individual, the method comprising inducing an immune response to one or more HCV antigens in the individual.

### Methods of Inducing an Immune Response to HCV

The present disclosure provides a method of inducing an immune response to one or more HCV antigens in an individual in need thereof. The method generally involves administering to the individual an immunogenic adenovirus composition, where the adenovirus nucleic acid or adenovirus polypeptide present in the immunogenic adenovirus composition induces an immune response in the individual to one or more HCV antigens. In some cases, the adenovirus a wild-type adenovirus. In some cases, the adenovirus is a recombinant adenovirus. In some embodiments, the individual is not infected with HCV.

As noted above, a method of the present disclosure for inducing an immune response to one or more HCV antigens involves administering to an individual in need thereof an effective amount of an adenovirus composition. An “adenovirus composition” can include an adenovirus nucleic acid or an adenovirus polypeptide.

In some cases, the adenovirus composition used in a method of the present disclosure for inducing an immune response in an individual to one or more HCV antigens comprises an adenovirus polypeptide. In some cases, the adenovirus polypeptide is a fusion protein; e.g., a fusion protein comprising an adenovirus polypeptide and a non-adenovirus polypeptide, where the non-adenovirus polypeptide is referred to as a “fusion partner.” Suitable fusion partners include, e.g., carrier polypeptides (e.g., keyhole limpet hemocyanin; hepatitis B virus core antigen; and the like); a polypeptide associated with (or produced by) a bacterial pathogen; a polypeptide associated with (or produced by) a viral pathogen; a tumor-associated antigen; and the like.

In some cases, an adenovirus suitable for use in a method of the present disclosure is a recombinant adenovirus. In some cases, recombinant adenovirus does not include any non-adenovirus sequences. In some cases, recombinant adenovirus lacks sequences present in a naturally-occurring adenovirus; e.g., the recombinant adenovirus comprises a deletion relative to a naturally-occurring adenovirus. In some cases, recombinant adenovirus lacks sequences present in a naturally-occurring adenovirus; and includes non-adenovirus sequences.

In some cases, recombinant adenovirus suitable for use in a method of the present disclosure lacks sequences present in a naturally-occurring adenovirus; e.g., the recombinant adenovirus comprises a deletion relative to a naturally-occurring adenovirus. A recombinant adenovirus can lack from 1 nucleotide (nt) to 1 kb relative to a naturally-occurring adenovirus. A recombinant adenovirus suitable for use in a method of the present disclosure can have a deletion of an all or part of an adenovirus gene. For example, a recombinant adenovirus suitable for use in a method of the present disclosure can be deleted for all or part of the adenovirus E3 gene. As another example, a recombinant adenovirus suitable for use in a method of the present disclosure can be deleted for all or part of the adenovirus E1 gene. As another example, a recombinant adenovirus suitable for use in a method of the present disclosure can be deleted for all or part of the adenovirus E1 gene and can be deleted for all or part of the adenovirus E3 gene. In some cases, an adenovirus composition for use in a method of the present disclosure is a replication-defective adenovirus.

Adenovirus in an immunogenic adenovirus composition for use in a method of the present disclosure can be from any of a variety of sources. Suitable sources include, but are not limited to, bovine adenovirus, a canine adenovirus, non-human primate adenovirus, gorilla adenovirus, a chicken adenovirus, a porcine adenovirus, a swine adenovirus, an adeno associated virus-dependent adenovirus, human adenovirus, and any serotype or subtype of an adenovirus. For example, where the adenovirus is a human adenovirus, any of 57 different serotypes from 7 subtypes of human adenoviruses can be used. As another example, where the adenovirus is a Chimp adenovirus, any of a variety of different serotypes from three different groups (B,C,E) of Chimp adenoviruses can be used. In some cases, adenovirus vector in an immunogenic adenovirus composition for use in a method of the present disclosure can be purified or partially purified. In some cases, an immunogenic adenovirus composition for use in a method of the present disclosure comprises adenovirus from different species and/or adenovirus of more than one serotype. In some cases, subtypes and serotypes of different species of adenoviruses can be used sequentially for repeated immunizations to avoid generation of neutralizing immunity.

In some cases, an adenovirus suitable for use in a method of the present disclosure is mutated or genetically engineered.

In some cases, chimeric adenovirus containing one or more sections from two or more different adenoviruses (from interspecies or intraspecies Ad viruses) can be used. Adenoviruses can also be constructed by modifying the backbone of one of the adenoviruses (e.g., one or more hypervariable or conserved regions, capsid, host cell receptor binding domains, knob, shaft, hexon and/or fiber proteins). One or more of these regions may be deleted, mutated, replaced by a linker or replaced by another region of a different virus or adenovirus. The fiber, knob or shaft can also be modified to give specific targeting ability to adenovirus vectors (e.g., dendritic cell (DC) targeting, hepatocyte targeting, smooth muscle targeting, fibroblast targeting etc.).

In some cases, an adenovirus can be modified to facilitate uptake and/or target into cells or tissues of interest.

Adenovirus vectors that are modified and optimized can allow for a significant reduction in therapeutic or prophylactic dose resulting in reduced toxicity.

In some cases, the adenovirus used in a method of the present disclosure is a recombinant adenovirus expressing an immunostimulatory or an immunomodulatory sequence that are heterologous to the virus e.g., TLR agonists (such as polyI:C, CpG, flagellin), double stranded RNA adjuvants, NOD like receptor agonists, agonists, T helper peptide epitopes/proteins (e.g., Tetanus toxoid, HBV core (human or woodchuck), Heat shock proteins), cytokines (e.g., IL-2, IL-12, GM-CSF, IL-15, IFN-g etc.), chemokines, adapter proteins involved in innate immune signaling (e.g., Ewing's Sarcoma related transcript-2, EAT-2), peptide mimetics, costimulatory molecules (e.g., CD40L), antibodies to coinhibitory molecules (e.g., anti-CTLA-4, anti-PD-1);

In some cases, the adenovirus used in a method of the present disclosure does not include nucleotide sequences encoding non-adenoviral proteins. In some cases, the adenovirus used in a method of the present disclosure includes nucleotide sequences encoding an HCV peptide listed in Tables 1-5, or a fragment of an HCV peptide listed in Tables 1-5.

Recombinant vectors can be produced, which vectors comprise nucleotide sequences encoding one or more adenoviral proteins or fragments thereof. In some cases, a method of the present disclosure for inducing an immune response in an individual to one or more HCV antigens comprises administering to the individual a composition comprising a recombinant bacterial, yeast, or viral vector comprising nucleotide sequences encoding one or more adenoviral proteins. A recombinant vector can be a viral vector such as adeno-associated virus, lentivirus, herpes virus, poxvirus, canarypox virus, vesicular stomatitis virus, alpha virus, measles virus, papaya mosaic virus, cytomegalovoirus, modified vaccinia Ankara virus MVA, polio virus, Marba virus etc.), bacterial vector vaccines (such as Salmonella, Shigella, E. coli, Lactococcus lactis, Listeria sp., Lactobacillus sp.), fungal vectors (such as heat killed recombinant Saccharomyces yeast), plant viruses, virus-like particles (VLPs), virosomes, synthetic vaccine particles, synthetic biomimetic supramolecular biovectors, depathogenized viral/bacterial strains (such as NIBRG14 from H5N1). The vector could be in the form of live wild-type, non-replicative, mutated, modified, defective or attenuated. The vectors could be from human, animal, plant or prokaryote origin and in any effective amount.

In some cases, the recombinant bacterial, yeast or viral vectors can include, in addition to adenoviral nucleotide sequences, an immunostimulatory or an immunomodulatory sequence that are heterologous to the virus e.g., TLR agonists (such as polyI:C, CpG, flagellin), double stranded RNA adjuvants, NOD like receptor agonists, agonists, T helper peptide epitopes/proteins (e.g., Tetanus toxoid, HBV core (human or woodchuck), Heat shock proteins), cytokines (e.g., IL-2, IL-12, GM-CSF, IL-15, IFN-γ, etc.), chemokines, adapter proteins involved in innate immune signaling (e.g., Ewing's Sarcoma related transcript-2, EAT-2), peptide mimetics, costimulatory molecules (e.g., CD40L), antibodies to coinhibitory molecules (e.g., anti-CTLA-4, anti-PD-1).

In some cases, a prime-boost immunization regimen is carried out. The prime boost regimen can include the following in any order: where a first immunogenic composition includes structural HCV antigens (E1 and/or E2) as recombinant protein, glycoprotein, polypeptide, fusion protein, DNA or recombinant vector (viral, bacterial or fungal) to induce cross reactive neutralizing antibodies; and a second immunogenic composition includes adenovirus (Ad) or recombinant adenovirus (rAd) containing various HCV antigens to induce cellular and humoral immune responses against multiple structural (e.g., core, F p7) and non structural antigens (e.g., NS2, NS3, NS4A, NS4B, NS5A, NS5B or combinations thereof) of HCV. The nucleotide sequences encoding the antigens can be from different genotypes (1-7) or subtypes of HCV. The prime boost regimens can include recombinant proteins, synthetic proteins, or peptide antigens along with adenovirus or recombinant adenovirus, with or without adjuvants.

Thus, in some cases, a method of the present disclosure of inducing an immune response in an individual to one or more HCV antigens comprises: a) administering, at a first time, to the individual HCV structural antigens E1 and/or E2; and b) administering, at a second time, to the individual an adenovirus, as described above. In some cases, a method of the present disclosure of inducing an immune response in an individual to one or more HCV antigens comprises: a) administering, at a first time, to the individual an adenovirus, as described above; b) administering, at a second time, to the individual HCV structural antigens E1 and/or E2. The first time and the second time can be separated from one another by 1 hour to 1 year, e.g., from 1 hour to 12 hours, from 12 hours to 24 hours, from 2 days to 1 week, from 1 week to 1 month, from 1 month to 3 months, from 3 months to 6 months, or from 6 months to 1 year, or more than 1 year.

In some cases, a prime-boost immunization schedule is followed. For example, in some cases, a first immunogenic composition (prime) includes nonstructural antigens as recombinant protein, DNA recombinant vector (viral, bacterial or fungal) and the second immunogenic composition (boost) includes Ad or rAd containing various HCV antigens (from different genotypes and subtypes) to induce cellular and humoral immune responses against multiple structural (core, F, E1, E2, p7) and non structural antigens (NS2, NS3, NS4, NS5 or NS3-NS5) of HCV and vice versa. The prime boost regimen can include polypeptide antigens along with Ad or rAd, with or without adjuvants. The prime boost regimen can include more than one serotype of Ad, chimeric, modified and recombinant Ad.

Thus, in some cases, a method of the present disclosure of inducing an immune response in an individual to one or more HCV antigens comprises: a) administering, at a first time, to the individual an HCV non-structural antigen; and b) administering, at a second time, to the individual an adenovirus, as described above. In some cases, a method of the present disclosure of inducing an immune response in an individual to one or more HCV antigens comprises: a) administering, at a first time, to the individual an adenovirus, as described above; b) administering, at a second time, to the individual an HCV non-structural antigen. The first time and the second time can be separated from one another by 1 hours to 1 year, e.g., from 1 hour to 12 hours, from 12 hours to 24 hours, from 2 days to 1 week, from 1 week to 1 month, from 1 month to 3 months, from 3 months to 6 months, or from 6 months to 1 year, or more than 1 year.

In some cases, a prime-boost immunization schedule is followed. For example, in some cases, a first immunogenic composition comprising Ad or rAd is administered at a first time; and second immunogenic composition, comprising a non-adenoviral vector encoding one or more HCV antigens, is administered at a second time. Examples of non-adenoviral vectors include but are not limited to adeno-associated virus, lentivirus, retroviruses, herpes virus, poxviruses, vesicular stomatitis virus, alpha virus, measles virus, plant viruses, alpha virus, insect virus, equine virus, papaya mosaic virus, cytomegalovoirus, vaccinia, modified vaccinia Ankara virus (MVA), polio virus, Marba virus etc.), bacterial vector vaccines (such as Salmonella, Shigella, E. coli, Lactococcus lactis, Listeria sp., Lactobacillus sp.), fungal vectors (such as heat killed recombinant Saccharomyces yeast), plant viruses, virus-like particles (VLPs), virosomes, DNA vector, synthetic vaccine particles, synthetic biomimetic supramolecular biovectors, depathogenized viral/bacterial strains (such as NIBRG14 from H5N1).

In some cases, a first immunogenic composition comprising a non-adenoviral vector encoding one or more HCV antigens is administered at a first time; and a second immunogenic composition comprising Ad or rAd is administered at a second time.

In some cases, a method of the present disclosure comprises administering adenovirus as an adenoviral virion. In some cases, a method of the present disclosure comprises administering adenovirus or adenoviral proteins in a dendritic cell. Thus, e.g., in some cases, a method of the present disclosure for inducing an immune response in an individual to one or more HCV antigens comprises: a) obtaining dendritic cells (DCs) from the individual; b) genetically modifying the DCs to express one or more adenoviral proteins; and c) administering the genetically modified DCs to the individual. In some cases, a method of the present disclosure for inducing an immune response in an individual to one or more HCV antigens comprises: a) obtaining DCs from the individual; b) infecting the DCs with replication competent adenovirus or replication-defective adenovirus; and c) administering the infected DCs to the individual. In some cases, a method of the present disclosure for inducing an immune response in an individual to one or more HCV antigens comprises: a) obtaining DCs from the individual; b) introducing one or more adenoviral proteins, or RNA encoding one or more adenoviral proteins, into the DCs, thereby generating adenoviral protein-expressing DCs; and c) administering the adenoviral protein-expressing DCs to the individual.

In some cases, adenoviral proteins are fused with carriers such as keyhole limpet hemocyanin (KLH), hepatitis B virus core, etc. In some cases, a method of the present disclosure for inducing an immune response in an individual to one or more HCV antigens comprises administering to the individual a composition comprising an adenoviral protein fused to a carrier, where suitable carriers include KLH, HBV core antigen, and the like.

As noted above, a method of the present disclosure comprises administering to an individual in need thereof an effective amount of an adenovirus composition. In some cases, an effective amount of an adenovirus composition is an amount that, when administered in one or more doses to an individual in need thereof, induces an immune response to more than one HCV antigen. In some cases, an effective amount of an adenovirus composition is an amount that, when administered in one or more doses to an individual in need thereof, induces an immune response to an HCV core antigen and at least one of HCV F, HCV NS3, HCV NS4, and HCV NS5. In some cases, an effective amount of an adenovirus composition is an amount that, when administered in one or more doses to an individual in need thereof, induces an immune response to an HCV NS3 antigen and at least one of HCV F, HCV core, HCV NS4, and HCV NS5. In some cases, an effective amount of an adenovirus composition is an amount that, when administered in one or more doses to an individual in need thereof, induces an immune response to HCV core antigen, HCV F, HCV NS3, HCV NS4, and HCV NS5. In some cases, an effective amount of an adenovirus composition is an amount that, when administered in one or more doses to an individual in need thereof, induces an immune response to HCV core antigen, HCV F, HCV NS3, and HCV NS4. In some cases, an effective amount of an adenovirus composition is an amount that, when administered in one or more doses to an individual in need thereof, induces an immune response to HCV core antigen, HCV NS3, HCV NS4, and HCV NS5. In some cases, the immune response is a humoral immune response. In some cases, the immune response is a cellular immune response.

In some cases, an effective amount of an adenovirus composition is an amount that, when administered in one or more doses to an individual in need thereof, induces neutralizing and/or non-neutralizing antibody to HCV in the individual. In some cases, an effective amount of an adenovirus composition is an amount that, when administered in one or more doses to an individual in need thereof, induces neutralizing antibody to HCV in the individual.

In some cases, an effective amount of an adenovirus composition is an amount that, when administered in one or more doses to an individual in need thereof, induces a cytotoxic T lymphocyte (CTL) response to HCV in the individual. In some cases, an effective amount of an adenovirus composition is an amount that, when administered in one or more doses to an individual in need thereof, induces a helper T lymphocyte (TH) response to HCV in the individual.

In some cases, an effective amount of an adenovirus composition is an amount that, when administered in one or more doses to an individual in need thereof, induces neutralizing and/or non-neutralizing antibody to HCV in the individual and induces a T helper and/or CTL response to HCV in the individual.

In some cases, an effective amount of an adenovirus composition is an amount that, when administered in one or more doses to an individual in need thereof, induces an effector immune response to HCV in the individual.

In some cases, a method of the present disclosure comprises multiple administration of an adenoviral nucleic acid or adenoviral polypeptide. For example, in some cases, a sequential immunization schedule is used, where a first composition comprising a first adenovirus nucleic acid from adenovirus of a first species or serotype is administered; and a second composition comprising a second adenovirus nucleic acid from adenovirus of a second species or serotype is administered from 1 day to 1 year (or more than 1 year) after the first composition is administered. For example, in some cases, a sequential immunization schedule is used, where a first composition comprising a first adenovirus nucleic acid from adenovirus of a first species or serotype is administered; and a second composition comprising a second adenovirus nucleic acid from adenovirus of a second species or serotype is administered from 1 day to 7 days, from 1 week to 2 weeks, from 2 weeks to 4 weeks, from 1 month to 6 months, or from 6 months to 1 year, or more than 1 year, after the first composition is administered.

### Adenoviral Proteins

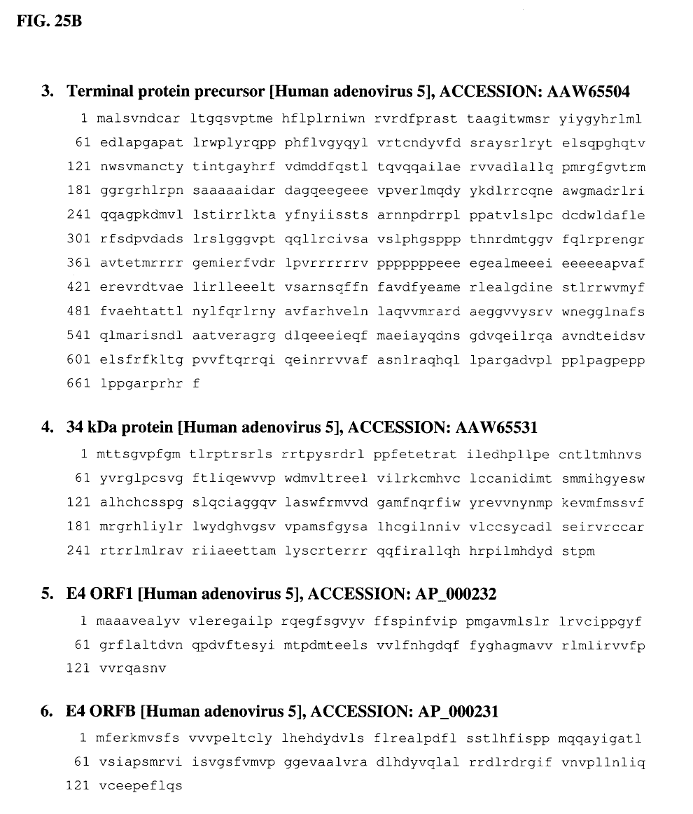

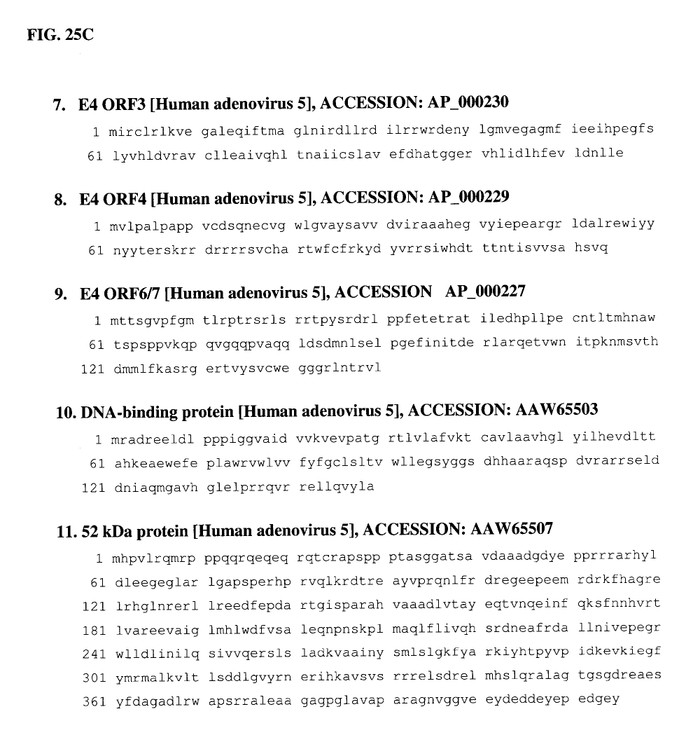

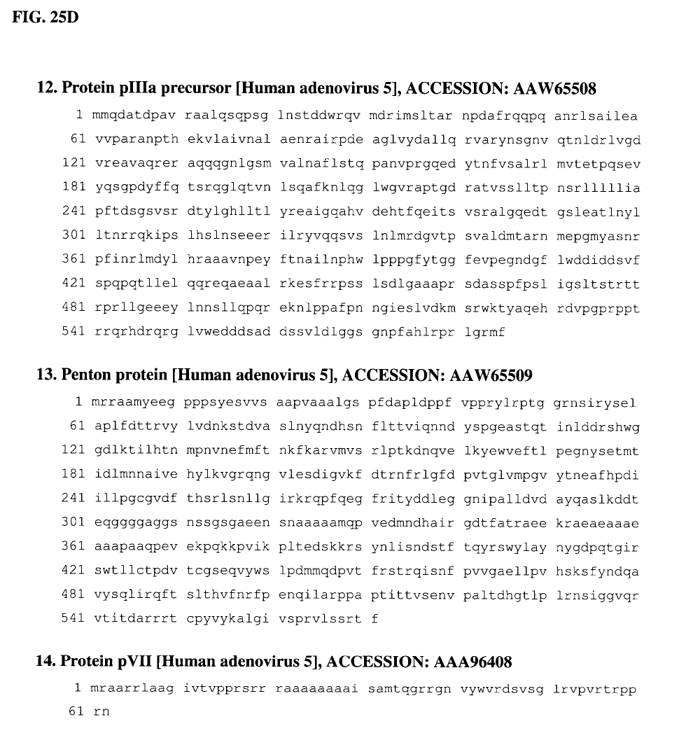

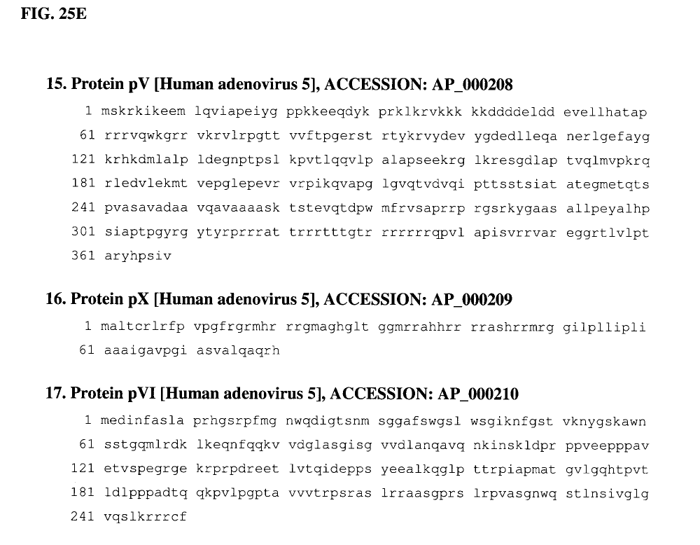

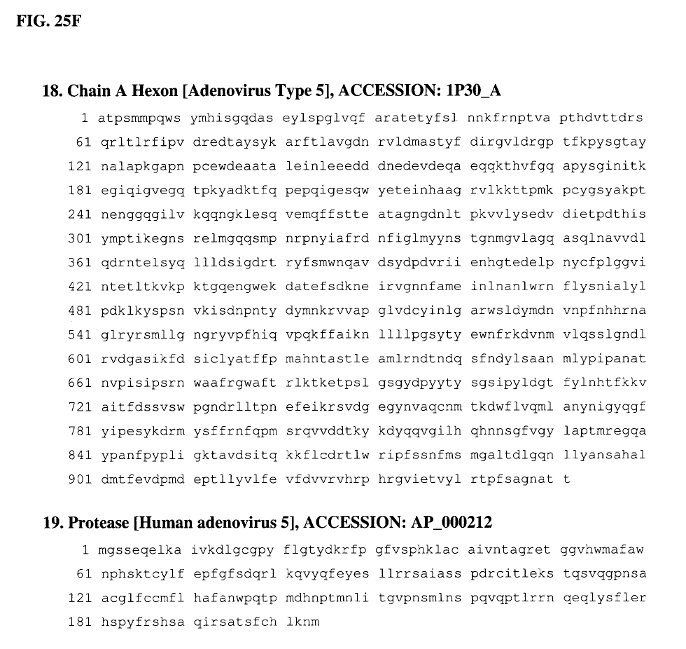

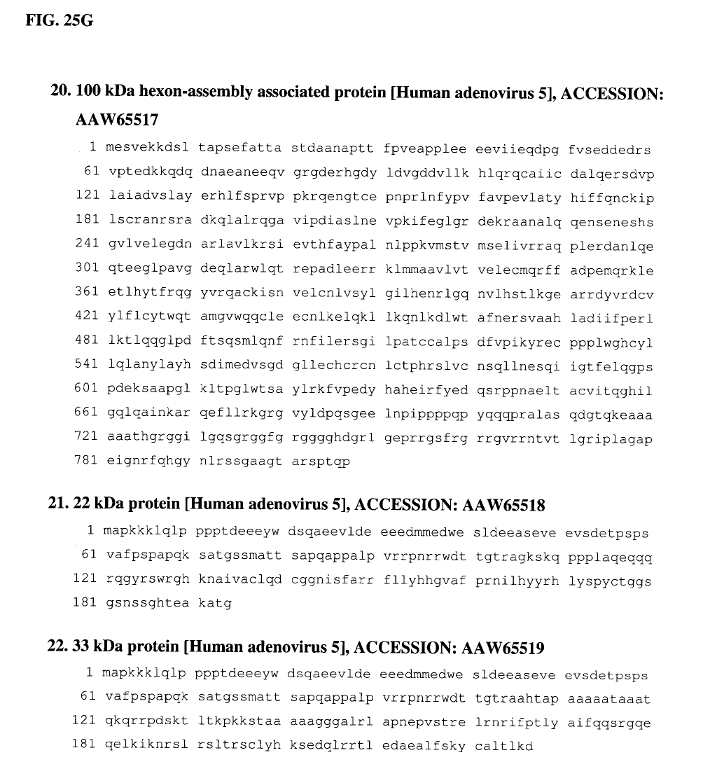

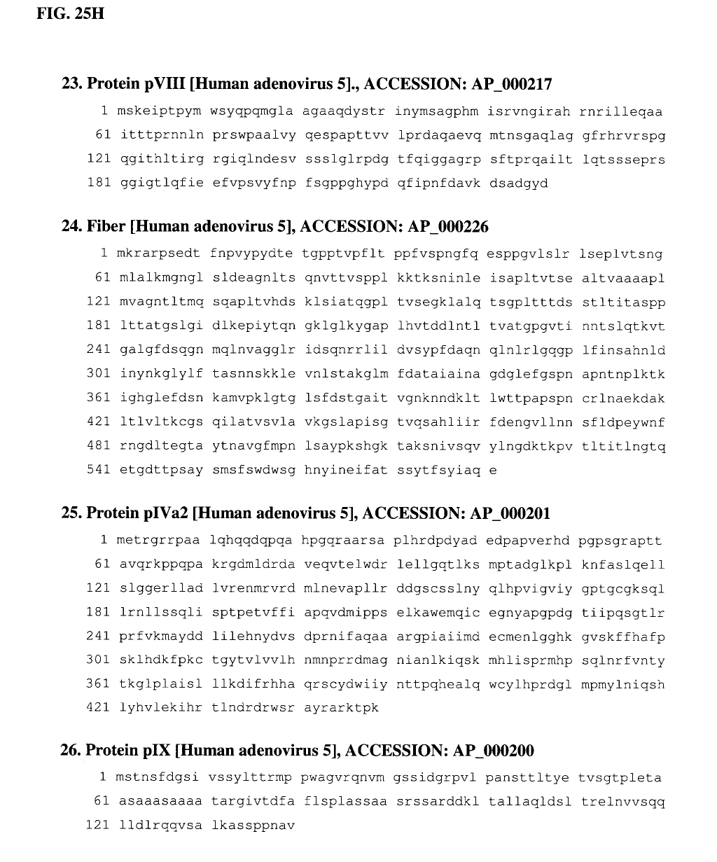

In some cases, an adenovirus composition comprises an adenovirus polypeptide, or a nucleic acid comprising a nucleotide sequence encoding an adenovirus polypeptide, where the adenovirus polypeptide(s) are depicted in FIGS. 25A-25I (Table 7). In some cases, the adenoviral polypeptides comprise amino acid sequences having at least 80%, at least 85%, at least 90%, at least 95%, at least 98%, at least 99%, or 100%, amino acid sequence identity to the amino acid sequence depicted in Table 7. In some cases, the adenovirus composition comprises all of the adenovirus polypeptides, or a nucleic acid comprising a nucleotide sequence encoding same, depicted in Table 7. In some cases, the adenovirus composition comprises 1, 2, 3, 4, 5, 6, 7, 8, 9, 10, 11, 12, 13, 14, 15, 16, 17, 18, 19, 20 21, 22, 23, 24, 25, or 26 of the adenovirus polypeptides, or a nucleic acid comprising a nucleotide sequence encoding same, depicted in Table 7. In some cases, the adenoviral polypeptides comprise amino acid sequences having at least 80%, at least 85%, at least 90%, at least 95%, at least 98%, at least 99%, or 100%, amino acid sequence identity to 1, 2, 3, 4, 5, 6, 7, 8, 9, 10, 11, 12, 13, 14, 15, 16, 17, 18, 19, 20 21, 22, 23, 24, 25, or 26 of the adenovirus polypeptides depicted in Table 7.

### Adjuvants

In some cases, an adenovirus composition is administered to an individual in need thereof, where the adenovirus composition comprises an adjuvant. Exemplary adjuvants include, but are not limited to: (1) oil-in-water emulsion formulations (with or without other specific immunostimulating agents such as muramyl peptides (see below) or bacterial cell wall components), such as for example (a) MF59™ (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12, IL-15, IL-28, etc.) (WO99/44636), etc.), interferons (e.g. gamma interferon), macrophage colony stimulating factor (M-CSF), tumor necrosis factor (TNF), colony-stimulating factors (e.g., GM-CSF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions e.g. EP-A-0835318, EP-A-0735898, EP-A-0761231; (7) oligonucleotides comprising CpG motifs (Krieg Vaccine 2000, 19, 618-622; WO 96/02555, WO 98/16247, WO 98/18810, WO 98/40100, WO 98/55495, WO 98/37919 and WO 98/52581), i.e., oligonucleotides containing at least one CG dinucleotide, where the cytosine is unmethylated; (8) a polyoxyethylene ether or a polyoxyethylene ester e.g. WO 99/52549; (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt e.g. WO 00/23105; (12) a saponin and an oil-in-water emulsion e.g. WO 99/11241; (13) a saponin (e.g. QS21)+3dMPL+IM2 (optionally including a sterol) e.g. WO 98/57659; (14) alphaGalCer and its derivatives; (16) toll-like receptor (TLR) agonists, NOD-like receptor (NLR) agonists, RIG-I agonists, agonists for C-type lectin receptors and other pathogen recognition receptor (PRR) agonists e.g., CpG ODNs, ISS-ODNs, rinatolimod, polyI:C and its derivatives, flagellin, ampligen, imidazoquinalines (e.g., imiquimod, resiquimod), muramyl dipeptides; (17) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. Adjuvants suitable for administration to a human included in some cases. In some cases, the adjuvant is aluminum hydroxide. In some cases, the adjuvant is aluminum potassium sulfate. In some cases, the adjuvant is aluminum phosphate. In some cases, the adjuvant is aluminum hydroxyphosphate sulfate. In some cases, the adjuvant is aluminum sulfate.

Further exemplary suitable adjuvants include, but are not limited to: cholera toxin B subunit, BCG, Pseudomonas aeruginosa exoprotein A, tocopherol, HBV core, E. coli heat labile toxins (such as LT-A, LT-B), Pertussis toxin, Diphtheria toxoid, tetanus toxoid, Aluminium salt-based adjuvants (such as Alum, Aluminum phosphate, Aluminum sulphate, Alhydrogel), Calcium phosphate, kaolin, monophosphoryl lipid A (MPL®) and its derivatives, glucopyranosyl lipid A, synthetic lipid A, Lipid A mimetics, Vitamin E, Depovax™, Saponins (Quil-A, AS01, AS02 (squalene+MPL+QS-21)), AS03, AS04 (alum+MPL®), Tomatin, Protolin, RC-529, Pluronic™, Monatides, Matrix-M, OM-174, Lipovac, IC-31, bacterial/mycobacterial peptides (such as KLK), polyphosphazene and its derivatives, Gellan, nucleotides (mono, di, poly), Gram⁺ and Gram⁻ non-pathogenic bacteria such as Caulobacter crescentus in live, inactivated and/or heat-killed form, etc.

An adenovirus composition can include one or more mucoadhesives such as sodium alginate, starch, lectins, thiolated polymers, GelVac™, sodium carboxymethylcellulose, hydroxylpropyl methylcellulose, carbomers, cetyl trimethyl ammonium bromide.

An adenovirus composition can include one or more additional adjuvant formulations such as oil-in-water emulsions, water-in-oil emulsions, nanoemulsions, particulate delivery systems, liposomes, microspheres, biodegradable microspheres, patches virosomes, proteoliposomes, proteasomes, Immunostimulatory complexes (ISCOMs, ISCOMATRIX), microparticles, nanoparticles, polymeric micro/nano particles, polymeric lamellar substrate particles (PLSP), microparticle resins, synthetic/biodegradable and biocompatible semisynthetic or natural polymers (such as PLG, PLGA, PLA, polycaprolactone, silicone polymer, polyesters, poly-dimethyl siloxane, sodium polystyrene sulphonate, polystyrene benzyl trimethyl ammonium chloride, polystyrene divinyl benzene resin, polyphosphazene, poly-[di-(carboxylactophenoxy)phosphazene] (PCPP), poly-(methylmethacrylate), dextran, polyvinylpyrrolidone, hyaluronic acid and derivatives, chitosan and its derivatives, polysaccharides, lipopolysaccharides, polycationic compound(s) (such as Poly-amino acids, poly-(γ-glutamic acid), poly-arginine-HCl, poly-L-lysine, polypeptides, biopolymers), cationic dimethyldioctadecyl ammonium (DDA), alpha-galactosyl ceramide and its derivatives, archaeal lipids and derivatives, lactanes, gallen, glycerolipids, cochleates, etc.

An adenovirus composition can include one or more additional adjuvant formulations such as oil-in-water emulsions or water-in-oil emulsions including edible oils (such as olive oil, mustard oil, vegetable oil, mineral oil etc.).

An adenovirus composition can include one or more additional surfactants and detergents (e.g., non-ionic detergents) (such as Tween-80, Polysorbate 80, Span 85, Stearyl tyrosine etc.).

In some cases, an adenovirus composition is administered to an individual in need thereof, where the adenovirus composition comprises one or more chemokines, one or more costimulatory molecules (e.g., CD40L, 4-BBL, anti-CD40 Mab), or one or more antibodies to coinhibitory molecules (e.g., anti-CTLA-4, anti-PD-1).

### Non-Adenovirus Polypeptides

As noted above, in some embodiments, an adenovirus used in a method of the present disclosure does not include a nucleotide sequence encoding a polypeptide other than an adenovirus polypeptide. In other instances, an adenovirus used in a method of the present disclosure includes a nucleotide sequence encoding a polypeptide other than an adenovirus polypeptide. In some cases, an adenovirus used in a method of the present disclosure includes a nucleotide sequence encoding a polypeptide other than an adenovirus polypeptide, where the non-adenovirus polypeptide is an HCV polypeptide. In some cases, an adenovirus used in a method of the present disclosure includes a nucleotide sequence encoding a polypeptide other than an adenovirus polypeptide, where the non-adenovirus polypeptide is from a pathogen other than HCV. In some cases, an adenovirus used in a method of the present disclosure includes a nucleotide sequence encoding a polypeptide such as an antigen from a pathogen (e.g., a pathogen other than HCV), or a tumor-associate antigen. The recombinant virus may comprise a plurality of antigen-encoding nucleotide sequences. The plurality of the antigen-encoding nucleotide sequences may be multiple copies of the same antigen-encoding sequence or multiple antigen sequences that differ from each other. The plurality of antigen-encoding nucleotide sequences may be derived from a single pathogen or cancer, different strains/serotypes of a pathogen or cancer or different kinds of pathogens or cancer. In some cases, the antigen sequences may include sequences one of which induces T cell responses against a pathogenic antigen(s) and/or another induces B cell (humoral) responses against another antigen.

In some cases, adenovirus including a nucleotide sequence encoding a polypeptide other than adenovirus polypeptide can be mixed together with another adenovirus or a non-adenoviral vector expressing different nucleotide sequence encoding for a second antigen.

Suitable antigens include, but are not limited to, an antigen derived from a pathogenic microorganism; a tumor-associated antigen; and the like. Antigens derived from a pathogenic microorganism include antigens derived from a virus, a bacterium, a fungus, a protozoan, or a helminth.

In some cases, an adenovirus composition is administered to an individual in need thereof, where the adenovirus composition comprises one or more inactivated/non-pathogenic/commensal gram positive or gram-negative bacteria, virus or their antigens.

Antigens Other than an Adenoviral Antigen

In some cases, an adenovirus used in a method of the present disclosure includes a nucleotide sequence encoding a polypeptide other than an adenovirus polypeptide, where the non-adenovirus antigen is a polypeptide, e.g., a full-length protein or a portion of an antigenic protein that contains an immunodominant antigen, a neutralizing antigen, or epitopes of a pathogenic antigen other than an HCV polypeptide. A suitable antigen can be any type of antigen known in the art. Antigens can be in variety of forms as described below.

A recombinant adenovirus vector can be constructed by one of ordinary skill in the art following procedures known in the literature. The amino acid sequence of an antigen encoded by the recombinant adenovirus vector may be natural, mutated, truncated, modified, optimized, or inactivated, and can from the same or different strains of the pathogen(s) or can be from the same cancer cell or multiple different cancer cells.

Antigens from Pathogenic Bacteria

In some cases, an adenovirus used in a method of the present disclosure includes a nucleotide sequence encoding an antigen derived from or associated with a pathogenic bacterium. In some cases, an adenovirus used in a method of the present disclosure includes a nucleotide sequence encoding one or more bacterial antigens, e.g., 1, 2, 3, 4, 5, or more bacterial antigens, from one or more bacteria.

Non-limiting examples of pathogenic bacteria include Mycobacteria, Streptococcus, Staphylococcus, Pseudomonas, Salmonella, Neisseria, and Listeria. In some cases, the bacteria is Neisseria gonorrhea, M. tuberculosis, M. leprae, Listeria monocytogenes, Streptococcus pneumoniae, S. pyogenes, S. agalactiae, S. viridans, S. faecalis, or S. bovis.

Other examples of bacteria contemplated include, but are not limited to, Gram positive bacteria (e.g., Listeria, Bacillus such as Bacillus anthracis, Erysipelothrix species), Gram negative bacteria (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species.

Additional non-limiting examples of specific infectious bacteria include Helicobacter pyloris, Borelia burgdorferi, Legionella pneumophila, Mycobacteria avium, M. intracellulare, M. kansaii, M. gordonae, M. africanum, Staphylococcus aureus, Neisseria meningitidis, Haemophilus influenzae, Bacillus anthracis, Corynebacterium diphtheriae, Erysipelothrix rhusiopathiae, Clostridium perfringens, Clostridium tetani, Enterobacter aerogenes, Klebsiella pneumoniae, Pasteurella multocida, Fusobacterium nucleatum, Streptobacillus moniliformis, Treponema pallidium, Treponema pertenue, Leptospira, Rickettsia, and Actinomyces israelli.

An antigen can be derived from any of the aforementioned bacteria.

Non-limiting examples of suitable bacterial antigens include pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and 85B and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

A bacterial antigen can be purified (e.g., at least 50% pure, at least 60% pure, at least 70% pure, at least 80% pure, at least 90% pure, at least 95% pure, at least 98% pure, or at least 99% pure, or more than 99% pure). A bacterial antigen can be an extract from a bacterial cell. A bacterial antigen can be synthetically produced, e.g., by recombinant means.

### Fungal Antigens

In some cases, an adenovirus used in a method of the present disclosure includes a nucleotide sequence encoding one or more fungal antigens, e.g., 1, 2, 3, 4, 5, or more fungal antigens, from one or more fungi.

Fungal antigens include, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidioides fungal antigens such as spherule antigens and other coccidioides fungal antigen components; and tinea fungal antigens such as trichophytin and other coccidioides fungal antigen components.

Fungal antigens can be obtained from Candida spp. including C. albicans, Aspergillus spp., Cryptococcus spp. including C. neoformans, Blastomyces sp., Pneumocytes spp., or Coccidioides spp.

### Parasite Antigens

In some cases, an adenovirus used in a method of the present disclosure includes a nucleotide sequence encoding a parasite antigen. Parasites include protozoan parasites and helminths. In some cases, an adenovirus used in a method of the present disclosure includes a nucleotide sequence encoding one or more parasitic antigens, e.g., 1, 2, 3, 4, 5, or more parasitic antigens, from one or more parasites.

Examples of parasites include Plasmodium spp., Toxoplasma gondii, Babesia spp., Trichinella spiralis, Entamoeba histolytica, Giardia lamblia, Enterocytozoon bieneusi, Naegleria, Acanthamoeba, Trypanosoma rhodesiense and Trypanosoma gambiense, Isospora spp., Cryptosporidium spp, Eimeria spp., Neospora spp., Sarcocystis spp., and Schistosoma spp.

Parasite antigens can be derived from Plasmodium spp. (such as RTS, S, TRAP, MSP-1, MSP-3, RAP1, RAP2 etc.), Toxoplasma spp. including T. gondii (such as SAG2, SAG3, Tg34), Entamoeba spp. including E. histolytica, Schistosoma spp., Trypanosoma cruzi Cryptosporidium spp., Angiostrongylus spp., Ancyclostoma spp., Wuchereria spp., Brugia spp., Giardia spp., Leishmania spp., Pneumonocystis spp., Enterobius spp., Ascaris spp., Trichuris spp., Trichomonas spp., Necator spp., Onchocerca spp., Dracanculus spp., Trichinella spp., Strongyloides spp., Opisthorchis spp., Paragonimus spp., Fasciola spp., or Taenia spp.

### Protozoan Antigens

In some cases, an adenovirus used in a method of the present disclosure includes a nucleotide sequence encoding a protozoan antigen. A protozoan antigen can be derived from any protozoan parasite, including, but not limited to, Giardia; a plasmodium species (e.g., Plasmodium falciparum); Toxoplasma gondii; a cryptosporidium; a Trichomonas species; a trypanosome (e.g., Trypanosoma cruzi); or Leishmania.

Protozoan antigens include, but are not limited to, e.g., plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmanial antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

### Helminth Antigens

In some cases, an adenovirus used in a method of the present disclosure includes a nucleotide sequence encoding a helminth antigen. Helminth antigens include antigens derived from flatworms, thorny-headed worms, and roundworms (nematodes).

### Viral Antigens

In some cases, an adenovirus used in a method of the present disclosure includes a nucleotide sequence encoding a one or more viral antigens, e.g., 1, 2, 3, 4, 5, or more viral antigens, from one or more viruses. In some cases, the viral antigen is an HCV antigen. In other embodiments, the antigen is other than an HCV antigen.

Viruses that can be the source of the viral antigen(s) include, but are not limited to, herpes viruses (HSV-1, HSV-2, VZV, EBV, CMV, HHV-6, HHV-8), influenza viruses (Flu A, B), hepatitis viruses (HepA, HepB, HepC, HepE), human immunodeficiency viruses (HIV-1, HIV-2), respiratory syncytial viruses, measles viruses, rhinoviruses, adenoviruses, SARS viruses, papillomaviruses, orthopoxviruses, West Nile viruses, and a dengue viruses. Viruses that can be the source of the viral antigen(s) include members of the Flaviviridae family of viruses. Viruses that can be the source of the viral antigen(s) include a flavivirus selected from the group consisting of dengue, Kunjin, Japanese encephalitis, West Nile, and yellow fever virus. Viruses that can be the source of the viral antigen(s) include lymphocytic choriomenignitis virus, hepatitis B virus, Epstein Barr virus, and human immunodeficiency virus. Viruses that can be the source of the viral antigen(s) include, but are not limited to: Retroviridae (e.g. human immunodeficiency viruses, such as HIV-1, also referred to as LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola-like viruses, Marburg viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arenaviridae (hemorrhagic fever viruses); Reoviridae (e.g. reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (e.g., adenoviruses); Herpesviridae (herpes simplex virus (HSV) 1 and 2), varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis, thought to be a defective satellite of hepatitis B virus), the agents of non-A, non-B hepatitis (class 1, internally transmitted; class 2, parenterally transmitted, i.e., Hepatitis C); Norwalk and related viruses, and astroviruses.

Suitable viral antigens include antigens from the herpesvirus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpesviruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefor; Baer et al., Nature (1984) 310:207-211, for the identification of protein coding sequences in an EBV genome; and Davison and Scott, J. Gen. Virol. (1986) 67:1759-1816, for a review of VZV.)

Suitable viral antigens include antigens from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Each of these proteins, as well as antigenic fragments thereof, will find use in the present composition and methods.

Suitable viral antigens include the 6-antigen from HDV (see, e.g., U.S. Pat. No. 5,378,814). Additionally, antigens derived from HBV, such as the core antigen, the surface antigen, sAg, as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, are suitable. See, e.g., “HBV Vaccines—from the laboratory to license: a case study” in Mackett, M. and Williamson, J. D., Human Vaccines and Vaccination, pp. 159-176, for a discussion of HBV structure; and U.S. Pat. Nos. 4,722,840, 5,098,704, 5,324,513; Beames et al., J. Virol. (1995) 69:6833-6838, Birnbaum et al., J. Virol. (1990) 64:3319-3330; and Zhou et al., J. Virol. (1991) 65:5457-5464.

Suitable viral antigens include antigens from members of filoviruses [e.g., Zaire, Sudan, Ivory Coast Ebola viruses, Marburg virus antigens such as structural proteins (membrane form of glycoproteins, soluble glycoproteins, NP, matrix proteins (VP24, VP40)) and nonstructural proteins (VP30, VP35)]. In some embodiments, the antigenic protein may be mutated so that it is less toxic to cells.

Suitable viral antigens include, but are not limited to, proteins from members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV-IIIb, HIV-SF2, HIV-LAV, HIV-LAI, HIV-MN); HIV-1-CM235, HIV-1-US4; HIV-2; simian immunodeficiency virus (SIV) among others. Additionally, antigens may also be derived from human papillomavirus (HPV) and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses.

Suitable viral antigens include the gp120 or gp140 envelope proteins from any of the above HIV isolates, including members of the various genetic subtypes of HIV, are known and reported (see, e.g., Myers et al., Los Alamos Database, Los Alamos National Laboratory, Los Alamos, N. Mex. (1992); Myers et al., Human Retroviruses and Aids, 1990, Los Alamos, N. Mex.: Los Alamos National Laboratory; and Modrow et al., J. Virol. (1987) 61:570-578, for a comparison of the envelope sequences of a variety of HIV isolates) and antigens derived from any of these isolates will find use in the present methods. Suitable viral antigens include proteins derived from any of the various HIV isolates, including any of the various envelope proteins such as gp160 and gp41, gag antigens such as p24gag and p55gag, as well as proteins derived from the pol and tat regions.

Suitable viral antigens include antigens of influenza virus. Specifically, the envelope glycoproteins HA and NA of influenza A can be used. Numerous HA subtypes of influenza A have been identified (Kawaoka et al., Virology (1990) 179:759-767; Webster et al., “Antigenic variation among type A influenza viruses,” p. 127-168. In: P. Palese and D. W. Kingsbury (ed.), Genetics of influenza viruses. Springer-Verlag, New York). Conserved antigens of influenza such as nucleoprotein, M2 and M1 can also be used in vaccine compositions. Thus, proteins derived from any of these isolates can also be used in the compositions and methods described herein.

### Cancer-Associated Antigens

In some cases, an adenovirus used in a method of the present disclosure includes a nucleotide sequence encoding a cancer-associated antigen. Cancer-associated antigens can be derived from the cell surface, cytoplasm, nucleus, organelles and the like of cells of tumor tissue. In some cases, an adenovirus used in a method of the present disclosure includes a nucleotide sequence encoding one or more cancer antigens, e.g., 1, 2, 3, 4, 5, or more cancer antigens, from one or more cancers.

Examples of cancer-associated antigens include, without limitation, antigens associated with hematological cancers such as leukemias and lymphomas, neurological tumors such as astrocytomas or glioblastomas, melanoma, breast cancer, lung cancer, head and neck cancer, gastrointestinal tumors such as gastric or colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

Cancer-associated antigens include, e.g., mutated oncogenes; viral proteins associated with tumors; and tumor mucins and glycolipids. The antigens may be viral proteins associated with tumors. Certain antigens may be characteristic of tumors (one subset being proteins not usually expressed by a tumor precursor cell), or may be a protein which is normally expressed in a tumor precursor cell, but having a mutation characteristic of a tumor. Other antigens include mutant variant(s) of the normal protein having an altered activity or subcellular distribution, e.g., mutations of genes giving rise to tumor antigens.

Specific non-limiting examples of suitable tumor antigens include: CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), Pmel 17 (gp100), GnT-V intron V sequence (N-acetylglucoaminyltransferase V intron V sequence), Prostate Ca psm, PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

Suitable cancer-associated antigens include, e.g., Melan-A/MART-1, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100.sup.Pmel117, PRAME, NY-ESO-1, brain glycogen phosphorylase, SSX-1, SSX-2 (HOM-MEL-40), SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, Smad family of tumor antigens, 1mp-1, EBV-encoded nuclear antigen (EBNA)-1, or c-erbB-2.

### Compositions Comprising an Adenovirus Nucleic Acid and a Non-Adenoviral Polypeptide

In some cases, an adenovirus composition for use in a method of the present disclosure comprises an adenovirus nucleic acid, as described above, and a non-adenoviral polypeptide. In some cases, the polypeptide is an antigen. For example, in some cases, the antigen is an antigen from a pathogen, a tumor-associated antigen, etc., as described above.

An antigen can be a whole cell extract, a cell lysates, a whole cell, a whole live cell, a whole inactivated cell, a whole irradiated cell, etc. Antigens may be crude, purified, or recombinant form. In some cases, an antigen is at least 50% pure, at least 60% pure, at least 70% pure, at least 80% pure, at least 90% pure, at least 95% pure, at least 98% pure, or at least 99% pure, or more than 99% pure.

An adenovirus composition can comprise a single type of antigen. An adenovirus composition can include 2 or more different antigens. Adenovirus composition can include 2, 3, 4, 5, 6, or more than 6, different antigens. Where an adenovirus composition includes more than one antigen, the more than one antigen can be from the same pathogenic organism, or from the same cancer cell. Where an adenovirus composition includes more than one antigen, the more than one antigen can be from two or more different pathogenic organisms, or from two or more different cancer cells or two or more different types of cancers.

An antigen can be in the form of a protein, a lipopolysaccharide, a lipoprotein, a proteoglycan, glycoproteins, glycosaminoglycans, a fragment of a protein (e.g., less than full-length protein), etc.

Suitable antigens include, e.g., peptides, modified peptides, conformationally-constrained synthetic peptides, lipopeptides, monolipopeptides, dilipopeptides, peptides conjugated or fused to proteins as antigens. See, e.g., U.S. Pat. No. 8,198,400. Suitable antigens include, e.g., proteins, purified or recombinant proteins, recombinant fusion proteins, proteins and peptides conjugated to toll-like receptor (TLR) agonists, glycoproteins, glycolipoproteins, polysaccharides, polysaccharide conjugates, lipids, glycolipids and carbohydrates.

An antigen or antigenic composition can be obtained from live viruses, dead viruses, attenuated viruses, bacteria, fungi, protozoa, helminths, etc.

In some cases, an adenovirus composition containing antigens other than an adenoviral antigen, or comprising an adenovirus encoding a polypeptide antigen other than an adenovirus polypeptide, can comprise one or more of an adjuvant, a surfactant, a detergent, and a mucoadhesive, where suitable adjuvants, surfactants, detergents, and mucoadhesives are described elsewhere herein.

In some cases, an adenovirus composition containing antigens other than an adenoviral antigen, or comprising an adenovirus encoding a polypeptide antigen other than an adenovirus polypeptide, can comprise an immunostimulatory or an immunomodulatory agent, where suitable immunostimulatory and immunomodulatory agents are described elsewhere herein. In some cases, an adenovirus encoding a polypeptide antigen other than an adenovirus polypeptide can comprise a nucleotide sequence encoding an immunostimulatory polypeptide or an immunomodulatory polypeptide, where suitable immunostimulatory and immunomodulatory polypeptides are described elsewhere herein.

In some cases, a mixture of different recombinant adenovirus vectors containing antigen and/or immunostimulatory sequences and/or immunomodulatory sequences can be mixed together for administration or administered at different sites simultaneously or sequentially.

In certain embodiments, an adenovirus composition containing antigens other than an adenoviral antigen, or comprising an adenovirus encoding a polypeptide antigen other than an adenovirus polypeptide, can be used in a prime-boost immunization regimen simultaneously or sequentially.

### Methods of Treating an HCV Infection

The present disclosure provides a method of treating an HCV infection in an individual, the method comprising inducing or enhancing an immune response to one or more HCV antigens in the individual. Methods of inducing an immune response to an HCV infection are those described above. The method generally involves administering to the individual an immunogenic adenovirus composition, where the adenovirus nucleic acid or adenovirus polypeptide present in the immunogenic adenovirus composition induces an immune response in the individual to one or more HCV antigens. In some cases, the adenovirus is a wild-type adenovirus. In some cases, the adenovirus is a recombinant adenovirus.

In some cases, an effective amount of an adenovirus composition is an amount that, when administered in one or more doses to an individual in need thereof, reduces HCV viral load in the individual. In some cases, an effective amount of an adenovirus composition is an amount that, when administered in one or more doses to an individual in need thereof, is effective to achieve a 1.5-log, a 2-log, a 2.5-log, a 3-log, a 3.5-log, a 4-log, a 4.5-log, or a 5-log reduction in viral titer in the serum of the individual.

In some cases, an effective amount of an adenovirus composition is an amount that, when administered in one or more doses to an individual in need thereof, is effective to reduce a serum level of HCV in the individual. For example, in some embodiments, an effective amount of an adenovirus composition is an amount that, when administered in one or more doses to an individual in need thereof, is effective to reduce the level of serum HCV in the individual to from about 1000 genome copies/mL serum to about 5000 genome copies/mL serum, to from about 500 genome copies/mL serum to about 1000 genome copies/mL serum, or to from about 100 genome copies/mL serum to about 500 genome copies/mL serum. In some embodiments, an effective amount of an adenovirus composition is an amount that, when administered in one or more doses to an individual in need thereof, is effective to reduce HCV viral load to lower than 100 genome copies/mL serum.

In some embodiments, an effective amount of an adenovirus composition is an amount that, when administered in one or more doses to an individual in need thereof, is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Gen-Probe Inc. (San Diego) and Chiron Corporation, and sold by Chiron Corporation under the trade name Procleix®, which NAT simultaneously tests for the presence of HIV-1 and HCV. See, e.g., Vargo et al. (2002) Transfusion 42:876-885.

As described above, in some cases, an adenovirus composition for use in a subject method does not include nucleotide sequences encoding non-adenovirus polypeptides.

As described above, in some cases, an adenovirus composition for use in a subject method includes nucleotide sequences encoding non-adenovirus polypeptides. As described above, in some cases, the non-adenovirus polypeptide is an antigen, e.g., an antigen associated with a pathogen, a cancer-associated antigen, etc. In some cases, an adenovirus composition containing antigens other than an adenoviral antigen, or comprising an adenovirus encoding a polypeptide antigen other than an adenovirus polypeptide, can comprise one or more of an adjuvant, a surfactant, a detergent, and a mucoadhesive, where suitable adjuvants, surfactants, detergents, and mucoadhesives are described elsewhere herein.

In some cases, an adenovirus composition containing antigens other than an adenoviral antigen, or comprising an adenovirus encoding a polypeptide antigen other than an adenovirus polypeptide, can comprise an immunostimulatory or an immunomodulatory agent, where suitable immunostimulatory and immunomodulatory agents are described elsewhere herein. In some cases, an adenovirus encoding a polypeptide antigen other than an adenovirus polypeptide can comprise a nucleotide sequence encoding an immunostimulatory polypeptide or an immunomodulatory polypeptide, where suitable immunostimulatory and immunomodulatory polypeptides are described elsewhere herein.

In some cases, a mixture of different recombinant adenovirus vectors containing antigen and/or immunostimulatory sequences and/or immunomodulatory sequences can be mixed together for administration or administered at different sites simultaneously or sequentially.

In certain embodiments, an adenovirus composition containing antigens other than an adenoviral antigen, or comprising an adenovirus encoding a polypeptide antigen other than an adenovirus polypeptide, can be used in a prime-boost immunization regimen simultaneously or sequentially.

### Combination Therapy

In some embodiments, a subject method of treating an HCV infection in an individual comprises: a) inducing an immune response in the individual to one or more HCV antigens, as described above; and b) administering at least one additional therapeutic agent that treats an HCV infection. In some embodiments, the at least one additional therapeutic agent is an antiviral agent, e.g., an agent that has activity in inhibiting HCV or an agent that has activity in inhibiting a coinfecting pathogen (e.g., an agent that has activity in inhibiting HIV, HBV etc.).

In some embodiments, the at least one additional therapeutic agent is an anticancer agent such as chemotherapeutic cytotoxic agents, oncolytic viruses, anticancer therapeutic antibodies and vaccines, which can treat hepatocellular carcinoma and/or other cancers along with inducing immune responses against HCV.

In some embodiments, the at least one additional therapeutic agent is a combination of ombitasvir, paritaprevir, and ritonavir. In some embodiments, the at least one additional therapeutic agent is Solvaldi. In some embodiments, the at least one additional therapeutic agent is Harvoni. In some embodiments, the at least one additional therapeutic agent is Olysio. In some embodiments, the at least one additional therapeutic agent is an agent described in U.S. Patent Publication No. 2014/0309189 or 2014/0309164.

In some embodiments, the at least one additional therapeutic agent is an HCV polymerase inhibitor, ribavirin, viramidine, clemizole, filibuvir (PF-00868554), HCV POL, NM 283 (valopicitabine), MK-0608, 7-Fluoro-MK-0608, MK-3281, IDX-375, ABT-072, ABT-333, ANA598, BI 207127, GS 9190, PSI-6130, R1626, PSI-6206, PSI-938, PSI-7851, sofosbuvir (Sovaldi, PSI-7977, GS-7977), RG1479, RG7128, HCV-796 VCH-759 or VCH-916.

Sovaldi has the structure:

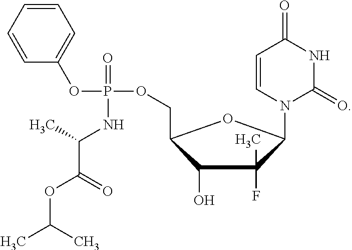

In some embodiments, the at least one additional therapeutic agent is a p38 MAPK inhibitor. Suitable p38 MAPK inhibitors include, e.g., SB203580 (4-(4-Fluorophenyl)-2-(4-methylsulfinylphenyl)-5-(4-pyridyl)-1H-imidazole).

In some embodiments, the at least one additional therapeutic agent includes interferon-alpha (IFN-α). Any known IFN-α can be used in a combination therapy. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods.

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Effective dosages of Infergen™ consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS™PEGylated IFN-α2a contain an amount of about 90 μg to 270 μg, or about 180 μg, of drug per dose. Effective dosages of PEG-INTRON™ PEGylated IFN-α2b contain an amount of about 0.5 μg to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 μg, or about 60 μg to about 180 μg, or about 90 μg to about 120 μg, of drug per dose. IFN-α can be administered daily, every other day, once a week, three times a week, every other week, three times per month, once monthly, substantially continuously or continuously.

In some embodiments, the at least one additional suitable therapeutic agent includes ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as the adenovirus. Of course, other types of administration are contemplated, such as by nasal spray, transdermally, by suppository, by sustained release dosage form, etc. Any suitable form of administration can be utilized so long as the proper dosages are delivered without destroying the active ingredient.

Ribavirin can be administered in an amount ranging from about 400 mg to about 1200 mg, from about 600 mg to about 1000 mg, or from about 700 to about 900 mg per day.

### Levovirin

In some embodiments, the at least one additional suitable therapeutic agent includes levovirin. Levovirin is the L-enantiomer of ribavirin. Levovirin is manufactured by ICN Pharmaceuticals.

Levovirin has the following structure:

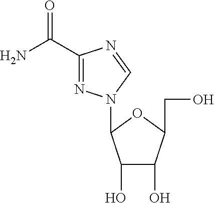 [SMILES: NC(=O)c1ncn(C2OC(CO)C(O)C2O)n1]

### Viramidine

In some embodiments, the at least one additional suitable therapeutic agent includes viramidine. Viramidine is a 3-carboxamidine derivative of ribavirin, and acts as a prodrug of ribavirin. It is efficiently converted to ribavirin by adenosine deaminases.

Viramidine has the following structure:

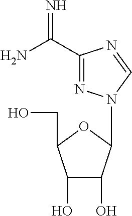 [SMILES: N=C(N)c1ncn(C2OC(CO)C(O)C2O)n1]

Nucleoside analogs that are suitable for use in a subject combination therapy include, but are not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguanine, 2-amino-α-L-ribofuranl[1′,2′:4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,2′:4,5]oxazoline, O²,O²-anhydro-β-L-arabinofuranosyluracil, 2′-deoxy-β-L-uridine, 3′5′-Di-O-benzoyl-2′deoxy-4-thio β-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula 1 of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both Merck/Isis); and the like.

### HCV NS3 Inhibitors

In some embodiments, the at least one additional suitable therapeutic agent includes an HCV NS3 inhibitor. Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH6 (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Patent Publication Nos. 2003019067, 20030187018, and 20030186895; and the like.

In some cases, the at least one additional therapeutic agent includes NS3 inhibitors that are specific NS3 inhibitors, e.g., NS3 inhibitors that inhibit NS3 serine protease activity and that do not show significant inhibitory activity against other serine proteases such as human leukocyte elastase, porcine pancreatic elastase, or bovine pancreatic chymotrypsin, or cysteine proteases such as human liver cathepsin B.

### NS5B Inhibitors

In some embodiments, the at least one additional suitable therapeutic agent includes an NSSB inhibitor. Suitable HCV non-structural protein-5 (NS5; RNA-dependent RNA polymerase) inhibitors include, but are not limited to, a compound as disclosed in U.S. Pat. No. 6,479,508 (Boehringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-2681; a benzothiadiazine compound as disclosed in Dhanak et al. (2002) J. Biol Chem. 277(41):38322-7; an NS5B inhibitor as disclosed in WO 02/100846 A1 or WO 02/100851 A2 (both Shire); an NS5B inhibitor as disclosed in WO 01/85172 A1 or WO 02/098424 A1 (both Glaxo SmithKline); an NS5B inhibitor as disclosed in WO 00/06529 or WO 02/06246 A1 (both Merck); an NS5B inhibitor as disclosed in WO 03/000254 (Japan Tobacco); an NS5B inhibitor as disclosed in EP 1 256,628 A2 (Agouron); JTK-002 (Japan Tobacco); JTK-109 (Japan Tobacco); and the like.

Of particular interest in many embodiments are NS5 inhibitors that are specific NS5 inhibitors, e.g., NS5 inhibitors that inhibit NS5 RNA-dependent RNA polymerase and that lack significant inhibitory effects toward other RNA dependent RNA polymerases and toward DNA dependent RNA polymerases.

In some embodiments, the at least one additional therapeutic agent is a peptide, peptide mimetic or modified peptide, which inhibits the interaction of HCV proteins with host receptors.

In some cases, a method of the present disclosure of inducing immune responses against HCV antigens comprises administering an adenoviral composition to an individual in need thereof, and further comprising administering to the individual an effective amount of at least one additional therapeutic agent, e.g., a monoclonal antibody directed against negative receptors such as PD1 and CTLA-4; antibody directed against co-stimulatory receptors e.g., CD134 and CD137; CDP-860 (anti-CD18), antibody directed against cytokines such as IL-10 and TGF-b, antibody directed against apoptotic cells e.g., anti-phosphatidylserine Mab and the like.

In some cases, an adenovirus can be engineered to express siRNA, shRNA or antisense DNA against a pathogen to inhibit the replication of pathogens such as HIV, HBV, HCV etc., along with providing immunotherapy to induce HCV specific immune responses.

In some embodiments, the at least one additional therapeutic agent is an antiviral agent e.g., an agent that has activity in inhibiting HIV, HBV influenza, etc. In some embodiments, the at least one additional therapeutic agent is an anticancer agent.

In some embodiments, the methods of present disclosure can be used to induce immune responses in humans. In some embodiments, the methods of present disclosure can be used to induce immune responses in non-human mammals, such as mouse, rat, donkey, rabbit, monkey, dogs, or cats. Delivery to non-human mammals can be for therapeutic purposes. Delivery to non-human mammals can be for use in an experimental context, for instance examining the mechanisms of inducing HCV specific immune responses and modulation of immune responses etc.

In some embodiments, the methods of present disclosure can be used to enhance and/or modify the therapeutic and protective effects and/or reduce frequency and dosing of therapeutic agent and/or vaccine.

### Formulations, Dosages, and Routes of Administration

An adenovirus composition to be administered according to a method of the present disclosure can include one or more pharmaceutically acceptable excipients; and can be formulated in any of a variety of ways, that may depend, e.g., on the route of administration. Pharmaceutically acceptable excipients are known to those skilled in the art, and have been amply described in a variety of publications, including, for example, A. Gennaro (1995) “Remington: The Science and Practice of Pharmacy”, 19th edition, Lippincott, Williams, & Wilkins. Suitable excipient vehicles include, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins.

An adenovirus composition to be administered according to a method of the present disclosure can be incorporated into a variety of formulations for administration. More particularly, an adenovirus composition can be formulated into pharmaceutical compositions by combination with appropriate, pharmaceutically acceptable carriers or diluents, and may be formulated into preparations in solid, semi-solid, freeze-dried, liquid or gaseous forms, such as tablets, capsules, powders, granules, ointments, solutions, suppositories, injections, skin patches, inhalants and aerosols.

In pharmaceutical dosage forms, an adenovirus composition may be administered alone or in appropriate association, as well as in combination, with other pharmaceutically active compounds. An adenovirus composition, an antigen, adjuvant and/or therapeutic drug can be administered concurrently, simultaneously, sequentially or at different times and via different routes. The following methods and excipients are merely exemplary and are in no way limiting.

For oral preparations, an adenovirus composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An adenovirus composition can be formulated into preparations for injection by dissolving, suspending or emulsifying the composition in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An adenovirus composition can be utilized in aerosol formulation to be administered via inhalation. An adenovirus composition to be administered according to a method of the present disclosure can be formulated into pressurized acceptable propellants such as dichlorodifluoromethane, propane, nitrogen and the like.

Furthermore, an adenovirus composition can be made into suppositories by mixing with a variety of bases such as emulsifying bases or water-soluble bases. An adenovirus composition can be administered rectally via a suppository. The suppository can include vehicles such as cocoa butter, carbowaxes and polyethylene glycols, which melt at body temperature, yet are solidified at room temperature.

An adenovirus composition to be administered according to a method of the present disclosure can also be administered in the form of liposomes. Liposomes can be given by a variety of routes, oral, nasal, parenteral, trans-dermal, inhalation etc. As is known in the art, liposomes are derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. An adenovirus composition in liposome form can contain, in addition to an adenovirus composition, one or more of a stabilizer, a preservative, an excipients, and the like. Exemplary lipids are the phospholipids and the phosphatidylcholines (lecithins), both natural and synthetic. Methods to form liposomes are known in the art. for example, Prescott, Ed., Methods in Cell Biology, Volume XIV, Academic Press, New York, N.Y. (1976), p. 33 et seq.

An adenovirus composition to be administered according to a method of the present disclosure can also be administered in the form of microspheres, nanoparticles etc.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise an adenovirus composition as a solution in sterile water, normal saline or another pharmaceutically acceptable carrier.

An adenovirus composition to be administered according to a method of the present disclosure can be formulated for topical administration. Topical administration includes administration to the skin or mucosa, including surfaces of the lung eye, nose, and ear. Suitable topical preparations include, e.g., skin patch preparation, transdermal patch preparation, cream, lotion, gel preparations, powder, ointment, paste, intranasal drops or gels.

Ointments are semi-solid preparations, which are typically based on petrolatum or other petroleum derivatives. Suitable ointments include oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (WIO) emulsions or oil-in-water (OIW) emulsions, and include, for example, cetyl alcohol, glyceryl monostearate, lanolin and stearic acid. Exemplary water-soluble ointment bases are prepared from polyethylene glycols of varying molecular weight.

Lotions are preparations to be applied to the skin surface without friction, and are typically liquid or semi liquid preparations in which solid particles, including the active agent, are present in a water or alcohol base. Lotions are usually suspensions of solids, and preferably, for the present purpose, comprise a liquid oily emulsion of the oil-in-water type. Lotions can be used for treating large body areas, because of the ease of applying a more fluid composition. Lotions may contain suspending agents to produce better dispersions as well as compounds useful for localizing and holding the active agent in contact with the skin, e.g., methyl cellulose, sodium carboxymethyl-cellulose, or the like. An example of a lotion formulation for use in conjunction with the present invention contains propylene glycol mixed with a hydrophilic petrolatum such as that which may be obtained under the trademark Aquaphor® from Beiersdorf, Inc. (Norwalk, Coon.).

Suitable creams can be viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil so phase in volume, and generally contains a humectant. The emulsifier in a cream formulation, as explained in Remington, supra, is generally a nonionic, anionic, cationic or amphoteric surfactant.

Gels formulations can be used. Gels are semisolid, suspension-/type systems. Single-phase gels contain organic macromolecules distributed substantially uniformly throughout the carrier liquid, which can be aqueous, but may also contain an alcohol and, optionally, an oil.

A topical formulation may also be delivered to the skin using conventional “transdermal”-type patches, wherein the agent (adenovirus composition) is contained within a laminated structure that serves as a delivery device to be affixed to the skin. In such a structure, the adenovirus composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. The particular polymeric adhesive selected will depend on the particular adenovirus composition, vehicle, etc., i.e., the adhesive must be compatible with all components of the drug-containing composition. In an alternative embodiment, the adenovirus composition-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

The term “unit dosage form,” as used herein, refers to physically discrete units suitable as unitary dosages for human and animal subjects, each unit containing a predetermined quantity of an active agent (e.g., adenovirus composition) calculated in an amount sufficient to produce the desired effect in association with a pharmaceutically acceptable diluent, carrier or vehicle. The specifications for the active agents depend on the particular compound employed and the effect to be achieved, and the pharmacodynamics associated with each compound in the host.

Other modes of administration will also find use. For instance, an adenovirus composition can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), or about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An adenovirus composition to be administered according to a method of the present disclosure can be administered as an injectable formulation. For example, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of an active agent (e.g., HKCC; antigen; etc.) adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Oral Formulations

In some embodiments, an adenovirus composition is formulated for oral delivery to an individual in need of such a composition.

For oral delivery, a formulation comprising an adenovirus composition will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

Suitable oral formulations also include an adenovirus composition, formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B.V.).

Suitable oral formulations also include an adenovirus composition formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

Suitable oral formulations also include an adenovirus composition, formulated as a food supplement (e.g. nutraceuticals, yogurt, bars) etc.

### Controlled Release Formulations

In some embodiments, an adenovirus composition is formulated in a controlled release formulation.

Controlled release can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of active agents in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher than normally encountered in the stomach.

One exemplary type of oral controlled release structure is enteric coating of a solid or liquid dosage form. The enteric coatings are designed to disintegrate in intestinal fluid for ready absorption. Delay of absorption of the active agent that is incorporated into a formulation with an enteric coating is dependent on the rate of transfer through the gastrointestinal tract, and so the rate of gastric emptying is an important factor. Some investigators have reported that a multiple-unit type dosage form, such as granules, may be superior to a single-unit type.

Suitable enteric coating agents include, but are not limited to, hydroxypropylmethylcellulose phthalate, methacrylic acid-methacrylic acid ester copolymer, polyvinyl acetate-phthalate and cellulose acetate phthalate.

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method.

Examples of carriers useful in solid dispersions include, but are not limited to, water-soluble polymers such as polyethylene glycol, polyvinylpyrrolidone, and hydroxypropylmethyl cellulose. Alternative carriers include phosphatidylcholine. Phosphatidylcholine is an amphoteric but water-insoluble lipid, which may improve the solubility of otherwise insoluble active agents in an amorphous state in phosphatidylcholine solid dispersions.

Other carriers include polyoxyethylene hydrogenated castor oil. An adenovirus composition can be included in a solid dispersion system with an enteric polymer such as hydroxypropylmethylcellulose phthalate and carboxymethylethylcellulose, and a non-enteric polymer, hydroxypropylmethylcellulose. Another solid dispersion dosage form includes incorporation of the drug of interest (e.g., an active agent) with ethyl cellulose and stearic acid in different ratios.

There are various methods commonly known for preparing solid dispersions. These include, but are not limited to, the melting method, the solvent method and the melting-solvent method.

Injectable microspheres are another controlled release dosage form. Injectable micro spheres may be prepared by non-aqueous phase separation techniques, and spray-drying techniques. Microspheres may be prepared using polylactic acid or copoly(lactic/glycolic acid).

Other controlled release technologies that may be used include, but are not limited to, SODAS (Spheroidal Oral Drug Absorption System), INDAS (Insoluble Drug Absorption System), IPDAS (Intestinal Protective Drug Absorption System), MODAS (Multiporous Oral Drug Absorption System), EFVAS (Effervescent Drug Absorption System), PRODAS (Programmable Oral Drug Absorption System), and DUREDAS (Dual Release Drug Absorption System) available from Elan Pharmaceutical Technologies. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

An adenovirus composition can be incorporated into any one of the aforementioned controlled released dosage forms, or other conventional dosage forms. The amount of active agent contained in each dose can be adjusted, to meet the needs of the individual patient, and the indication. One of skill in the art and reading this disclosure will readily recognize how to adjust the level of an active agent and the release rates in a controlled release formulation, in order to optimize delivery of an active agent and its bioavailability.

### Inhalational Formulations

An adenovirus composition to be administered according to a method of the present disclosure will in some embodiments be administered to a patient by means of a pharmaceutical delivery system for the inhalation route. The adenovirus composition may be formulated in a form suitable for administration by inhalation. The inhalational route of administration provides the advantage that the inhaled drug can bypass the blood-brain barrier. The pharmaceutical delivery system is one that is suitable for respiratory therapy by delivery of an active agent to mucosal linings of the bronchi. A system that depends on the power of a compressed gas to expel the adenovirus composition from a container can also be used. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed, the aerosol contains the therapeutically active compound (e.g., active agent), which can be dissolved, suspended, or emulsified in a mixture of a fluid carrier and a propellant. The aerosol can be in the form of a solution, suspension, emulsion, powder, or semi-solid preparation. Aerosols can be used for administration as fine, solid particles or as liquid mists via the respiratory tract of a patient. Various types of propellants known to one of skill in the art can be utilized. Suitable propellants include, but are not limited to, hydrocarbons or other suitable gas. In the case of the pressurized aerosol, the dosage unit may be determined by providing a value to deliver a metered amount.

An adenovirus composition can also be formulated for delivery with a nebulizer, which is an instrument that generates very fine liquid particles of substantially uniform size in a gas. For example, a liquid containing the adenovirus composition is dispersed as droplets. The small droplets can be carried by a current of air through an outlet tube of the nebulizer. The resulting mist penetrates into the respiratory tract of the patient.

There are several different types of inhalation methodologies which can be employed in connection with an adenovirus composition to be administered according to a method of the present disclosure. An adenovirus composition can be formulated with low boiling point propellants. Such formulations are generally administered by conventional meter dose inhalers (MDI's). Alternatively, an adenovirus composition can be formulated in aqueous or ethanolic solutions and delivered by conventional nebulizers. In some embodiments, such solution formulations are aerosolized using devices and systems such as disclosed within U.S. Pat. Nos. 5,497,763; 5,544,646; 5,718,222; and 5,660,166. An adenovirus composition can be formulated into dry powder formulations. Such formulations can be administered by simply inhaling the dry powder formulation after creating an aerosol mist of the powder. Technology for carrying such out is described within U.S. Pat. No. 5,775,320 issued Jul. 7, 1998 and U.S. Pat. No. 5,740,794 issued Apr. 21, 1998.

An adenovirus composition to be administered according to a method of the present disclosure will in some embodiments be formulated for vaginal delivery. An adenovirus composition for intravaginal administration can be formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An adenovirus composition will in some embodiments be formulated for rectal delivery. A formulation for intrarectal administration comprises an adenovirus composition formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel.

An adenovirus composition can include one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an adenovirus composition may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

### Dosages

The dosage of an adenovirus composition to be administered according to a method of the present disclosure can vary, depending on factors such as the clinical goals to be achieved, the age of the individual being treated, the physical status of the individual being treated, etc.

An adenovirus composition to be administered according to a method of the present disclosure can comprise adenovirus in an amount of from about 10³ genome copies per unit dosage form to about 10²⁰ genome copies per unit dosage form. For example, an adenovirus composition can comprise adenovirus in an amount of from about 10³ genome copies per unit dosage form to about 10⁴ genome copies per unit dosage form, from about 10⁴ genome copies per unit dosage form to about 10⁵ genome copies per unit dosage form, from about 10⁵ genome copies per unit dosage form to about 10⁶ genome copies per unit dosage form, from about 10⁶ genome copies per unit dosage form to about 10⁷ genome copies per ml, from about 10⁸ genome copies per unit dosage form to about 10⁹ genome copies per unit dosage form, from about 10⁹ genome copies per ml to about 10¹⁰ genome copies per unit dosage form, from about 10¹⁵ genome copies per unit dosage form to about 10²⁰ genome copies per unit dosage form, or more than 10²⁰ genome copies per unit dosage form.

For example, an adenovirus composition can comprise adenovirus in an amount of from about 10³ genome copies per ml to about 10²⁰ genome copies per ml. For example, an adenovirus composition can comprise genome copies in an amount of from about 10³ genome copies per ml to about 10⁴ genome copies per ml, from about 10⁴ genome copies per ml to about 10⁵ genome copies per ml, from about 10⁵ genome copies per ml to about 10⁶ genome copies per ml, from about 10⁶ genome copies per ml to about 10⁷ genome copies per ml, from about 10⁸ genome copies per ml to about 10⁹ genome copies per ml, from about 10⁹ genome copies per ml to about 10¹⁰ genome copies per ml, from about 10¹⁵ genome copies per ml to about 10²⁰ genome copies per ml, or more than 10²⁰ genome copies per ml.

In some embodiments, multiple doses of an adenovirus composition are administered. The frequency of administration of an adenovirus composition can vary depending on any of a variety of factors, e.g., severity of the symptoms, etc. For example, in some embodiments, an adenovirus composition is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an adenovirus composition, e.g., the period of time over which an adenovirus composition is administered, can vary, depending on any of a variety of factors, e.g., patient response, etc. For example, an adenovirus composition can be administered over a period of time ranging from about one hour to one day, from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, an adenovirus composition as described herein is administered with at least one additional agent (e.g., an additional immunogen, a therapeutic agent, an adjuvant etc.) as a single dose or in multiple doses, simultaneously or sequentially. Where an adenovirus composition and at least one additional agent are administered in multiple doses, the adenovirus composition and the at least one additional agent can be administered 1 minute apart, 1 day apart, 1 week apart, 2 weeks apart, 4 weeks apart, 6 weeks apart, 8 weeks apart, 10 weeks apart, 12 weeks apart, or more than 12 weeks apart.

### Routes of Administration

An adenovirus composition is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, intranodal, percutaneous, transdermal, intratumoral, topical application, intravenous, rectal, nasal, oral and other enteral and parenteral routes of administration. Routes of administration may be combined, if desired, or adjusted depending upon the agent and/or the desired effect. The composition can be administered in a single dose or in multiple doses.

An adenovirus composition can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intracranial, and intravenous routes, i.e., any route of administration other than through the alimentary canal. Parenteral administration can be carried to effect systemic or local delivery of the adenovirus composition. Where systemic delivery is desired, administration typically involves invasive or systemically absorbed topical or mucosal administration of pharmaceutical preparations.

An adenovirus composition can also be delivered to the subject by enteral administration. Enteral routes of administration include, but are not necessarily limited to, oral and rectal (e.g., using a suppository) delivery.

An adenovirus composition can also be delivered to the subject via a mucosal route of delivery. Mucosal routes of delivery include nasal, buccal, sublingual, vaginal, ocular, and rectal routes of administration.

In certain embodiments, an adenovirus composition is administered to a subject via a combination of different routes in the order indicated below:

i. systemic, mucosal;

ii. systemic, systemic, mucosal, mucosal;

iii. systemic, mucosal, systemic;

iv. mucosal, mucosal, systemic, systemic;

v. mucosal, systemic, systemic;

vi. mucosal, systemic, mucosal, for example.

When an adenovirus composition is administered systemically or mucosally more than once, the two or more systemic or mucosal administrations may be by the same systemic (for example, two intramuscular injections) or mucosal route (two IN/SL administrations) or different (for example, one intramuscular injection and one intravenous injection; one IN administration and one SL administration).

An adenovirus composition is administered to an individual using any available method, delivery or device such as vaccine patches, needles, microneedles, drop, syrup, tablets, capsules, pipette, dose-spray pumps, nasal dropper, inhalation devices, liquid or dry powder, freeze-dried powder, suspensions or solutions, spray devices, Accuspray™, thermoresponsive gels, jet injectors, Biojector™, Nasovak™, Bespak™, ointment, lotions, suppositories, gels etc.

In some embodiments, an adenovirus composition of the present disclosure may, if desired, be presented in a kit, pack or dispenser which may contain one or more unit dose forms containing the active ingredient. The kit may contain an adjuvant, a device for delivering the vaccine to a host.

### Subjects Suitable for Treatment

### Methods of Inducing an Immune Response to One or More HCV Antigens

Individuals who are suitable for treatment with method of inducing an immune response to one or more HCV antigens include individuals who are not infected with HCV. For example, a method of the present disclosure of inducing an immune response can be utilized as a prophylactic vaccine to induce immunity in an individual against HCV, such that upon subsequent exposure to HCV, induced preexisting immunity would reduce the likelihood that an HCV infection would be established in the individual. Individuals that are suitable for treatment with method of inducing an immune response to one or more HCV antigens include individuals who are at greater risk than the general population of becoming infected with HCV, where such individuals include intravenous drug users, medical personnel who come into contact with HCV-infected individuals, and the like. Individuals who are suitable for treatment with method of inducing an immune response to one or more HCV antigens include prospective liver transplant recipients.

### Methods of Treating an HCV Infection

Individuals that are suitable for treatment with method of treating an HCV infection of the present disclosure include individuals who have been diagnosed with an HCV infection. Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

In some cases, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Individuals that are suitable for treatment with a subject method for treating an HCV infection include individuals who have an HCV infection and, as a result of the HCV infection, suffer from liver fibrosis or hepatocellular carcinoma. Such individuals include HCV-infected individuals as described above.

Individuals that are suitable for treatment with a subject method for treating an HCV infection include individuals who have an HCV infection and are also co-infected with other pathogens e.g., HIV, HBV etc., or have a cancer. Such individuals include individuals infected with any of a variety of HCV genotypes, subtypes, or quasispecies, as described above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

The following materials and methods were used in the Examples provided below.

Sequence Alignment.

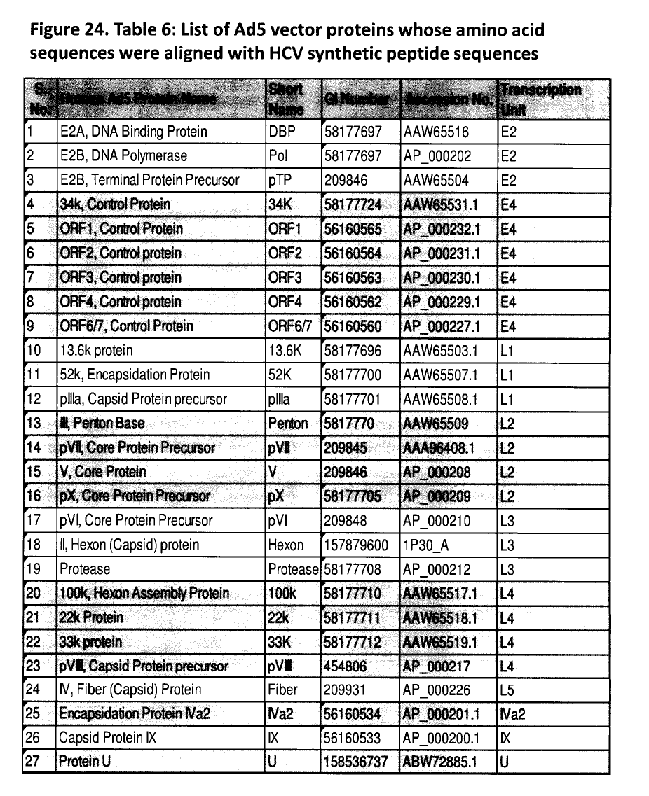

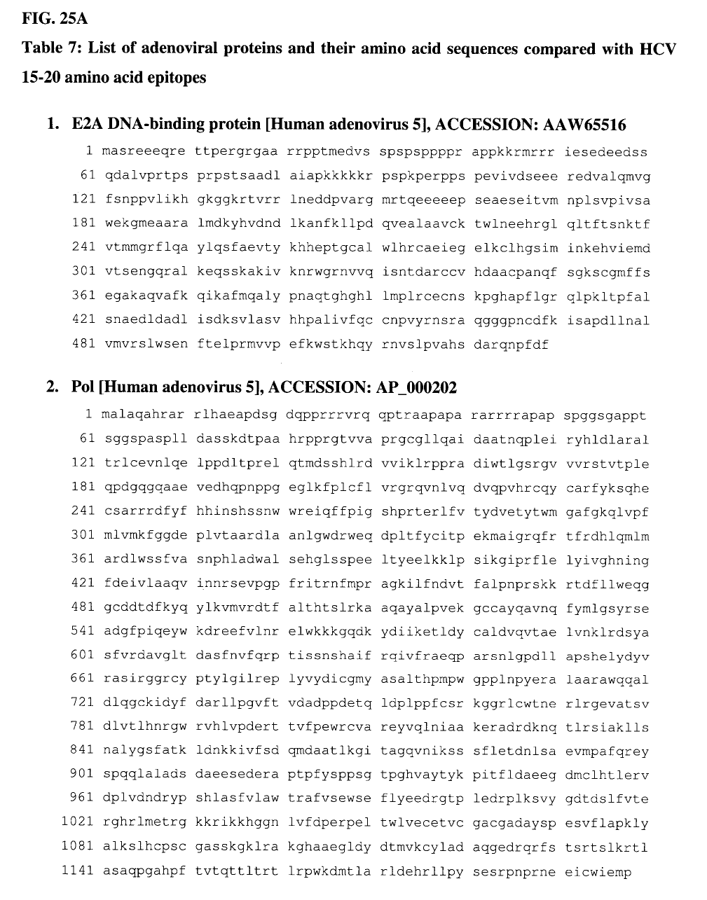

Human adenovirus 5 (adenoviral vector, Ad) protein sequences (Table 6; FIG. 24) were compared with 15-20 amino acid long peptide sequences from various HCV antigens (Core, F, NS3, NS4 and NS5) by sequence alignment using ClustalW software. Fifteen amino acid long peptides from F, core and NS3 and 20 amino acid long peptides from NS4 and NS5 were used. Sequence homology was documented as pairwise similarity or homology score and a heat map was prepared by Microsoft excel to present the distribution of each HCV antigen peptide homology across the different adenovirus proteins. Pairwise scores are the number of identities between the two sequences, divided by the length of the alignment, and calculated as a percentage, so a score of 25 means 25% homology, 30 means 30% homology and so on, in the aligned region. Number of adenovirus proteins showing various levels of homology (>25, >30, >35-40 and >50) is depicted in the tables 1-5.

Adenovirus (Ad) Vector.

Replication incompetent human adenovirus 5 with no transgene insert was amplified and titrated in human embryonic cell line 293A (HEK-293A) transformed with adenovirus E1 gene (QBiogene Inc., CA, USA) to provide complementarity for virus production. Recombinant adenoviruses (rAd), which express HCV antigens Core (rAd-core), F (rAd-F), NS3 (rAd-NS3), NS4 (rAd-NS4) or NS5 (rAd-NS5) have been prepared and reported earlier by us previously.

DNA Purification and PCR Amplification.

DNA was purified from Ad, rAd-NS5A, rAd-NS5B vector stocks. Briefly, 1×10⁹ pfu of each vector was taken in individual tubes and DNA was prepared by using High-Pure Viral Nucleic Acid Kit® (Roche Applied Bio). PCR reaction was set up with 10 μl template DNA obtained from the above preparation using 50 ul total reaction volume consisting of 1×PCR buffer, 10 μM dNTP, 25 μM of each primer (forward and reverse) and 1.25 unit of Taq polymerase. PCR tubes containing reaction mixtures was incubated in thermo-cycler with initial denaturation at 95° C. and 35 amplification cycles (95° C.: 30 Sec, 52° C.: 30 Sec, 68° C.: 60 Sec). PCR amplification products were run on 1% agarose gel at 80 volt to resolve amplification product along with 1 KB size Quick Load DNA Ladder (NEB Biolab, Germany).

Adjuvants.

Toll-like receptor agonists poly I:C (TLR 3 agonist) and resiquimod (TLR 7/8 agonist) were used as adjuvants with Ad vector for immunization.

Mice Immunizations.

Six to seven weeks old female C57Bl/6 mice were purchased from Charles River Laboratory (Charles River, Canada) and immunized once or twice using various doses (0.5×10⁶-2×10⁷ PFU/mouse) of Ad, rAd-HCVNS3, rAd-MtbAg85B, rAd-HIVnef, or a pool of HCV derived NS3 peptides intramuscularly, intranasally, or orally in presence or absence of different adjuvants (Poly I:C, Resiquimod, 20 μg/mouse, or heat-killed Caulobacter crescentus HKCC). Details of dose and route of administration are indicated in figures or figure legends. Mice were euthanized 8 days after the first or second immunization(s) and various tissue samples (e.g. spleens, inguinal lymph nodes, ovaries, serum etc.) were collected. All animal experiments were approved by University of Alberta Animal Care and Use Committee in accordance with the Canadian Council of Animal Care guidelines.

Immunohistochemistry.

Twelve, 24 and 48 hours after immunization, mice were euthanized and thigh muscle cells were collected. Ten-micrometer sections of quadriceps muscles of hind limbs of immunized mice were fixed on the slides and stained as follows. Briefly, slides were washed two times with 0.05% Tween phosphate-buffered saline (PBS) buffer for 2 min, followed by cleaning with Triton X-100 containing PBS. Nonspecific binding of biotinylated secondary antibody, used later in the procedure, was blocked by incubation with 5% diluted normal goat serum at room temperature for 30 minutes. Sections were then incubated with anti CD16/32 for one hour followed by two washes with 0.05% Tween PBS. Slides were then incubated with anti-core and anti-NS-3 primary antibody in a 1:100 dilution for 30 minutes followed by two washes with 0.05% Tween PBS. Endogenous peroxidase activity was depleted by incubating the sections in 3% H₂O₂ with 0.1% sodium azide in 0.05% Tween PBS buffer for 10 min. After two washes in 0.05% Tween PBS buffer, sections were incubated with 10 pg/ml of biotinylated goat anti-mouse in 1% normal mouse serum (Sigma) for 20 minutes, and washed twice in 0.05% Tween PBS buffer. The sections were then incubated with DAB for 20 minutes and subsequently washed twice in 0.05% Tween PBS buffer. Chromogen was added to each section for 5 minutes, and washed twice in 0.05% Tween PBS buffer. Sections were dried and dehydrated with 95% and 100% ethanol, cleared with xylene and mounted with water-based plastic mount (Polysciences, Inc.).

T Cell Proliferation Assay.

Eight days after last immunization, mice were euthanized, spleens and/or inguinal lymph nodes were collected. The spleens were pooled from replicates and ground to a single cell suspension and filtered through a Falcon 100 μm nylon cell strainer. The cells were resuspended in 2 ml of media and passed through an equilibrated nylon wool column. The column was washed after 45 min of incubation at 37° C. and the flow through contained the splenic T cells. These T cells were used in the experiments (˜90% CD3⁺ T cells). Lymph nodes were ground into single cell suspension and used in the assays. Proliferative responses were measured in triplicate cultures in 96-well flat-bottomed microtiter plates. A total of 4×10⁵ T cells from immunized mice and 4×10⁵ APCs (spleen cells from control mice irradiated with 18 Gy) were mixed with different HCV derived proteins (Core=c22-3, NS3=c33c; NS4=c100-3; NS5=NS5 SOD, polyprotein c25=Core+NS3+NS4; polyprotein c200=NS3+NS4 or control protein=rhSOD) or synthetic HCV derived peptides (listed in tables 1-5) at different concentrations as described in figure legends. In experiments using rAd containing non-HCV antigens, respective antigens were used. T cell proliferation was assessed by radioactive ³H-thymidine incorporation assay. Detailed methodologies for T cell proliferation assays have been reported previously.

Cytokine ELISA.

Various cytokines (such as IFN-□, IL-10) were assessed in culture supernatants collected from T cell proliferation assays using mouse cytokine ELISA kits supplied by eBiosciences (eBiosciences Inc. San Diego, USA). ELISA was performed according to the manufacturer's instructions manual. Plates were read and data was analyzed in FluoStar ELISA reader (BMG Labtech GmbH, Ortenberg, Germany). Calculated concentration was multiplied with the dilution factor to quantitate the cytokine concentration in per ml of culture supernatant and averages of these concentrations (pg/ml) from duplicate wells were plotted in graphs.

Antibody ELISA.

Serum was prepared from the blood of immunized mice and stored at −20° C. until use. For the detection of HCV antigen specific cross-reactive IgG and IgG1 antibodies in Ad vector immunized mice, 96-well plates were coated with HCV antigens (Core, NS3, NS4 or NS5) at 1 μg/ml in 1×PBS overnight at 4° C. Next day, after blocking with 1% BSA at room temperature for 1 hour, serial dilutions of serum samples were added to 96-well plate in 2-3 replicates and incubated again at room temperature for 2 hours. After application of serum, anti-mouse IgG or IgG1 labeled with alkaline phosphatase (AP) (Southern Biotech, Alabama, USA) was added and plates were incubated for 1 hour, and finally color was developed by adding PNPP substrate (Southern Biotech, Alabama, USA). Plates were washed with 1×PBST (1×PBS with 0.1% Tween-20) after each incubation step. Absorbance was read using FluoStar Optima ELISA Reader (BMG Labtech GmbH, Ortenberg, Germany).

Immunization of Mice with Bone Marrow Derived DCs Infected with Ad Vector.

Female C57 BL/6 mice were euthanized and bone marrow cells were harvested from tibiae and femur bones, and cultured in RPMI-1640 medium supplemented with 10% heat-inactivated fetal bovine serum, 2-mercaptoethanol (50 mM), 400 U/ml murine GM-CSF and 1% Penicillin/Streptomycin at 10×10⁶ cells/100 mm petri dish. Half of the medium was changed on days 3, 6, 8 and 10. On day 12, differentiated DCs were harvested and counted. DCs obtained were further cultured overnight in presence of Ad vector (100 MOI) or media. Twenty-four hours after, cells were washed, counted and a sample of 0.5×10⁶ cells was taken for flow cytometry analysis, and the remaining DCs were used to inject mice. Naïve (media) or Ad vector infected DCs (1.0×10⁶/mouse) were injected subcutaneously. Prior to injection, about 74-83% of the cell population was CD11c⁺. Upon infection with Ad vector, they showed a small up-regulation of maturation markers (MHC class-II and CD86, data not shown). Two immunizations were given on days 0 and 14. Mice were euthanized 8 days after second immunization to evaluate cellular immune responses against HCV antigens.

Flow Cytometry.

Mouse splenocytes were cultured with 5 μg/ml of HCV protein antigens or peptides for 5 days in RPMI-1640 media supplemented with 10% fetal bovine serum. On the fifth day, cells were harvested and counted, and 1×10⁶ cells per group were stained for intracellular Granzyme B (Alexa fluor 647) and extracellular CD8 (APC efluor 780) markers. To perform intracellular cytokine staining, splenocytes cultured for 5 days with HCV antigens were treated with ionomycin (1 μg/ml), phorbol 12-myristate 13-acetate or PMA (50 ng/ml) and brefeldin A (1.5 μg/ml) for 5 hours and subsequently stained for extracellular lineage markers: CD3 (PE Cy7), CD4 (APC) and CD8 (APC efluor 780); and intracellular cytokines: IFN-γ (PE) and IL-10 (FITC). For T cell proliferation using CFSE dilution assay, splenocytes were enriched for T cells using nylon wool column and stained with 1 uM CFSE in 1 ml of cell suspension for 7-10 minutes at room temperature. These CFSE stained cells were washed in 1×PBS containing 10% fetal bovine serum, counted and plated in 24-well plate with 1 μg/ml of HCV NS5 antigen and equal number of γ-irradiated syngeneic splenocytes as APCs. After four days, cells were stained for CD4⁺ and CD8⁺ T cell markers. The cells were run in BD FACS Canto, and data were analyzed using FACS Diva and FCS Express 4.0 softwares. Fluorescently labeled antibodies against various cell markers were purchased from eBiosciences.

T Cell Cytotoxicity Assay:

Spleen T cells harvested from Ad immunized mice were stimulated in vitro with the HCV protein antigens Core, NS3, NS4 or NS5 at 5 μg/ml concentration for 4 days. The target EL4 cells were incubated with corresponding HCV peptides (Core peptides: 2, 14, 17, 25, 27, 28, 32; NS3 peptides: 8, 10; NS4 peptides: 3, 4, 8; and NS5 peptides: 1a, 2a, 16a, 20a, 5b, 19b, 23b, 39b; or All: a mixture of the above peptides from core, NS3, NS4 and NS5) overnight at 37° C. and peptide loaded EL4 cells were cultured with effectors at 10:1 (effectors:target) ratio for 4-5 hours. CFSE labeled live targets were quantified by flow cytometry and subtracted from background CFSE labeled targets to get numbers of killed targets. Empty (no peptide loaded) EL4 targets were used as a negative control.

Chimeric Vac-HCV Challenge.

Eight days after the last immunization with Ad vector or PBS, mice were challenged with 1×10⁷ PFU of Vac-HCV chimeric virus (Vac-Core-NS3 including Core, NS2 and NS3 antigens of HCV or Vac-NS3-NS5 including NS3, NS4 and NS5 antigens of HCV), or wild-type Vaccinia (WT-Vac, not containing HCV antigens) virus intraperitoneally. Five days after virus challenge, mice were euthanized and ovaries were removed, homogenized and freeze-thawed three times in 1×PBS. Homogenized samples were stored at −80° C. until used for viral titer.

Vac-HCV or WT-Vac Titration by Plaque Assay.

Serially diluted samples of ovary homogenates were added in duplicate wells in 6-well plates containing 80% confluent monolayers of TK-1 cells (ATCC # CRL8303) and incubated for 90 minutes. Subsequently, unbound virus was removed and fresh 1×DMEM media (Gibco by Invitrogen, NY, USA) supplemented with 3% FBS, was added and plates were incubated for 48 hours. At this time, media was removed, and plaques were fixed by using 10% formaldehyde (Fisher Scientific, NJ, USA) at room temperature for 30 minutes. Plates were washed with PBS and the monolayers were stained with 0.5% crystal violet (Sigma-Aldrich Company, MO, USA) for 30 minutes, followed by further washing. Plaques were counted, averaged and multiplied with the dilution factor to determine the viral load/mouse.

Statistical Analysis.

Data were analyzed by Graph-pad Prism software (Graph-pad Software Inc., CA, USA). Student t-test was used to determine the significant difference between two groups. p-value less than 0.05 (<0.05) was considered to be statistically significant.

### Example 1

The following example describes the amino acid sequence alignment of adenoviral vector (Ad) proteins from human Ad5 and Chimp Ad25 with the 15 or 20-amino acid long sequences of different HCV proteins to identify the regions of homology in both Ad proteins and HCV proteins by using bioinformatics tool Clustal W.

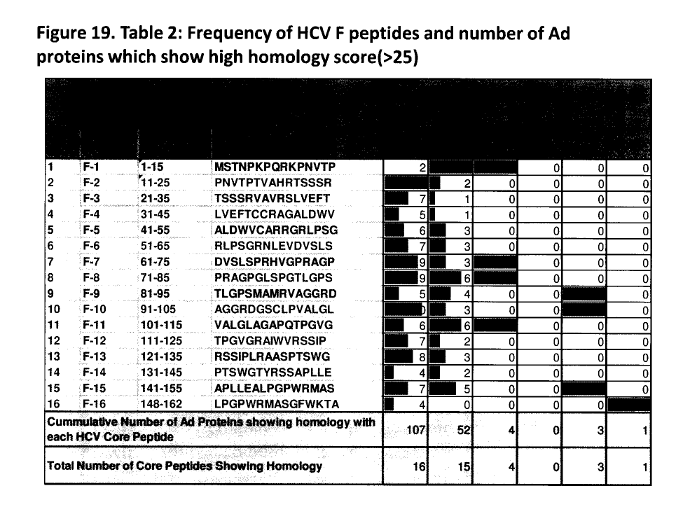

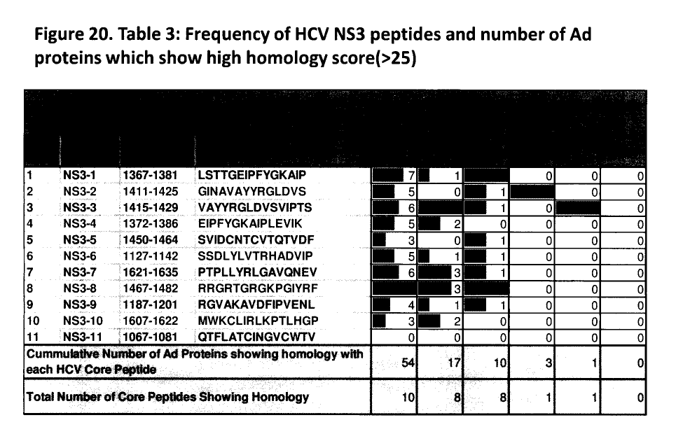

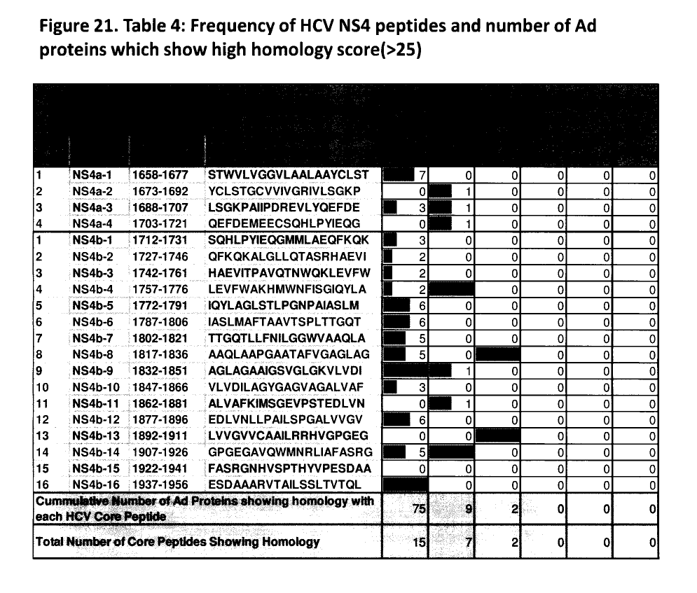

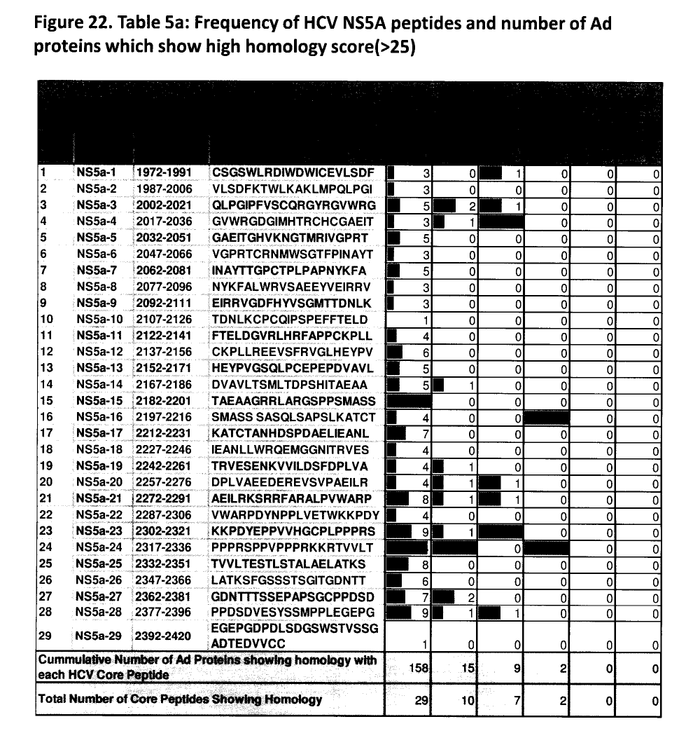

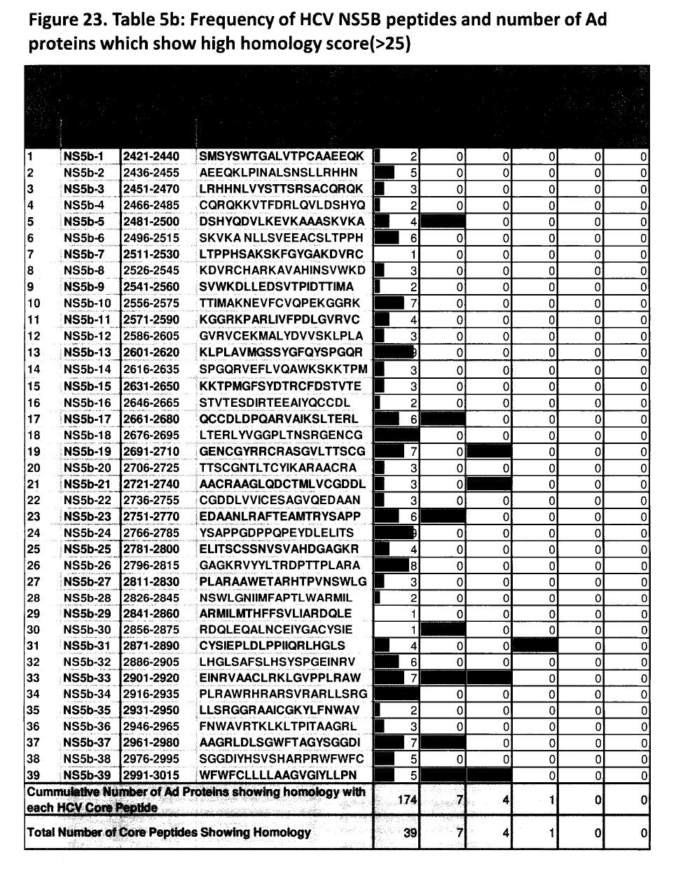

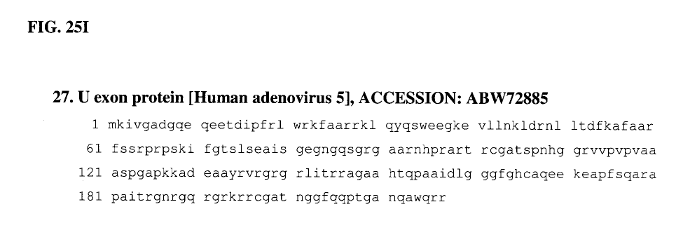

The sequence analyses demonstrate that various Ad proteins show extensive regions of homology with HCV peptides ranging from scores 1-50. FIGS. 18-24 (Tables 1-6) summarize the homology of different HCV peptides with the Ad proteins from human Ad5. We found high homology between HCV peptide sequences from core, F, NS3, NS5 proteins and a large number of Ad proteins. We also found that an individual peptide from various HCV antigens showed homology with multiple Ad proteins. These homologies also resulted in high cross-reactive cellular immune responses against HCV antigens as described in following examples. HCV NS4 derived peptides showed the least homology with the lowest number of Ad proteins (Table 4; FIG. 21). We compared peptide sequences from HCV core with amino acid sequences of various proteins from a simian adenovirus (Chimp Ad25) (FIG. 29, Table 8). Interestingly, there was high level of homology (25-40%) between HCV core peptides and ChAd25 proteins, suggesting that Chimp adenoviruses will also induce HCV cross-reactive immune responses.

### Example 2

PCR Amplification of DNA purified from adenoviral vector (Ad vector) stocks and recombinant Ad vector (rAd) harboring HCV transgenes used in immunization studies.

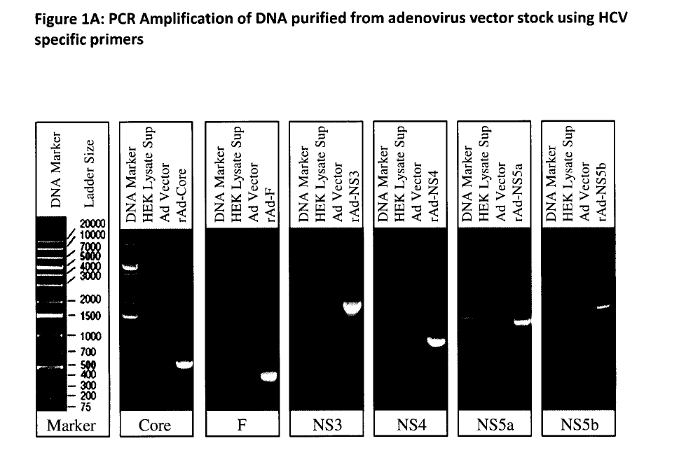

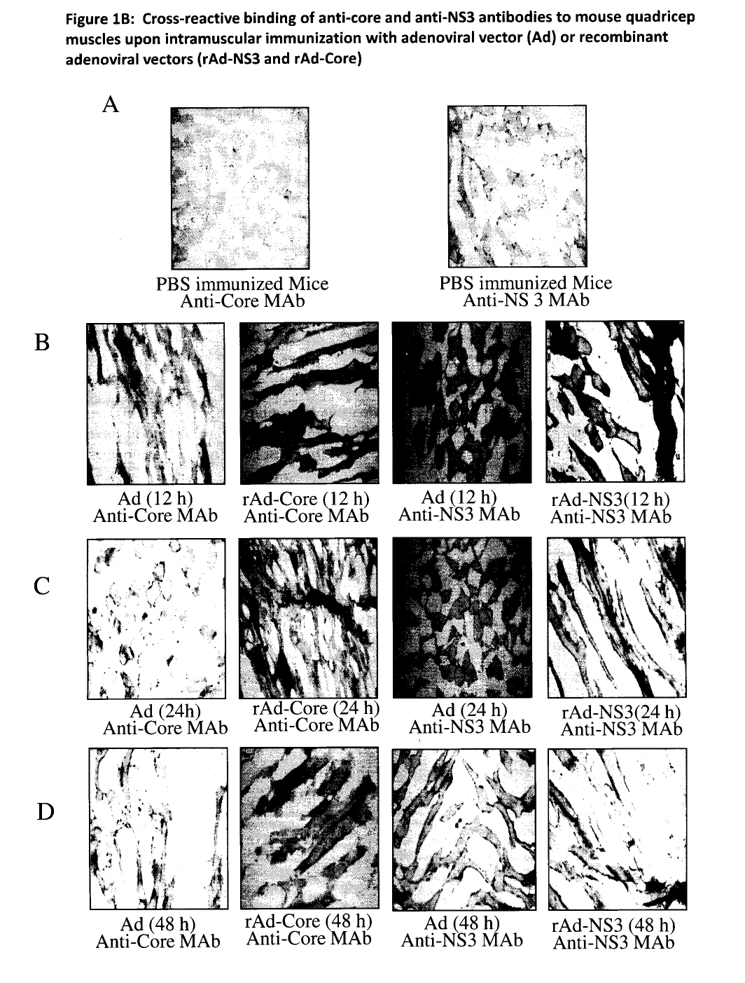

The PCR analysis experiment demonstrates that Ad vector used, which was found to induce both cellular and humoral immune responses against HCV antigens, was devoid of any cross contamination with HCV genes (FIG. 1A). In the PCR reaction products run on 1% agarose gel, none of the primer sets specific for NS3, core, F, NS4, NS5a and NS5b antigens of HCV were able to detect HCV antigen products from Ad vector DNA template and relevant genes in recombinant Ad virus DNAs containing HCV genes were detected. Therefore, Ad vector stock was devoid of any HCV gene contaminations and the cross-reactive immune responses induced in mice against various HCV antigens described in following examples are solely due to heterologous cross-reactive immunity generated by Ad vector expressing adenoviral antigens.

### Example 3

Cross-reactive binding of anti-core and anti-NS3 MAb to quadriceps muscles of mice immunized with Ad or recombinant Ad containing HCV antigens (rAd-core or rAd-NS3).

Mice were immunized with replication deficient adenovector (Ad) or Ad containing

HCV derived core or NS3 proteins (2×10⁷ PFU/mouse) intramuscularly. Twelve, 24, and 48 hours after immunization, mice were euthanized and the quadriceps muscles of the hind limbs were collected for immunohistochemistry. These time points were chosen because we are using replication-incompetent recombinant adenovirus vectors. At all time points, significant cross-reactive binding of Anti-NS3 and anti-core MAbs was observed in Ad group, and positive controls rAd-NS3 and rAd-core immunized groups (FIG. 1B). The intensity of cross-reactive binding was qualitatively lower compared to the relevant transgene-expressing Ads. As negative controls, quadriceps muscles from PBS immunized mice were stained with anti-core and anti-NS3 antibody (FIG. 1B, top panel). Isotype control antibodies did not show any cross-reactive binding.

### Example 4

Induction of cross-reactive T cells, cytokines and antibody responses against HCV antigens (Core, NS3, NS4 and NS5), after two intramuscular (i.m.) immunizations with adenoviral vector (Ad) in the absence or presence of toll like receptor (TLR) agonists.

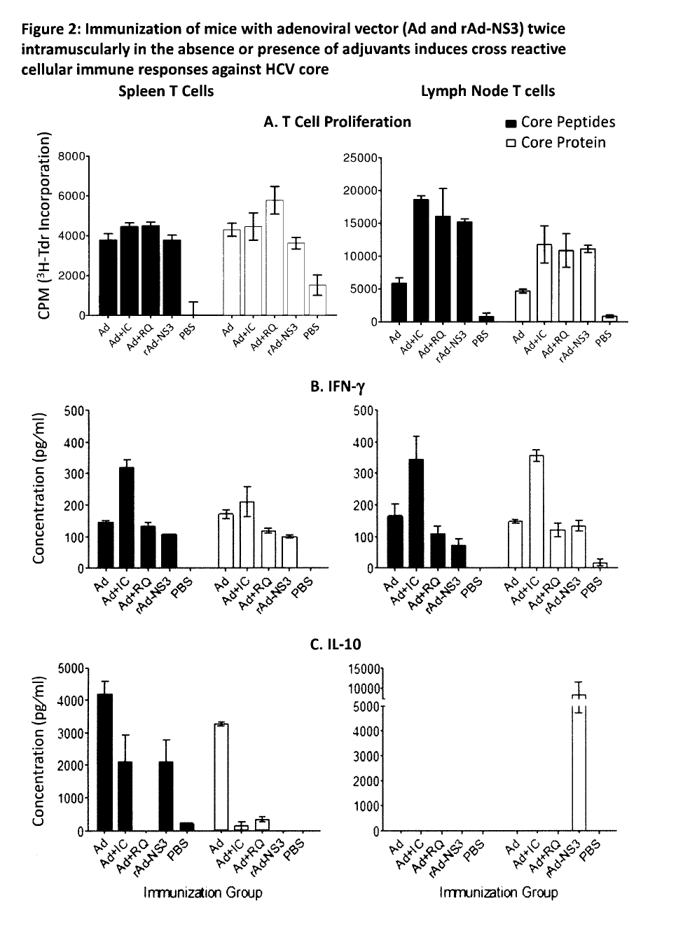

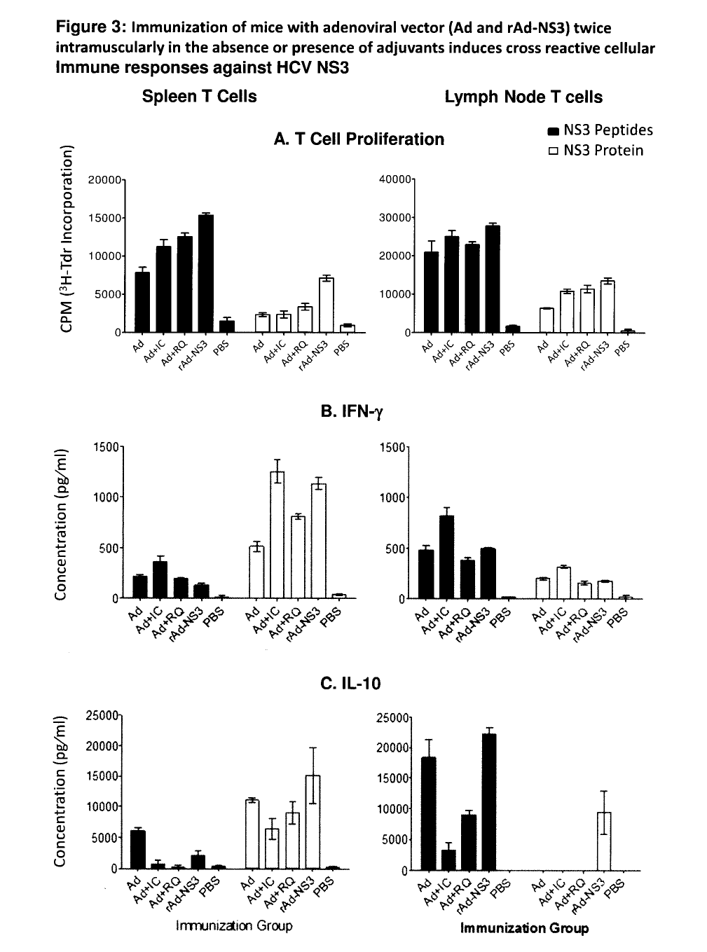

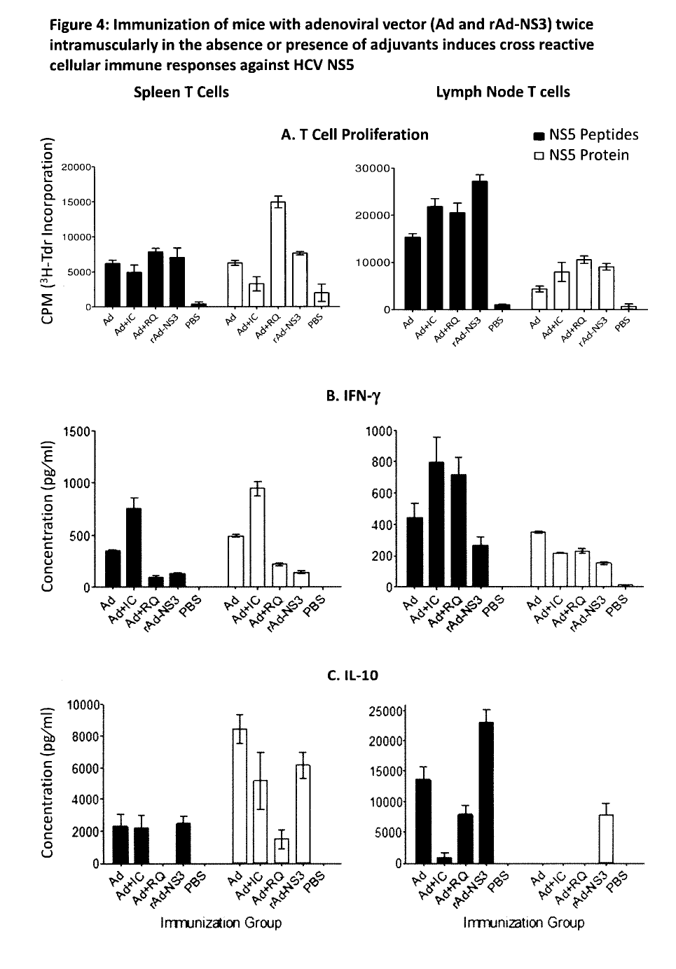

Female C57b/6 mice (n=5/group) were immunized twice (at 14 day interval) intramuscularly with 2×10⁷ PFU/mouse adenoviral vector (Ad), Ad+poly I:C, Ad+resiquimod, rAd-NS3 or PBS. The proliferation of spleen and lymph node T cells obtained from mice was determined against HCV Core, NS3 or NS5 protein antigens or pools of selected peptides from HCV proteins (Core Pool: 5, 14, 16, 17 & 27; NS3 Pool: 5, 6, 8, 15 & 17; NS5 Pool: 5a-6, 24/b-5, 19 & 27) (FIG. 2-4). Both spleen and lymph node T cells from Ad immunized group demonstrated high HCV antigen specific proliferation, which was further increased by co-administration of poly I:C and resiquimod adjuvants. Furthermore, presence of NS3 transgene in Ad vector showed robust cross-reactive proliferation against all of the HCV antigens tested (core, NS3, NS4 and NS5), similar or higher than Ad+poly I:C or Ad+resiquimod groups (FIG. 2A, 3A, 4A). We also analyzed IFN-γ and IL-10 secretion in culture supernatants collected from T cell proliferation assays. In the Ad immunized group, both spleen and lymph node T cells produced IFN-γ in response to various HCV protein antigens and peptide pools, which was significantly increased in the T cells obtained from mice immunized with Ad vector plus poly I:C adjuvant (FIG. 2B, 3B, 4B). IFN-γ levels in culture supernatants upon in vitro stimulation with various HCV proteins or peptides was also significantly higher in rAd-NS3 immunized mice in comparison to PBS immunized mice.

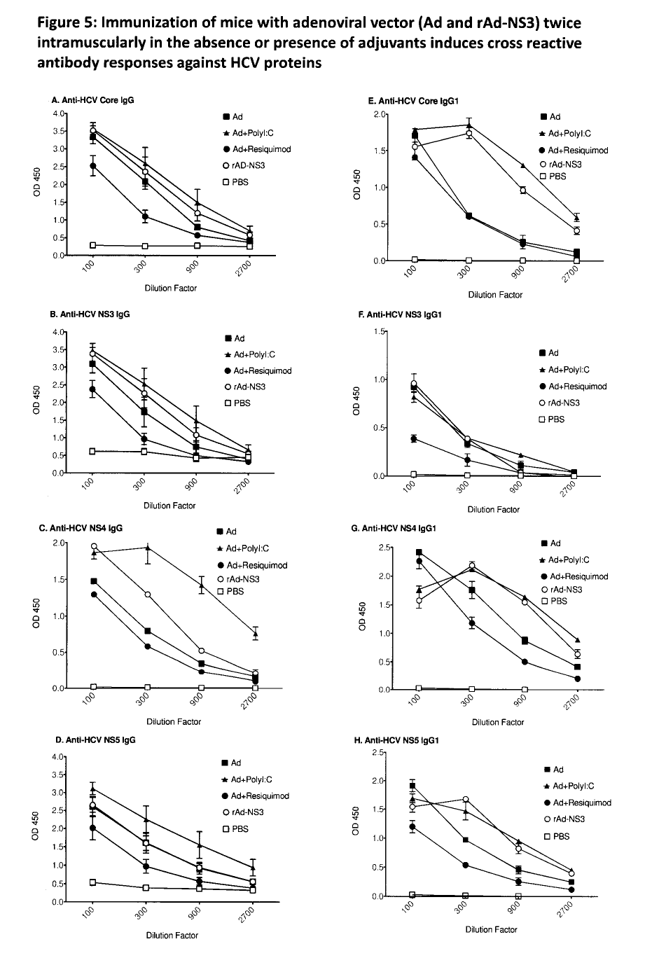

Further, we assessed cross-reactive antibodies against HCV protein antigens (Core, NS3, NS4 and NS5) in the serum samples Ad vector immunized mice (FIG. 5 A-H). Ad vector induced significant amounts of cross-reactive antibodies against various HCV antigens (Core, NS3, NS4 and NS5), which correlated with the T cell proliferation responses. Interestingly, co-administration of TLR agonists poly I:C or presence of HCV NS3 transgene in Ad vector enhanced the levels of anti-HCV Core, NS3, NS4 and NS5 cross-reactive antibodies. PBS immunized mice did not show IgG or IgG1 binding to HCV core, NS3, NS4 and NS5 antigens (FIG. 5).

Unexpectedly, broadly directed and robust T cell and antibody responses against several HCV conserved antigens were observed upon i.m. immunization with replication defective adenoviral vector (Ad). Thus, immunization with Ad virus alone can induce broad, robust multifunctional immune responses against HCV.

Further, recombinant Ad-NS3 unexpectedly demonstrated similar or higher cross-reactive cellular and humoral immune responses against HCV antigens (core, NS3, NS4 and NS5) compared to immunization with transgene free Ad.

We also observed that the cellular immune responses generated against HCV antigens upon immunization with rAd-NS3 were similar or higher then immunization with Ad vector co-administered with poly I:C or resiquimod adjuvants.

### Example 5

Induction of cross-reactive cellular immune responses against HCV antigens (Core, NS3, NS4 and NS5) in mice immunized with adenoviral vector (Ad) through various routes of immunizations.

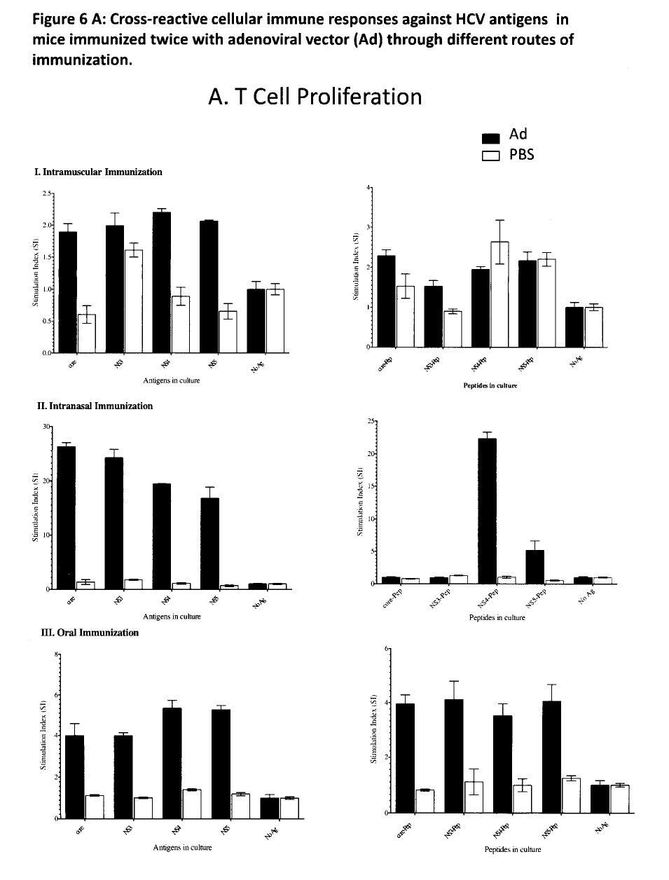

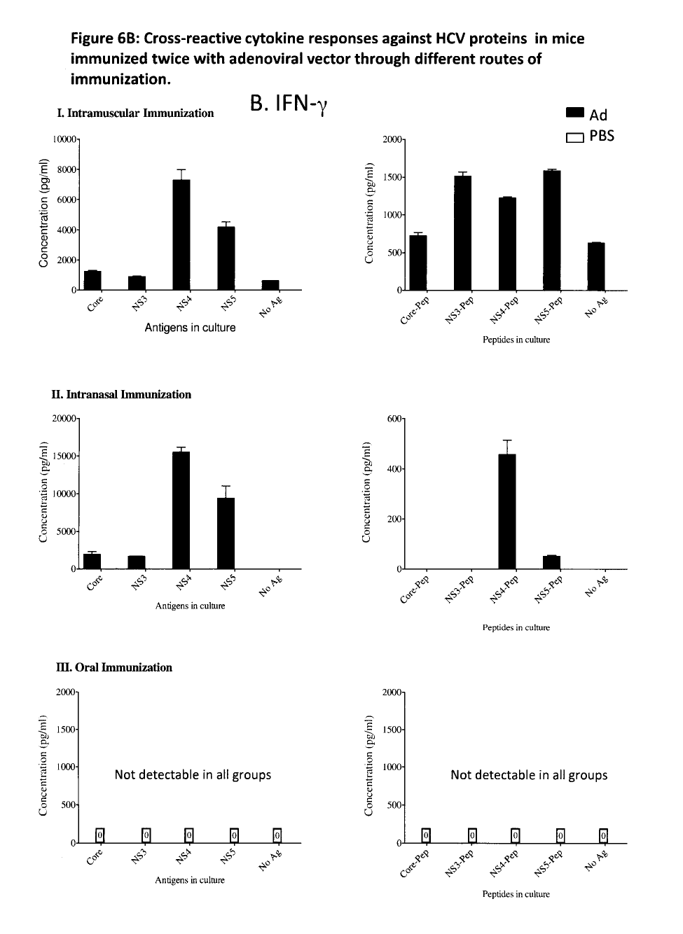

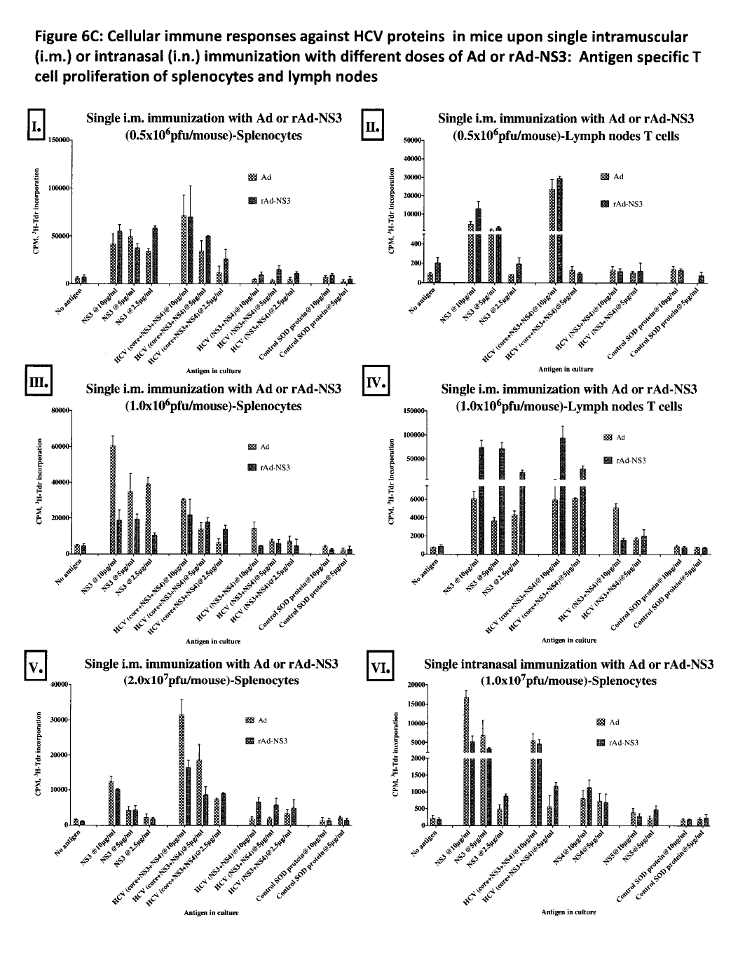

Female C57bl/6 mice were immunized with two doses of Ad vector (2×10⁷ pfu/mouse) or PBS at two weeks' time interval through intramuscular (i.m.), intranasal (i.n.) or oral routes. The proliferation of splenocytes harvested from immunized mice was determined in response to HCV Core, NS3, NS4 or NS5 protein antigens or pools of selected peptides from HCV proteins (Core Pool: 5, 14, 16, 17 & 27; NS3 Pool: 5, 6, 8, 15 & 17; NS4 Pool: 4, 8, 9, 13 & 14; NS5 Pool: 5a-6, 24/b-5, 19 & 27). With all of the routes tested, spleen T cells from Ad immunized mice showed significantly high proliferation in in vitro stimulation with HCV protein antigens (FIG. 6A). We also analyzed IFN-γ secretion in culture supernatant collected from T cell proliferation assay. Spleen T cells produce high amount of IFN-γ cytokine in response to HCV protein antigens and peptide pools in Ad immunized mice via both i.m. and i.n. routes but not by oral route (FIG. 6B). Overall, broadly directed T cell and antibody responses (data not shown) against several HCV conserved antigens were observed upon i.m., i.n. and oral immunization with replication defective adenoviral vector (Ad).

To determine whether single immunization with Ad and rAd-NS3 also induces cellular immune responses against various recombinant HCV proteins, mice were immunized once intramuscularly or intranasally with different doses (0.5×10⁶, 1×10⁶ and 2×10⁷ pfu/mouse) of Ad or rAd-NS3 vector (FIG. 6C). Splenocytes and/or inguinal lymph node T cells were isolated 8 days after immunization and HCV antigens-specific T cell proliferation was determined. At all of the doses tested by i.m. or i.n. route, both Ad and rAd-NS3 vector induced robust HCV specific T cell proliferative responses in spleens and/or lymph nodes (FIG. 6C).

### Example 6

Cross-reactive T cell responses against a large number of synthetic peptide epitopes of various HCV proteins in mice after intramuscular immunization with adenoviral vector (Ad).

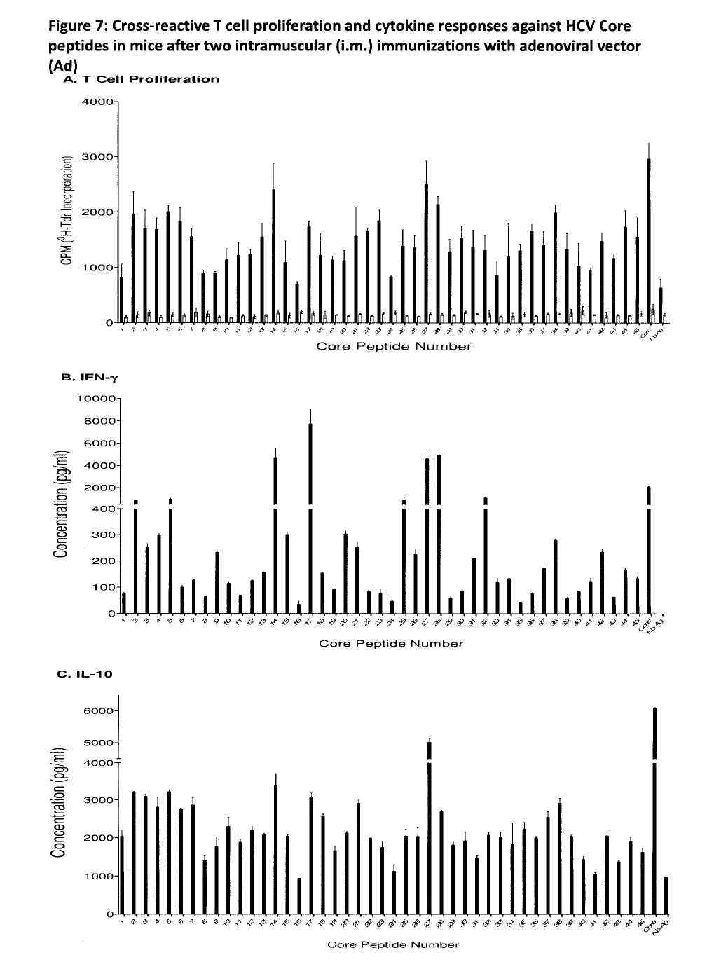

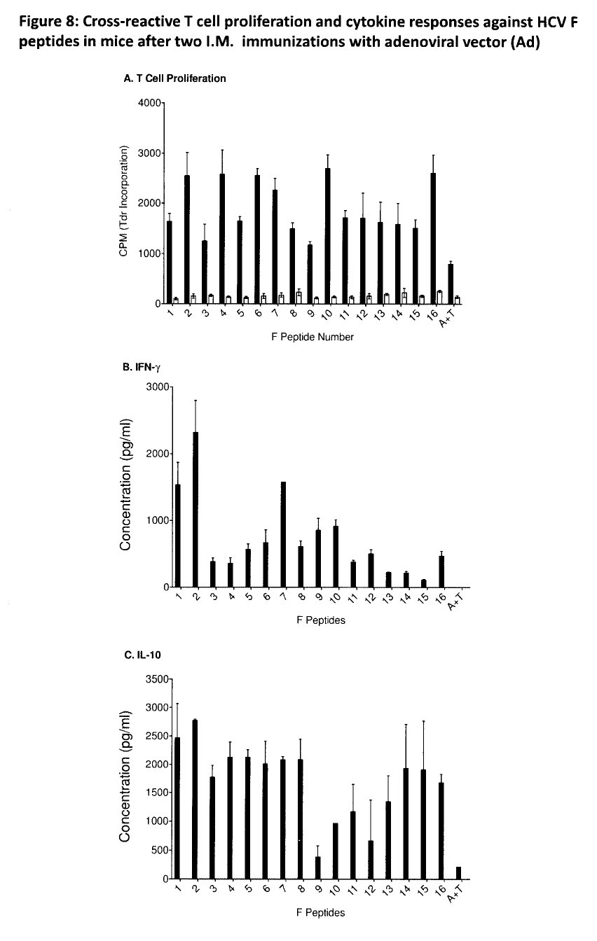

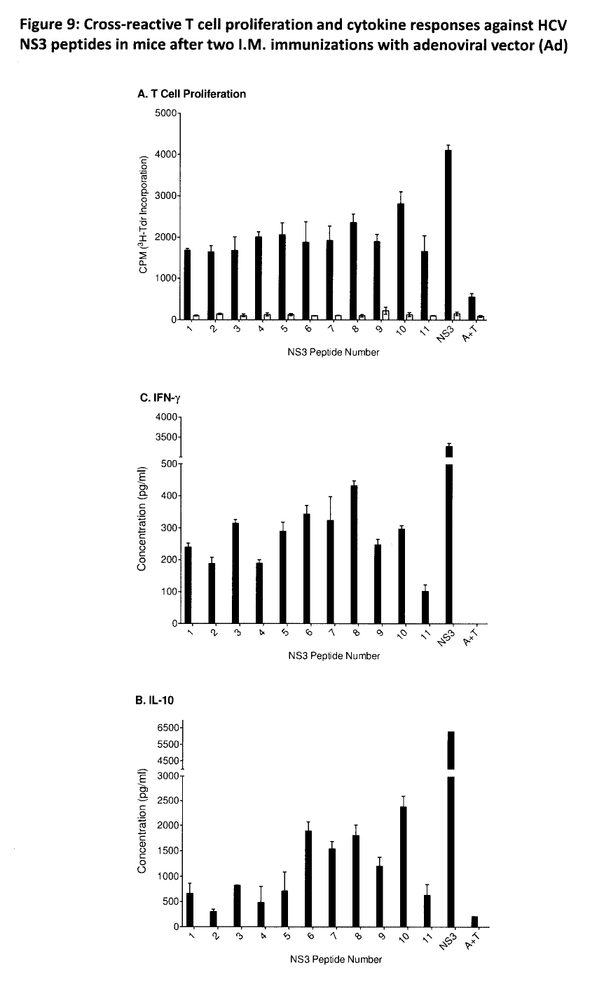

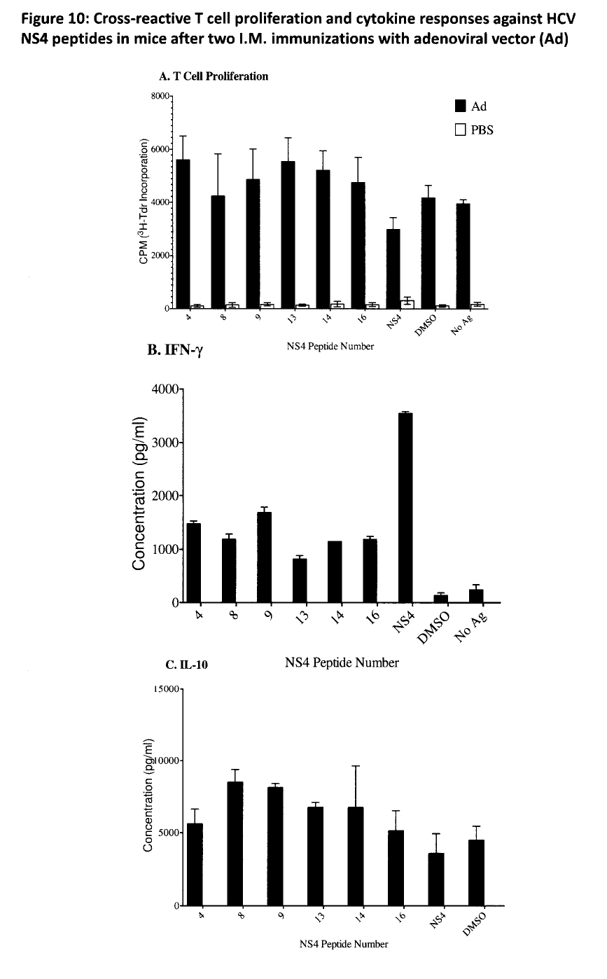

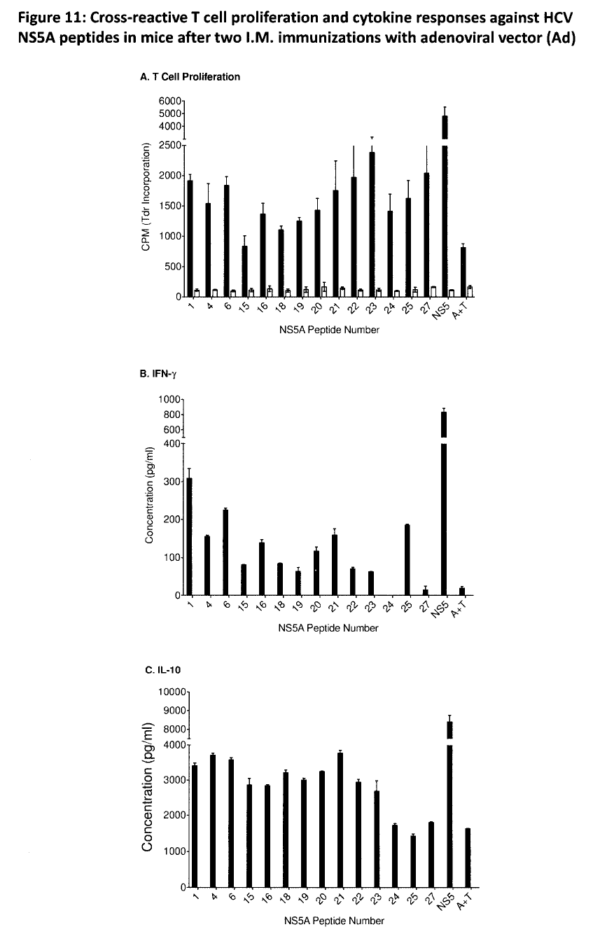

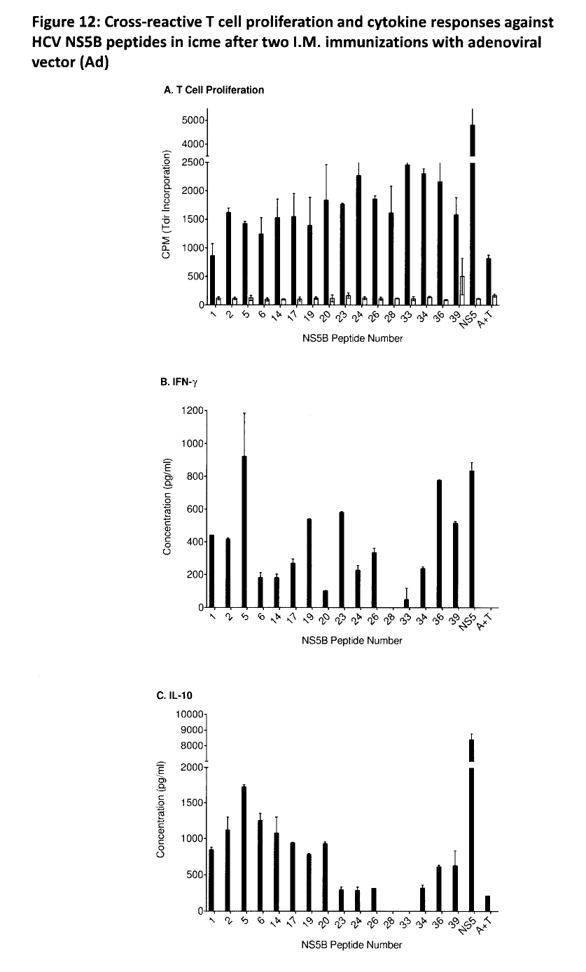

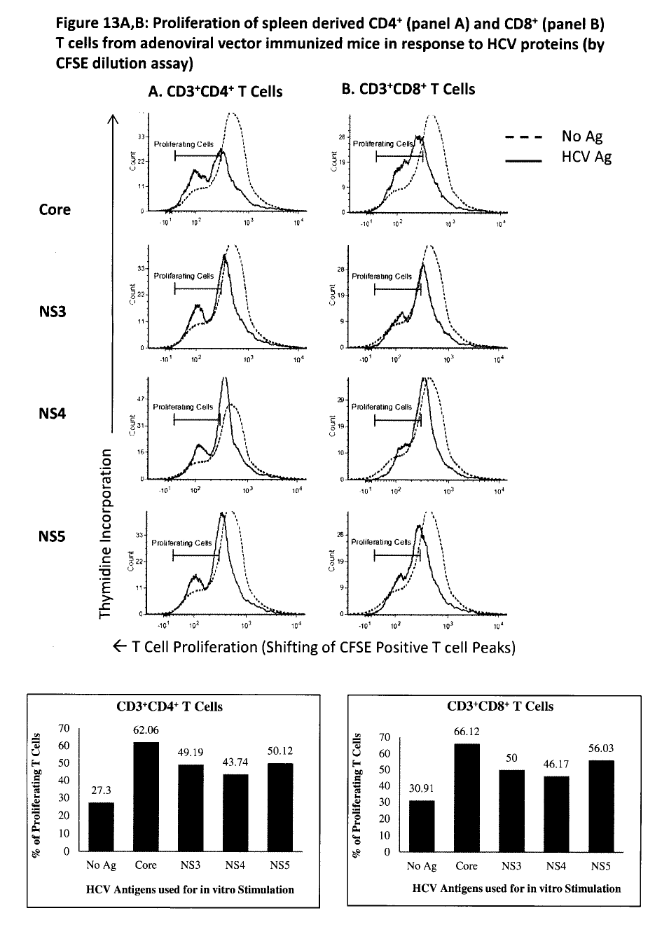

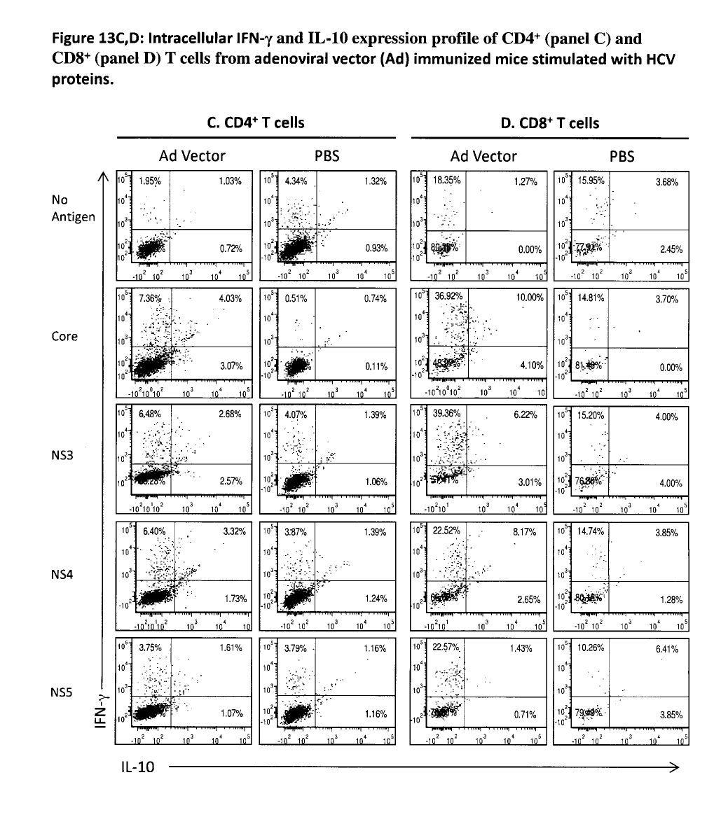

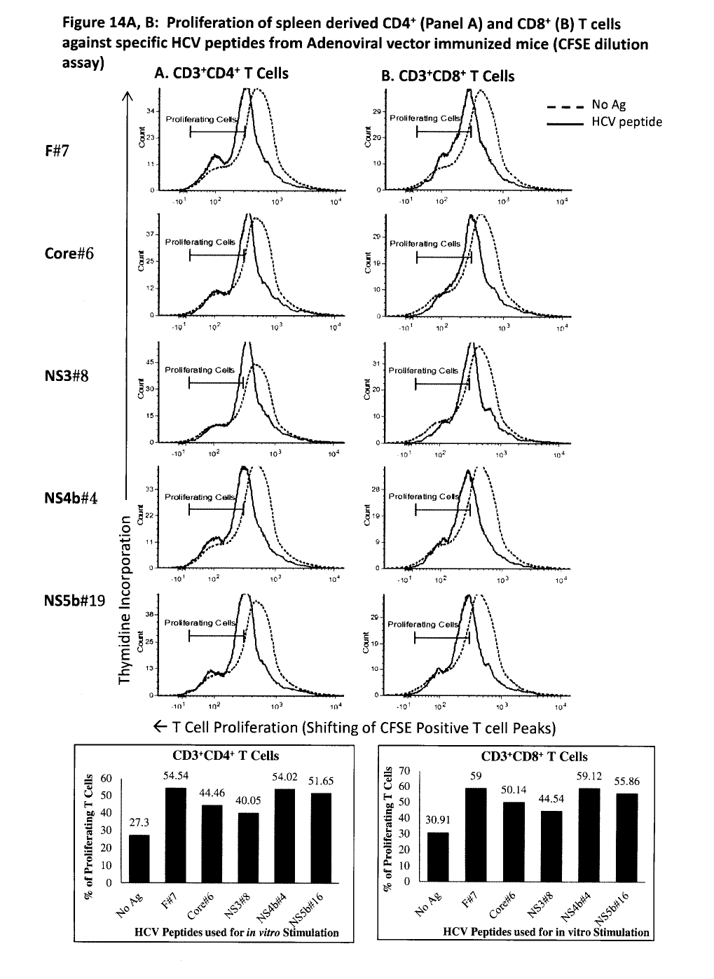

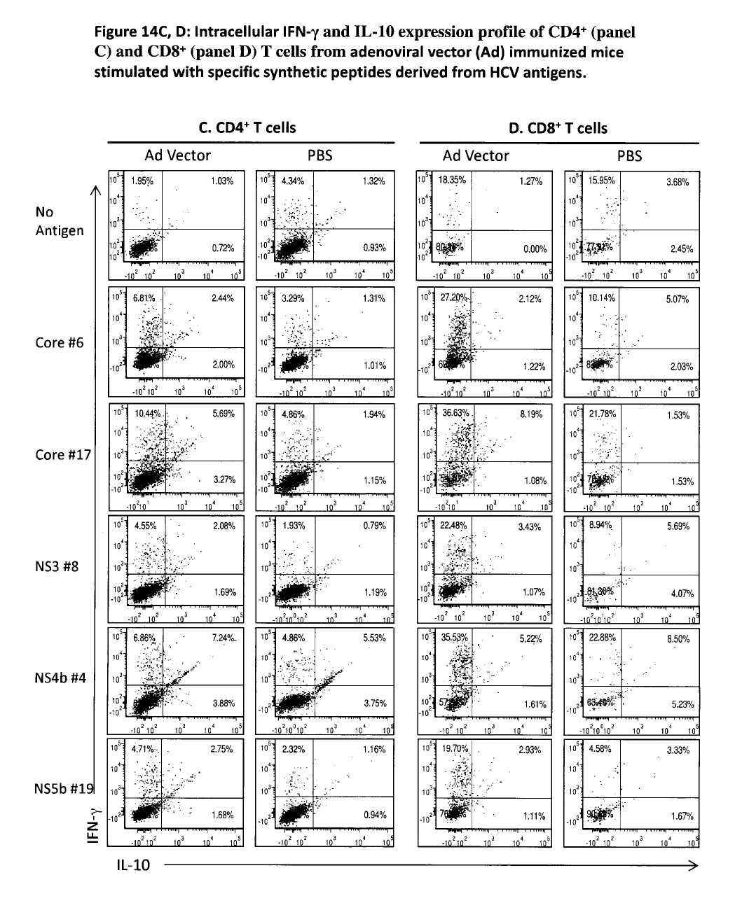

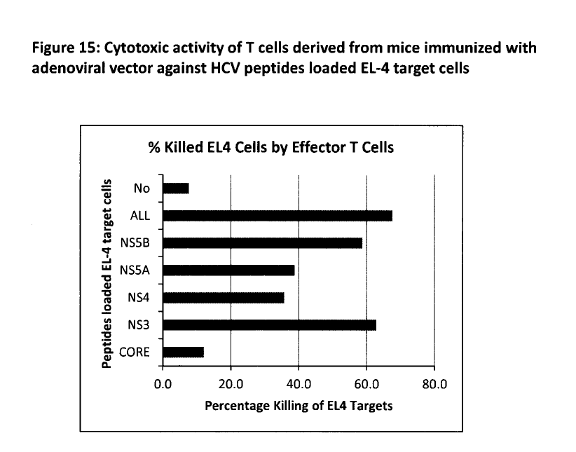

To characterize and identify the domains of cross-reactivity in various HCV antigens with respect to amino acid sequences, we immunized mice with Ad vector and compared spleen T cell proliferation in response to HCV Core, F, NS3, NS5a and NS5b derived synthetic individual peptides with un-immunized (PBS) mice. Several of the HCV Core, F, NS3, NS5a and NS5b peptides are able to induce T cell proliferation ex vivo (FIG. 7-12), which also translated in to production of IFN-γ and IL-10 (FIG. 7-12). Several peptides, which possess a very high amino acid sequence homology with the different Ad vector proteins and with multiple high scoring regions in adenovirus proteins showed T cell proliferation and IFN-γ production. However, some HCV peptides which showed high homology with respect to high score (>35) and number of regions in Ad proteins (Table 1-5; provided in FIGS. 18-23), did not show cross-reactive responses in mice immunized with Ad vector. This could be explained on the basis of homology in TCR contact vs. the non-contact amino acids of the peptides or the overall immunogenicity of those epitopes. Further, to confirm that the high proliferation responses observed in ³H-Tdr assay are due to actual proliferation of HCV specific CD4 and CD8 T cells, we performed CFSE proliferation assay along with staining for CD3, CD4 and CD8, which allows one to demonstrate actual proliferating cells in response to a given protein (FIG. 13A, B) or peptide (FIG. 14 A, B) antigen. Spleen T cells obtained from Ad vector immunized mice and stimulated ex vivo with various recombinant HCV protein antigens (Core, NS3, NS4 and NS5, FIG. 13A, B); and selected representative peptides from these proteins (FIG. 14A,B), showed antigen dependent proliferation of cross-reactive CD4⁺ and CD8⁺ T cells.

Further, to demonstrate that the cytokines produced in culture supernatants observed in experiments described in FIGS. 2-6 are from CD4⁺ and CD8⁺ T cells, we performed intracellular cytokine expression analyses of spleen T cells obtained from Ad vector immunized mice and stimulated ex vivo with various recombinant HCV protein antigens (Core, NS3, NS4 and NS5); or selected representative peptides from HCV proteins. Both CD4⁺ and CD8⁺ T cells from Ad vector immunized mice showed increased expression of IFN-γ upon stimulation with HCV protein antigens (FIG. 13 C, D) or peptide antigens (FIG. 14 C, D) in comparison to PBS immunized mice. T Cells expressing both IFN-γ and IL-10 simultaneously were also in higher frequency in Ad vector immunized mice when stimulated with HCV core, NS3 or NS4 antigens, except NS5 antigen. HCV core stimulated CD4⁺ and CD8⁺ T cells show high frequency of IL-10 expressing cells compared to other HCV antigens. HCV NS5 antigen did not increase IFN-γ production in CD4⁺ T cells; however in CD8⁺ T cells it was significantly increased in Ad vector immunized mice compared to PBS control (FIG. 13 C, D).

Intracellular cytokine analyses were also performed in splenic T cells obtained from Ad vector or PBS immunized mice and cultured with selected representative peptides derived from various HCV antigens core, NS3, NS4 and NS5 (FIG. 14 C, D). Cross-reactive CD4⁺ T cells from Ad immunized mice showed enhanced IFN-γ and IL-10 expression upon stimulation with all of the peptides tested. Interestingly, frequency of CD4⁺ T cells which express both IFN-γ and IL-10 was also higher in comparison to PBS group. Further, IFN-γ producing CD8⁺ T cells were significantly high with all the peptides used for in vitro stimulation (FIG. 14C, D).

The CFSE proliferation data and intracellular cytokine analyses described in FIGS. 13 and 14 provided conclusive evidence that CD4⁺ and CD8⁺ T cells obtained from Ad vector immunized mice are highly cross-reactive against various HCV antigens and peptides derived from them.

Multi-antigen specific CD4⁺, CD8⁺ T cells, effector cells producing GrB and IFN-γ against highly conserved HCV core, NS3, NS4 and NS5 antigens were also observed.

T cell immunity against conserved epitopes of adenoviruses has been shown to be conserved across various human serotypes and also across species. It was observed that the homology between HCV epitopes and adenoviruses spans across a number of adenoviral proteins including conserved antigens, and thus it can be extended to a number of human (e.g., Human Ad5, Ad6, Ad24 and Ad35) and non-human adenoviruses (such as Chimpanzees Ad3, Bovine etc.). Therefore, cross-reactive heterologous immunity against HCV antigens epitopes may be induced by a number of different rare human and non-human adenoviruses. In this regard, the adenovirus used to induce immunity against HCV could be a bovine adenovirus, a canine adenovirus, non-human primate adenovirus, a chicken adenovirus, a porcine adenovirus, a swine adenovirus, an adeno associated virus or a helper dependent adeno virus, and their various serotypes (e.g., 57 different serotypes from 7 subtypes of human adenoviruses).

### Example 7

Cytotoxic activity of the T cells derived from mice immunized with adenoviral vector (Ad) against EL4 target cells loaded with HCV peptide antigens.

The cytotoxic activity of the cross-reactive effector T cells, obtained from spleens of Ad vector immunized mice and stimulated in vitro (4 days) with 5 μg/ml concentration of HCV protein antigens (Core, NS3, NS4, NS5 or polyprotein), was examined against EL4 targets cells loaded with pools of respective HCV antigen peptides. Briefly, different sets of CFSE stained EL4 targets were prepared by loading them with pools of peptides obtained from different HCV antigens as follows: Core (peptide #2, 14, 17, 25, 27, 28, 32), NS3 (peptides #8, 10), NS4 (peptides #3, 4, 8), NS5 (peptides # NS5a: 1, 2, 16, 20 and NS5b: 5, 19, 23, 39), all of the above peptides (ALL) or no peptide loaded (No). These different EL4 targets were cultured for 4-5 hours with corresponding effector T cells stimulated with HCV protein antigens. The results obtained demonstrated that T cells induced after Ad vector immunization act as potent effector T cells, which can kill HCV peptide antigens loaded EL4 target cells in an antigen specific manner (FIG. 15). Therefore, Ad vector immunization can lead to the induction of strong cytotoxic effectors T cells, which demonstrate HCV antigen specific killing of target cells (FIG. 15).

### Example 8

This example illustrates the use of dendritic cells infected with adenoviral vector (Ad) to induce cross-reactive immune responses against HCV proteins as cellular vaccines.

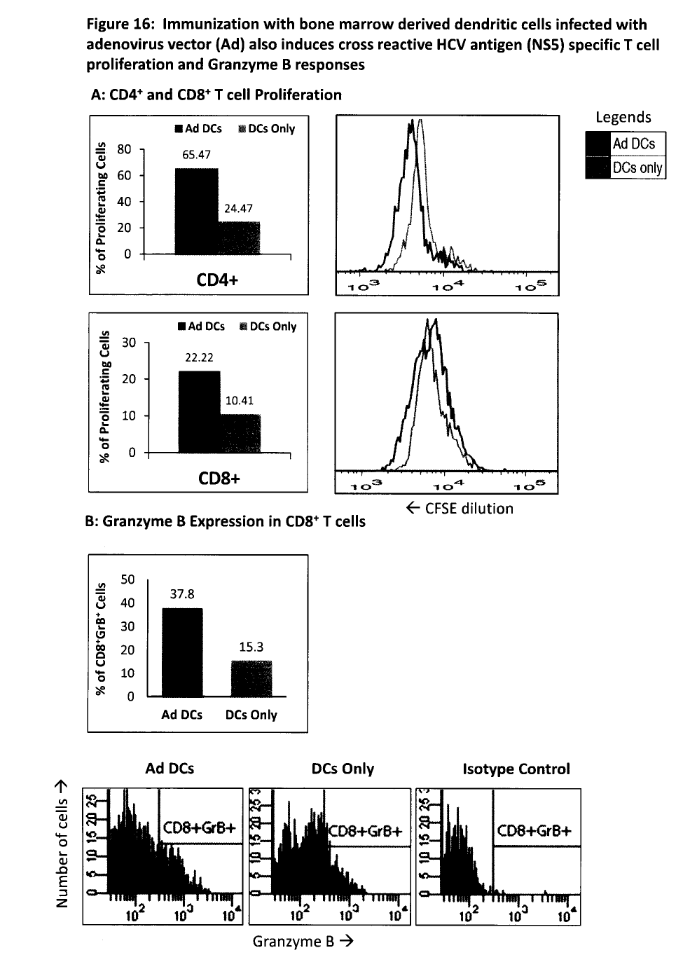

The cross-reactive immune responses against HCV antigen NS5 were examined after immunizations with bone marrow derived dendritic cells (DCs) infected with Ad vector. Mice immunized with uninfected DCs were used as control. Briefly, after 8 days of two subcutaneous immunizations with Ad vector expressing DCs (Ad DCs) or uninfected DCs (DCs only), spleen T cells were harvested, labeled with CFSE, and cultured for five days in the presence of NS5 antigen (5 μg/ml) and APCs. Proliferation of CD4⁺ and CD8⁺ T cells and granzyme B expression by CD8⁺ T cells was evaluated by flow cytometry (FIG. 16). The results demonstrated that immunization with Ad vector infected DCs generated HCV NS5 cross-reactive CD4⁺ and CD8⁺ T cells (FIG. 16A). Further, CD8⁺ T cells also showed high granzyme B expression when restimulated with HCV NS5 protein (FIG. 16B). These results indicate that cross-reactive immune response against HCV antigen is also induced by cellular immunization with DCs infected with Ad vector.

Unexpectedly, Ad induces cross reactive HCV antigen specific CD4⁺ and CD8⁺ T cell responses upon immunization with DC infected with Ad virus ex vivo.

### Example 9

This example describes the role of HCV specific cross-reactive immune responses induced by adenoviral vector (Ad) in the reduction of viral loads in Vaccinia-HCV challenged mice.

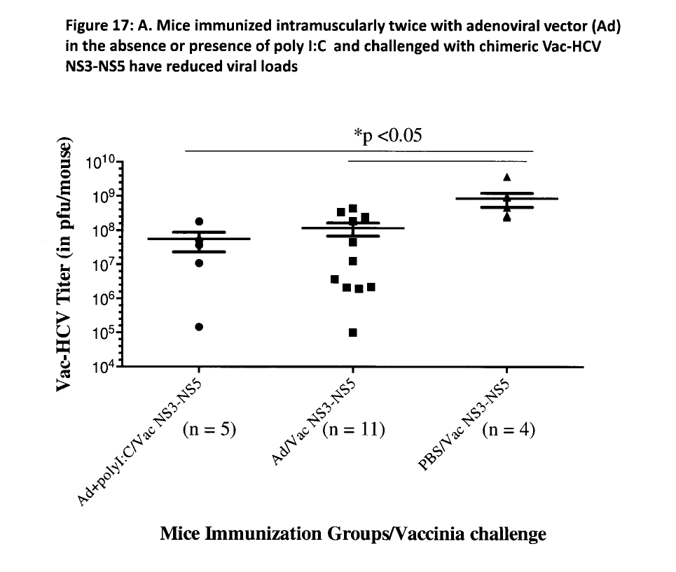

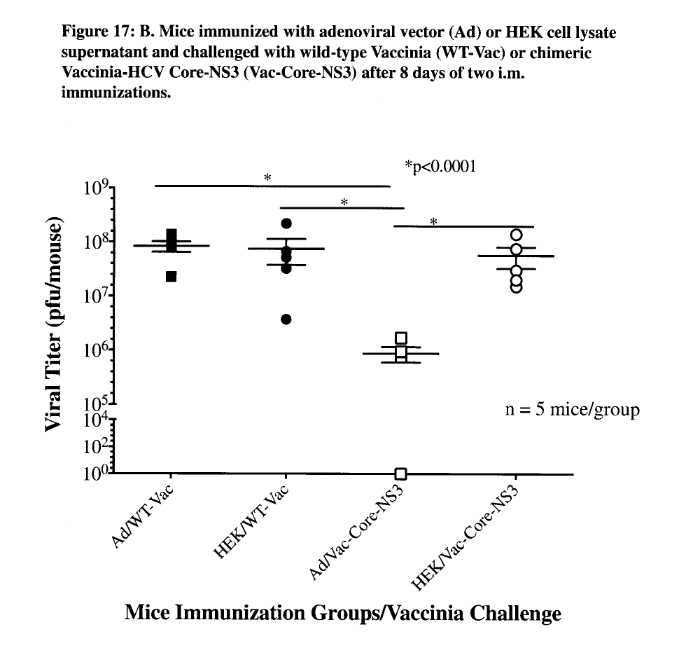

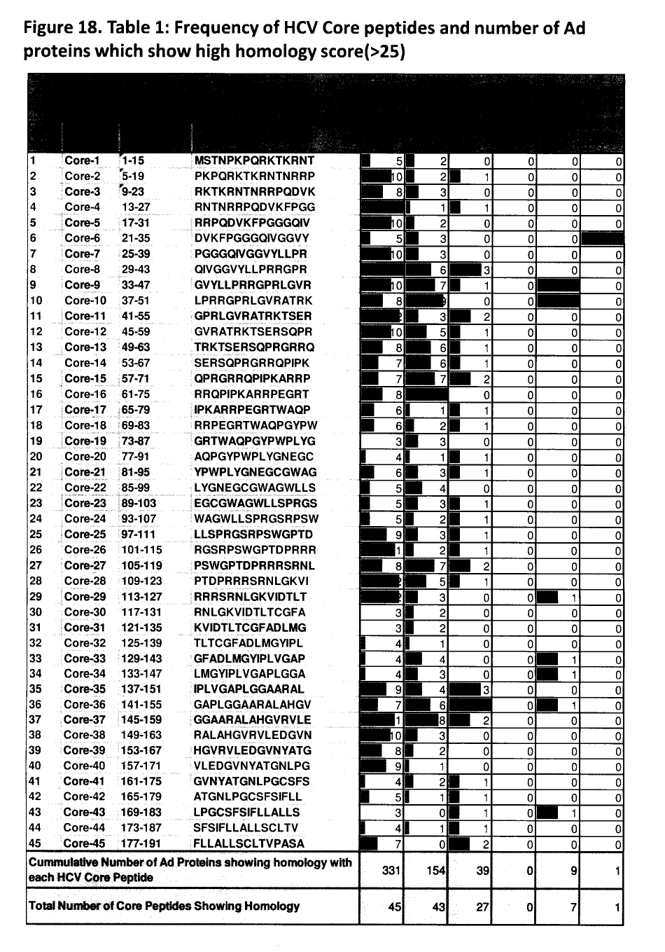

To demonstrate the protective potential of Ad induced cross-reactive immune responses against HCV infection, a surrogate Vac-HCV infection model was used. The recombinant HCV-vaccinia infected mouse model has been reported as a surrogate small animal model for HCV infection. Although this model does not demonstrate exact features of HCV infection, it can be used to assess the ability of the induced immunity to kill targets expressing HCV antigens, a critical parameter in the antiviral response against HCV. Briefly, C57bl/6 mice after two intramuscular immunizations with Ad vector with or without poly I:C as adjuvant, were challenged intraperitoneally with Vaccinia-HCV (Vac-NS3-NS5) chimeric virus. After 5 days of challenge, mice were euthanized and viral loads in ovaries of individual mice were evaluated (FIG. 17 A). The results obtained clearly demonstrate that Ad vector immunized mice had significantly reduced viral loads in comparison to PBS immunized mice, and mice immunized with Ad vector in presence of poly I:C had further reduced viral loads. Therefore, Ad vector immunization can induce antiviral cross-reactive immune response against HCV infection (FIG. 17 A).

To further prove the role of antigen specific heterologous immunity and exclude the possibility of non-specific immunity in viral reduction in HCV-vaccinia challenge model, we used recombinant Vac-HCV (Vac-Core-NS3) or WT-Vac (wild-type vaccinia not including sequences from HCV genome) infection models. Briefly, two groups of mice (n=5/group) were immunized twice with Ad vector (2×10⁷ pfu/mouse) and other two groups (n=5/group) with HEK lysate (cell line used to cultivate Ad, as control) intramuscularly. Each immunized group of mice was challenged with WT-Vac or Vac-HCV (1×10⁷ pfu/mouse, intraperitoneally). Five days after the virus challenge, ovaries were harvested from individual mouse and viral titers were determined using plaque assay in TK-1 cells (FIG. 17B). Immunization with Ad vector led to significant reduction in viral titers in mice infected with Vac-HCV (p<0.05, Student's t test) in contrast to mice infected with WT-Vac (p>0.05), providing conclusive evidence that immunization with Ad leads to cross protective immunity against HCV antigens allowing the reduction in viral titer of Vac-HCV and not WT-Vac. Immunizations with HEK lysate did not lead to significant changes in viral titers in WT-Vac or Vac-HCV infected mice.

### Example 10

This example illustrates the induction of cross-reactive T cell responses against HCV antigens (core, NS3, NS4 and NS5), after two intranasal (i.n) immunizations with recombinant adenoviral vector expressing HIV nef protein (rAd-nef) in the absence or presence of adjuvant Poly I: C.

Male C57bl/6 mice (n=5/group) were immunized twice (at 14 day interval) intranasally with 2×10⁷ PFU/mouse recombinant adenoviral vector expressing HIV-nef (rAd-nef), rAd-nef+poly I:C or PBS. After 8 days of second immunization the proliferation of spleen T cells obtained from mice was determined against HCV Core, NS3, NS4 or NS5 protein antigens and HIV-nef protein (FIG. 26). Splenic T cells from rAd-nef immunized group demonstrated robust cross-reactive proliferation against all of the HCV antigens tested (core, NS3, NS4 and NS5) and HIV antigen specific proliferation, which was further increased by co-administration of poly I:C adjuvant comparison to PBS immunized mice. Therefore, recombinant Ad vector expressing multiple antigens from other viral or cancer-associated antigens can induce cross-reactive HCV-specific immunity and can be used as a single multi-pathogen vaccine to protect and/or prevent two or more disease.

### Example 11

This example demonstrates the induction of cross-reactive T cell responses against HCV antigens (core, NS3 and NS4), after two intramuscular (i.m) immunizations with recombinant adenoviral vector expressing mycobacteria antigen 85B (rAd-Ag85B) or transgene free Ad.

Male C57bl/6 mice (n=5/group) were immunized twice (at 14 day interval) intramuscularly with 2×10⁷ PFU/mouse recombinant adenoviral vector expressing Mycobacterial antigen 85B (rAd-Ag85B), or Ad vector. After 8 days of second immunization the proliferation of spleen T cells obtained from mice was determined against HCV Core, NS3 or NS4 protein antigens and sonicated-mycobacteria at different concentrations (FIG. 27). Splenic T cells from rAd-Ag85B immunized group demonstrated robust cross-reactive proliferation against all of the HCV antigens tested (core, NS3 ad NS4) and mycobacterial antigen specific proliferation, while T cells obtained from Ad vector immunized group showed only HCV-specific T cell responses and Mtb-specific T cell responses were absent in Ad vector group. Therefore, recombinant Ad vector expressing antigens from other bacterial/parasites antigens can induce cross-reactive HCV-specific immunity and can be used as a single multi-pathogen vaccine to protect and/or prevent bacterial and viral disease.

### Example 12

This example demonstrates the protective potential of heterologous priming with Ad vector (i.m) and boosting with pool of HCV-NS3 peptide antigens (i.n) with immunomodulator heat-killed Caulobacter crescentus (HKCC) in the reduction in viral loads in Vaccinia-HCV challenged mice.

Female C57bl/6 mice were first immunized intramuscularly with Ad vector. After 14 days interval, mice were boosted with a pool of HCV-NS3 peptides with HKCC intranasally or PBS. Each immunized group of mice was challenged intraperitoneally with Vaccinia-HCV (Vac-Core-NS3) chimeric virus. Five days after the virus challenge, ovaries were harvested from individual mouse and viral titers were determined using plaque assay in TK-1 cells (FIG. 28). Priming with Ad vector and boosting with NS3 peptides and HKCC [Ad (i.m)-Peptides+HKCC (i.n)] led to significant reduction in viral titers in mice infected with Vac-HCV (p<0.05, Student's t test) in contrast to PBS immunized mice.

The results obtained demonstrate that the potential of cross HCV specific immunity with adenoviral vector can be further enhanced with the use of structural or non-structural HCV peptide/protein antigens with or without an immunomodulator or viral/bacterial vector expressing HCV antigens.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of inducing an immune response in an individual to a hepatitis C virus (HCV) protein, the method comprising administering to the individual an effective amount of an immunogenic composition comprising an adenoviral nucleic acid or an adenovirus polypeptide.

2. The method of claim 1, wherein the adenoviral nucleic acid or adenovirus polypeptide is administered via an oral, intranasal, subcutaneous, transdermal, intratracheal, rectal, intramuscular or parenteral route of administration.

3. The method of any one of claims 1-2, wherein the adenoviral nucleic acid or adenovirus polypeptide is administered multiple times.

4. The method of claim 3, wherein the multiple administrations comprise a first administration wherein the adenoviral nucleic acid or adenovirus polypeptide is a first adenovirus serotype or subtype, and at least a second administration wherein the adenoviral nucleic acid or adenovirus polypeptide is a second adenovirus serotype or subtype.

5. The method of any one of claims 1-4, wherein said immune response comprises a humoral and/or a cellular immune response.

6. The method of any one of claims 1-4, wherein the adenoviral nucleic acid is a full-length adenovirus nucleic acid or an adenovirus nucleic acid comprising a deletion.

7. The method of any one of claims 1-6, wherein the adenoviral nucleic acid does not encode a non-adenovirus polypeptide.

8. The method of any one of claims 1-5, wherein the adenoviral nucleic acid comprises a nucleotide sequence encoding one or more HCV polypeptides.

9. The method of any one of claims 1-5, wherein the adenoviral nucleic acid comprises a nucleotide sequence encoding an antigen associated with a pathogen other than HCV or comprises a nucleotide sequence encoding a cancer-associated antigen.

10. The method of any one of claims 1-6, 8, and 9, wherein the adenoviral nucleic acid comprises a nucleotide sequence associated with an immunostimulatory or immunomodulatory sequence.

11. The method of claim 9, further comprising simultaneously administering a non-recombinant adenovirus.

12. The method of claims 1-9, further comprising administering a structural or a non-structural HCV polypeptide or a nucleic acid comprising a nucleotide sequence encoding the structural or non-structural HCV polypeptide.

13. The method of claim 12, wherein the HCV polypeptide is one or more of E1, E2, F, core, P7, NS2, NS3, NS4 and NS5.

14. The method of claim 12, wherein the structural or non-structural HCV antigen is administered before the adenovirus nucleic acid or the adenovirus polypeptide.

15. The method of claim 12, wherein the structural or non-structural HCV antigen is administered after the adenovirus nucleic acid or the adenovirus polypeptide.

16. The method of any one of claims 1-15, wherein the immunogenic composition comprises an adjuvant.

17. The method of any one of claims 1-15, wherein the composition comprises a cytokine and/or an antibody.

18. The method of any one of claims 1-15, wherein the composition comprises adenoviral nucleic acid or adenovirus polypeptide from two or more different serotypes or subtypes of adenovirus.

19. A method of inducing an immune response in an individual to a hepatitis C virus antigen, the method comprising:
a) obtaining dendritic cells (DCs) from the individual;
b) genetically modifying the DCs to express one or more adenoviral proteins; and
c) administering the genetically modified DCs to the individual.

20. A method of inducing an immune response in an individual to a hepatitis C virus antigen, the method comprising:
a) obtaining DCs from the individual;
b) infecting the DCs with replication competent adenovirus or replication-defective adenovirus; and
c) administering the infected DCs to the individual.

21. A method of inducing an immune response in an individual to a hepatitis C virus antigen, the method comprising:
a) obtaining DCs from the individual;
b) introducing one or more adenoviral proteins, or nucleic acids encoding one or more adenoviral proteins, into the DCs, thereby generating adenoviral protein-expressing DCs; and
c) administering the adenoviral protein-expressing DCs to the individual.

22. A method of treating a hepatitis C virus (HCV) infection in an individual, the method comprising inducing an immune response to one or more HCV antigens in the individual, wherein said inducing comprises a method of any one of claims 1-21.

23. The method of claim 22, comprising administering to the individual an effective amount of at least a second therapeutic agent that treats an HCV infection.

24. The method of claim 22 or 23, wherein the HCV-infected individual is a treatment-naïve individual.

25. The method of claim 22 or 23, wherein the HCV-infected individual failed a prior treatment for HCV infection.

26. The method of any one of claims 22-25, wherein the HCV is HCV of any genotype or subtype.
